(19) United States
(12) Patent Application Publication
(10) Pub. No.: US 2016/0287592 A1
(43) Pub. Date: Oct. 6, 2016

(54) IBRUTINIB COMBINATION THERAPY

(71) Applicants: PHARMACYCLICS LLC, Sunnyvale, CA (US); JANSSEN PHARMACEUTICA NV, Beerse (BE)

(72) Inventors: Betty CHANG, Sunnyvale, CA (US); Sriram BALASUBRAMANIAN, Sunnyvale, CA (US); Richard CROWLEY, Sunnyvale, CA (US); Hsu-Ping KUO, Sunnyvale, CA (US); Brett HALL, Spring House, PA (US); Kate A. SASSER, Spring House, PA (US); Tineke CASNEUF, Beerse (BE); Michael SHAFFER, Spring House, PA (US); Matthias VERSELE, Beerse (BE); Willem LIGTENBERG, Beerse (BE); Cuc DAVIS, Spring House, PA (US)

(21) Appl. No.: 14/778,536

(22) PCT Filed: Apr. 8, 2014
(86) PCT No.: PCT/US2014/033378

§ 371 Date: Sep. 18, 2015

Related U.S. Application Data
(60) Provisional application No. 61/809,810, filed on Apr. 8, 2013.

(51) Int. Cl. A61K 31/519; A61K 31/454; A61K 31/5383; A61K 31/496; A61K 31/4439; A61K 31/5377; A61K 31/395; A61K 39/395; A61K 31/664; A61K 31/704; A61K 31/4745; A61K 31/573; A61K 31/7048; A61K 31/7068; A61K 31/282; A61K 45/06

(52) U.S. Cl. CPC A61K 31/519; A61K 45/06; A61K 31/454; A61K 31/5383; A61K 31/496; A61K 31/4439; A61K 31/5377; A61K 31/395; A61K 39/3955; A61K 31/664; A61K 31/704; A61K 31/4745; A61K 31/573; A61K 31/7048; A61K 31/7068; A61K 31/282; A61K 2039/505

(57) ABSTRACT

Combinations of Bruton's tyrosine kinase (Btk) inhibitors, e.g., 1-((R)-3-(4-amino-phenoxyphenyl)-1H-pyrazolo[3,4-d]pyrimidin-1-yl)piperidin-1-yl)prop-2-en-1-one, with a second anticancer agent are provided. Also provided are methods of treating cancers, and autoimmune disorders by administering combinations of Bruton's tyrosine kinase (Btk) inhibitors, e.g., 1-((R)-3-(4-amino-3-(4-phenoxyphenyl)-1H-pyrazolo[3,4-d]pyrimidin-1-yl)piperidin-1-yl)prop-2-en-1-one, and second anticancer agents.

### RELATED APPLICATIONS

This application claims the benefit of U.S. provisional patent application No. 61/809,810 entitled “IBRUTINIB COMBINATION THERAPY” filed on Apr. 8, 2013, which is herein incorporated by reference in its entirety.

### BACKGROUND OF THE INVENTION

Bruton's tyrosine kinase (Btk), a member of the Tec family of non-receptor tyrosine kinases, is a key signaling enzyme expressed in all hematopoietic cells types except T lymphocytes and natural killer cells. Btk plays an essential role in the B-cell signaling pathway linking cell surface B-cell receptor (BCR) stimulation to downstream intracellular responses.

Btk is a key regulator of B-cell development, activation, signaling, and survival. In addition, Btk plays a role in a number of other hematopoietic cell signaling pathways, e.g., Toll like receptor (TLR) and cytokine receptor-mediated TNF-α production in macrophages, IgE receptor signaling in Mast cells, inhibition of Fas/APO-1 apoptotic signaling in B-lineage lymphoid cells, and collagen-stimulated platelet aggregation.

1-((R)-3-(4-amino-3-(4-phenoxyphenyl)-1H-pyrazolo[3,4-d]pyrimidin-1-yl)piperidin-1-yl)prop-2-en-1-one is also known by its IUPAC name as 1-{(3R)-3-[4-amino-3-(4-phenoxyphenyl)-1H-pyrazolo[3,4-d]pyrimidin-1-yl]piperidin-1-yl}prop-2-en-1-one or 2-Propen-1-one, 1-[(3R)-3-[4-amino-3-(4-phenoxyphenyl)-1H-pyrazolo[3,4-d]pyrimidin-1-yl]-1-piperidinyl-, and has been given the USAN name, Ibrutinib. The various names given for Ibrutinib are used interchangeably herein.

### SUMMARY OF THE INVENTION

Disclosed herein, in some embodiments, is a method for treating a B-cell proliferative disorder comprising administering to a subject in need thereof a therapeutically effective amount of a combination comprising: a. Ibrutinib; and b. a second anticancer agent, wherein the anticancer agent inhibits Bcl-2; Janus kinase 2 (JAK2); Anaplastic lymphoma kinase (ALK); or heat shock protein 90 (Hsp90), wherein the combination provides a synergistic therapeutic effect compared to administration of ibrutinib or the anticancer agent alone. In some embodiments, the Ibrutinib is in a therapeutically effective amount. In some embodiments, the anticancer agent inhibits Bcl-2. In some embodiments, the anticancer agent that inhibits Bcl-2 is selected from ABT-737, ABT-199 and HA14-1. In some embodiments, the anticancer agent inhibits JAK2. In some embodiments, the anticancer agent that inhibits JAK2 is TG-101348. In some embodiments, the anticancer agent inhibits ALK. In some embodiments, the anticancer agent that inhibits ALK is NVP-TAE684. In some embodiments, the anticancer agent inhibits Hsp90. In some embodiments, the anticancer agent that inhibits Hsp 90 is 17-DMAG. In some embodiments, the B-cell proliferative disorder is diffuse large B-cell lymphoma (DLBCL), chronic lymphocytic leukemia (CLL), small lymphocytic lymphoma (SLL), high risk CLL, or a non-CLL/SLL lymphoma, follicular lymphoma, mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma, acute or chronic myelogenous (or myeloid) leukemia, myelodysplastic syndrome, or acute lymphoblastic leukemia. In some embodiments, the B-cell proliferative disorder is DLBCL. In some embodiments, the DLBCL is “activated B-cell” (ABC) DLBCL. In some embodiments, the DLBCL is “germinal center B-cell like” (GCB) DLBCL. In some embodiments, the therapeutically-effective amount of Ibrutinib is between about 10 mg to about 100 mg, 100 mg and about 200 mg, or about 200 to about 300 mg, or about 300 to about 500 mg, or about 500 to about 840 mg. In some embodiments, the therapeutically-effective amount of Ibrutinib is about 140 mg. In some embodiments, the anticancer agent is administered in an amount between about 5 mg to about 1000 mg. In some embodiments, Ibrutinib and the anticancer agent are in a combined dosage form. In some embodiments, Ibrutinib and the anticancer agent are in separate dosage forms. In some embodiments, Ibrutinib and the anticancer agent are administered concurrently. In some embodiments, Ibrutinib and the anticancer agent are administered simultaneously, essentially simultaneously or within the same treatment protocol. In some embodiments, Ibrutinib and the anticancer agent are administered sequentially. In some embodiments, the ratio of Ibrutinib to the anticancer agent is about 9:1, about 4:1, about 7:3, about 3:2, about 1:1, about 2:3, about 3:7, about 1:4, or about 1:9.

Disclosed herein, in some embodiments, is a method for treating a B-cell proliferative disorder comprising administering to a subject in need thereof a therapeutically effective amount of a combination comprising: a. Ibrutinib; and b. a second anticancer agent, wherein the anticancer agent is a glucocorticoid, a vinca alkaloid, an anti-metabolite, a DNA damaging agent, lenalidomide, rituximab, or a PKC perturbagen, wherein the combination provides a synergistic therapeutic effect compared to administration of ibrutinib or the anticancer agent alone. In some embodiments, ibrutinib is in a therapeutically effective amount. In some embodiments, the anticancer agent is a glucocorticoid. In some embodiments, the anticancer agent is selected from dexamethasone and prednisolone. In some embodiments, the anticancer agent is a vinca alkaloid. In some embodiments, the anticancer agent is vincristine. In some embodiments, the anticancer agent is an anti-metabolite. In some embodiments, the anticancer agent is gemcitabine. In some embodiments, the anticancer agent is a DNA damaging agent. In some embodiments, the DNA damaging agent is selected from carboplatin and chlorambucil. In some embodiments, the anticancer agent is lenalidomide. In some embodiments, the anticancer agent is rituximab. In some embodiments, the anticancer agent is a PKC perturbagen. In some embodiments, the PKC perturbagen is selected from enzastarin and GF109203X. In some embodiments, the B-cell proliferative disorder is diffuse large B-cell lymphoma (DLBCL), chronic lymphocytic leukemia (CLL), small lymphocytic lymphoma (SLL), high risk CLL, or a non-CLL/SLL lymphoma, follicular lymphoma, mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma, acute or chronic myelogenous (or myeloid) leukemia, myelodysplastic syndrome, or acute lymphoblastic leukemia. In some embodiments, the B-cell proliferative disorder is DLBCL. In some embodiments, the DLBCL is “activated B-cell” (ABC) DLBCL. In some embodiments, the DLBCL is “germinal center B-cell like” (GCB) DLBCL. In some embodiments, the therapeutically-effective amount of Ibrutinib is between about 10 mg to about 100 mg, 100 mg and about 200 mg, or about 200 to about 300 mg, or about 300 to about 500 mg, or about 500 to about 840 mg. In some embodiments, the therapeutically-effective amount of Ibrutinib is about 140 mg. In some embodiments, the anticancer agent is administered in an amount between about 5 mg to about 1000 mg. In some embodiments, Ibrutinib and the anticancer agent are in a combined dosage form. In some embodiments, Ibrutinib and the anticancer agent are in separate dosage forms. In some embodiments, Ibrutinib and the anticancer agent are administered concurrently. In some embodiments, Ibrutinib and the anticancer agent are administered simultaneously, essentially simultaneously or within the same treatment protocol. In some embodiments, Ibrutinib and the anticancer agent are administered sequentially. In some embodiments, the ratio of Ibrutinib to the anticancer agent is about 9:1, about 4:1, about 7:3, about 3:2, about 1:1, about 2:3, about 3:7, about 1:4, or about 1:9.

Disclosed herein, in some embodiments, is a method for treating a B-cell proliferative disorder comprising administering to a subject in need thereof a therapeutically effective amount of a combination comprising: a. Ibrutinib; and b. a second anticancer agent, wherein the anticancer agent inhibits a B-cell receptor pathway kinase selected from among Lyn/Fyn, Syk, PI3K, PKCβ, and IKK, wherein the combination provides a synergistic therapeutic effect compared to administration of ibrutinib or the anticancer agent alone. In some embodiments, ibrutinib is in a therapeutically effective amount. In some embodiments, the anticancer agent inhibits a B-cell receptor pathway kinase selected from among Lyn/Fyn, Syk, PI3K, PKCβ, and IKK. In some embodiments, the anticancer agent inhibits Lyn/Fyn. In some embodiments, the anticancer agent inhibits Syk. In some embodiments, the anticancer agent is R406. In some embodiments, the anticancer agent inhibits PKCβ. In some embodiments, the anticancer agent inhibits IKK. In some embodiments, the anticancer agent inhibits PI3K. In some embodiments, the anticancer agent that inhibits PI3K is selected from IPI-145, BKM120, BEZ235, GDC-0941, AMG319, CAL-101 and A66. In some embodiments, the B-cell proliferative disorder is diffuse large B-cell lymphoma (DLBCL), chronic lymphocytic leukemia (CLL), small lymphocytic lymphoma (SLL), high risk CLL, or a non-CLL/SLL lymphoma, follicular lymphoma, mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma, acute or chronic myelogenous (or myeloid) leukemia, myelodysplastic syndrome, or acute lymphoblastic leukemia. In some embodiments, the B-cell proliferative disorder is DLBCL. In some embodiments, the DLBCL is “activated B-cell” (ABC) DLBCL. In some embodiments, the DLBCL is “germinal center B-cell like” (GCB) DLBCL. In some embodiments, the therapeutically-effective amount of Ibrutinib is between about 10 mg to about 100 mg, 100 mg and about 200 mg, or about 200 to about 300 mg, or about 300 to about 500 mg, or about 500 to about 840 mg. In some embodiments, the therapeutically-effective amount of Ibrutinib is about 140 mg. In some embodiments, the anticancer agent is administered in an amount between about 5 mg to about 1000 mg. In some embodiments, Ibrutinib and the anticancer agent are in a combined dosage form. In some embodiments, Ibrutinib and the anticancer agent are in separate dosage forms. In some embodiments, Ibrutinib and the anticancer agent are administered concurrently. In some embodiments, Ibrutinib and the anticancer agent are administered simultaneously, essentially simultaneously or within the same treatment protocol. In some embodiments, Ibrutinib and the anticancer agent are administered sequentially. In some embodiments, the ratio of Ibrutinib to the anticancer agent is about 9:1, about 4:1, about 7:3, about 3:2, about 1:1, about 2:3, about 3:7, about 1:4, or about 1:9.

Disclosed herein, in some embodiments, is a method for treating a B-cell proliferative disorder comprising administering to a subject in need thereof a therapeutically effective amount of a combination comprising: a. Ibrutinib; and b. a second anticancer agent, wherein the anticancer agent inhibits the 20s proteasome, IRF-4, IRAK4, EZH2, CXCR4, CXCR5, GLS, cyclin dependent kinase 4/6 (CDK4/6), topoisomerase II, PLK; DNA methyltransferase, the Ras/MAPK pathway, or FGFR1 tyrosine kinase, wherein the combination provides a synergistic therapeutic effect compared to administration of ibrutinib or the anticancer agent alone. In some embodiments, ibrutinib is in a therapeutically effective amount. In some embodiments, the anticancer agent inhibits the 20s proteasome. In some embodiments, the anticancer agent is carfilzomib. In some embodiments, the anticancer agent inhibits IRF-4. In some embodiments, the anticancer agent is LEN. In some embodiments, the anticancer agent inhibits IRAK4. In some embodiments, the anticancer agent is ND-2158. In some embodiments, the anticancer agent inhibits EZH2. In some embodiments, the anticancer agent is selected from EI1, GSK343 and EPZ005687. In some embodiments, the anticancer agent inhibits CXCR4. In some embodiments, the anticancer agent is AMD3100. In some embodiments, the anticancer agent inhibits CXCR5. In some embodiments, the anticancer agent is an antibody against CXCR5. In some embodiments, wherein the anticancer agent inhibits GLS. In some embodiments, the anticancer agent is JNJ-16. In some embodiments, wherein the anticancer agent inhibits CDK4/6. In some embodiments, the anticancer agent is JNJ-08. In some embodiments, the anticancer agent inhibits topoisomerase II. In some embodiments, the anticancer agent is selected from doxorubicin and etoposide. In some embodiments, the anticancer agent inhibits PLK. In some embodiments, the anticancer agent is selected from BI-2536 and GSK461364. In some embodiments, the anticancer agent inhibits DNA methyltransferase. In some embodiments, the anticancer agent is azacitidine. In some embodiments, the anticancer agent inhibits the Ras/MAPK pathway. In some embodiments, the anticancer agent is selected from sorafenib and PLX-4032. In some embodiments, the anticancer agent inhibits FGFR1 tyrosine kinase. In some embodiments, the anticancer agent is JNJ-13. In some embodiments, the B-cell proliferative disorder is diffuse large B-cell lymphoma (DLBCL), chronic lymphocytic leukemia (CLL), small lymphocytic lymphoma (SLL), high risk CLL, or a non-CLL/SLL lymphoma, follicular lymphoma, mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma, acute or chronic myelogenous (or myeloid) leukemia, myelodysplastic syndrome, or acute lymphoblastic leukemia. In some embodiments, the B-cell proliferative disorder is DLBCL. In some embodiments, the DLBCL is “activated B-cell” (ABC) DLBCL. In some embodiments, the DLBCL is “germinal center B-cell like” (GCB) DLBCL. In some embodiments, the therapeutically-effective amount of Ibrutinib is between about 10 mg to about 100 mg, 100 mg and about 200 mg, or about 200 to about 300 mg, or about 300 to about 500 mg, or about 500 to about 840 mg. In some embodiments, the therapeutically-effective amount of Ibrutinib is about 140 mg. In some embodiments, the anticancer agent is administered in an amount between about 5 mg to about 1000 mg. In some embodiments, Ibrutinib and the anticancer agent are in a combined dosage form. In some embodiments, Ibrutinib and the anticancer agent are in separate dosage forms. In some embodiments, Ibrutinib and the anticancer agent are administered concurrently. In some embodiments, Ibrutinib and the anticancer agent are administered simultaneously, essentially simultaneously or within the same treatment protocol. In some embodiments, Ibrutinib and the anticancer agent are administered sequentially. In some embodiments, the ratio of Ibrutinib to the anticancer agent is about 9:1, about 4:1, about 7:3, about 3:2, about 1:1, about 2:3, about 3:7, about 1:4, or about 1:9.

Disclosed herein, in some embodiments, is a method for treating a B-cell proliferative disorder comprising administering to a subject in need thereof a therapeutically effective amount of a combination comprising: a. Ibrutinib; and b. a second anticancer agent, wherein the anticancer agent is selected from AZD0503, dasatinib and nilotinib, and JNJ-20, wherein the combination provides a synergistic therapeutic effect compared to administration of ibrutinib or the anticancer agent alone. In some embodiments, ibrutinib is in a therapeutically effective amount. In some embodiments, the anticancer agent is AZD0503. In some embodiments, the anticancer agent is dasatinib. In some embodiments, the anticancer agent is nilotinib. In some embodiments, the anticancer agent is JNJ-20. In some embodiments, the B-cell proliferative disorder is diffuse large B-cell lymphoma (DLBCL), chronic lymphocytic leukemia (CLL), small lymphocytic lymphoma (SLL), high risk CLL, or a non-CLL/SLL lymphoma, follicular lymphoma, mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma, acute or chronic myelogenous (or myeloid) leukemia, myelodysplastic syndrome, or acute lymphoblastic leukemia. In some embodiments, the B-cell proliferative disorder is DLBCL. In some embodiments, the DLBCL is “activated B-cell” (ABC) DLBCL. In some embodiments, the DLBCL is “germinal center B-cell like” (GCB) DLBCL. In some embodiments, the therapeutically-effective amount of Ibrutinib is between about 10 mg to about 100 mg, 100 mg and about 200 mg, or about 200 to about 300 mg, or about 300 to about 500 mg, or about 500 to about 840 mg. In some embodiments, the therapeutically-effective amount of Ibrutinib is about 140 mg. In some embodiments, the anticancer agent is administered in an amount between about 5 mg to about 1000 mg. In some embodiments, Ibrutinib and the anticancer agent are in a combined dosage form. In some embodiments, Ibrutinib and the anticancer agent are in separate dosage forms. In some embodiments, Ibrutinib and the anticancer agent are administered concurrently. In some embodiments, Ibrutinib and the anticancer agent are administered simultaneously, essentially simultaneously or within the same treatment protocol. In some embodiments, Ibrutinib and the anticancer agent are administered sequentially. In some embodiments, the ratio of Ibrutinib to the anticancer agent is about 9:1, about 4:1, about 7:3, about 3:2, about 1:1, about 2:3, about 3:7, about 1:4, or about 1:9.

Disclosed herein, in some embodiments, is a pharmaceutical composition comprising: a. a therapeutically effective amount of Ibrutinib; and b. an anticancer agent, wherein the anticancer agent inhibits Bcl-2, Janus kinase 2 (JAK2), Anaplastic lymphoma kinase (ALK), or heat shock protein 90 (Hsp90); or the anticancer agent is a glucocorticoid, a vinca alkaloid, an anti-metabolite, a DNA damaging agent, lenalidomide, rituximab, or a PKC perturbagen; or the anticancer agent inhibits a B-cell receptor pathway kinase selected from among Lyn/Fyn, Syk, PI3K, PKCβ, and IKK; or the anticancer agent inhibits the 20s proteasome, IRF-4, IRAK4, EZH2, CXCR4, CXCR5, GLS, cyclin dependent kinase 4/6 (CDK4/6), topoisomerase II, PLK; DNA methyltransferase, the Ras/MAPK pathway, or FGFR1 tyrosine kinase; or the anticancer agent is selected from AZD0503, dasatinib and nilotinib, and JNJ-20; wherein the combination provides a synergistic therapeutic effect compared to administration of ibrutinib or the anticancer agent alone. In some embodiments, the composition further comprises a pharmaceutically acceptable carrier or an adjuvant. In some embodiments, the anticancer agent inhibits Bcl-2; Janus kinase 2 (JAK2); Anaplastic lymphoma kinase (ALK); or heat shock protein 90 (Hsp90). In some embodiments, the anticancer agent is a glucocorticoid, a vinca alkaloid, an anti-metabolite, a DNA damaging agent, lenalidomide, rituximab, or a PKC perturbagen. In some embodiments, the anticancer agent inhibits a B-cell receptor pathway kinase selected from among Lyn/Fyn, Syk, PI3K, PKCβ, and IKK. In some embodiments, the anticancer agent inhibits the 20s proteasome, IRF-4, IRAK4, EZH2, CXCR4, CXCR5, GLS, cyclin dependent kinase 4/6 (CDK4/6), topoisomerase II, PLK; DNA methyltransferase, the Ras/MAPK pathway, or FGFR1 tyrosine kinase. In some embodiments, the therapeutically-effective amount of Ibrutinib is between about 10 mg to about 100 mg, 100 mg and about 200 mg, or about 200 to about 300 mg, or about 300 to about 500 mg, or about 500 to about 840 mg. In some embodiments, the therapeutically-effective amount of Ibrutinib is about 140 mg. In some embodiments, the anticancer agent is administered in an amount between about 5 mg to about 1000 mg. In some embodiments, the anticancer agent inhibits Bcl-2. In some embodiments, the anticancer agent that inhibits Bcl-2 is selected from ABT-737, ABT-199 and HA14-1. In some embodiments, the anticancer agent inhibits JAK2. In some embodiments, the anticancer agent that inhibits JAK2 is TG-101348. In some embodiments, the anticancer agent inhibits ALK. In some embodiments, the anticancer agent that inhibits ALK is NVP-TAE684. In some embodiments, the anticancer agent inhibits Hsp90. In some embodiments, the anticancer agent that inhibits Hsp 90 is 17-DMAG. In some embodiments, the anticancer agent is a glucocorticoid. In some embodiments, the anticancer agent is selected from dexamethasone and prednisolone. In some embodiments, the anticancer agent is a vinca alkaloid. In some embodiments, the anticancer agent is vincristine. In some embodiments, the anticancer agent is an anti-metabolite. In some embodiments, the anticancer agent is gemcitabine. In some embodiments, the anticancer agent is a DNA damaging agent. In some embodiments, the DNA damaging agent is selected from carboplatin and chlorambucil. In some embodiments, the anticancer agent is lenalidomide. In some embodiments, the anticancer agent is rituximab. In some embodiments, the anticancer agent is a PKC perturbagen. In some embodiments, the PKC perturbagen is selected from enzastarin and GF109203X. In some embodiments, the anticancer agent inhibits a B-cell receptor pathway kinase selected from among Lyn/Fyn, Syk, PI3K, PKCβ, and IKK. In some embodiments, the anticancer agent inhibits Lyn/Fyn. In some embodiments, the anticancer agent inhibits Syk. In some embodiments, the anticancer agent is R406. In some embodiments, the anticancer agent inhibits PKCβ. In some embodiments, the anticancer agent inhibits IKK. In some embodiments, the anticancer agent inhibits PI3K. In some embodiments, the anticancer agent that inhibits PI3K is selected from IPI-145, BKM120, BEZ235, GDC-0941, AMG319, CAL-101 and A66. In some embodiments, the anticancer agent inhibits the 20s proteasome. In some embodiments, the anticancer agent is carfilzomib. In some embodiments, the anticancer agent inhibits IRF-4. In some embodiments, the anticancer agent is LEN. In some embodiments, the anticancer agent inhibits IRAK4. In some embodiments, the anticancer agent is ND-2158. In some embodiments, the anticancer agent inhibits EZH2. In some embodiments, the anticancer agent is selected from EI1, GSK343 and EPZ005687. In some embodiments, the anticancer agent inhibits CXCR4. In some embodiments, the anticancer agent is AMD3100. In some embodiments, the anticancer agent inhibits CXCR5. In some embodiments, the anticancer agent is an antibody against CXCR5. In some embodiments, wherein the anticancer agent inhibits GLS. In some embodiments, the anticancer agent is JNJ-16. In some embodiments, wherein the anticancer agent inhibits CDK4/6. In some embodiments, the anticancer agent is JNJ-08. In some embodiments, the anticancer agent inhibits topoisomerase II. In some embodiments, the anticancer agent is selected from doxorubicin and etoposide. In some embodiments, the anticancer agent inhibits PLK. In some embodiments, the anticancer agent is selected from BI-2536 and GSK461364. In some embodiments, the anticancer agent inhibits DNA methyltransferase. In some embodiments, the anticancer agent is azacitidine. In some embodiments, the anticancer agent inhibits the Ras/MAPK pathway. In some embodiments, the anticancer agent is selected from sorafenib and PLX-4032. In some embodiments, the anticancer agent inhibits FGFR1 tyrosine kinase. In some embodiments, the anticancer agent is JNJ-13. In some embodiments, ibrutinib is in a therapeutically effective amount. In some embodiments, the anticancer agent is AZD0503. In some embodiments, the anticancer agent is dasatinib. In some embodiments, the anticancer agent is nilotinib. In some embodiments, the anticancer agent is JNJ-20.

### BRIEF DESCRIPTION OF THE FIGURES

FIG. 1 exemplifies the effect of ibrutinib alone or in combination with the IRF-4 inhibitor Lenalidomide (Len) or the IRAK4 inhibitor ND2158 on cell growth inhibition in TMD8 WT or TMD8 ibrutinib resistant cells. (A) Ibrutinib with or without Lenalidomide in TMD8 WT cells; (B) Ibrutinib with or without ND2158 in TMD8 WT cells; (C) Ibrutinib with or without Lenalidomide in TMD8 R cells; (D) Ibrutinib with or without ND2158 in TMD8 R cells.

FIG. 2 exemplifies the effect of ibrutinib alone or in combination with the IRF-4 inhibitor Lenalidomide (Len) or the IRAK4 inhibitor ND2158 on cell growth inhibition in HBL1 or Ly10 cells. (A) Ibrutinib with or without Lenalidomide in HBL1 cells; (B) Ibrutinib with or without ND2158 in HBL1 cells; (C) Ibrutinib with or without Lenalidomide in Ly10 cells; (D) Ibrutinib with or without ND2158 in Ly10 cells.

FIG. 3 exemplifies the effect of ibrutinib alone or in combination with the IRF-4 inhibitor Lenalidomide (Len) or the IRAK4 inhibitor ND2158 on cell growth inhibition in Ly3 or DHL2 cells. (A) Ibrutinib with or without Lenalidomide in Ly3 cells; (B) Ibrutinib with or without ND2158 in Ly3 cells; (C) Ibrutinib with or without Lenalidomide in DHL2 cells; (D) Ibrutinib with or without ND2158 in DHL2 cells.

FIG. 4 exemplifies the effect of ibrutinib alone or in combination with the IRF-4 inhibitor Lenalidomide (Len) or the IRAK4 inhibitor ND2158 on cell growth inhibition in U2932 cells. (A) Ibrutinib with or without Lenalidomide in U2932 cells; (B) Ibrutinib with or without ND2158 in Ly3 cells.

FIG. 5 exemplifies the effect of ibrutinib alone or in combination with the SYK inhibitor R406 on cell growth inhibition in TMD8 WT, TMD8 ibrutinib resistant, HBL1 or Ly10 cells. (A) Ibrutinib with or without R406 in TMD8 WT cells; (B) Ibrutinib with or without R406 in TMD8-R cells; (C) Ibrutinib with or without R406 in HBL1 cells; (D) Ibrutinib with or without R406 in Ly10 cells.

FIG. 6 exemplifies the effect of ibrutinib alone or in combination with the SYK inhibitor R406 on cell growth inhibition in Ly3, DHL2, or U2932 cells. (A) Ibrutinib with or without R406 in Ly3 cells; (B) Ibrutinib with or without R406 in DHL2 cells; (C) Ibrutinib with or without R406 in HBL1 cells; (D) Ibrutinib with or without R406 in U2932 cells.

FIG. 7 exemplifies the effect of ibrutinib alone or in combination with the BCL-2 inhibitor ABT-199 on cell growth inhibition in TMD8 WT or TMD8 ibrutinib resistant cells. (A) Ibrutinib with or without ABT-199 in TMD8 WT cells; (B) Ibrutinib with or without ABT-199 in TMD8-R cells.

FIG. 8 exemplifies the effect of ibrutinib (ib) alone or in combination with the BCL-2 inhibitor ABT-199 on cell growth inhibition in TMD8 WT, TMD8 ibrutinib resistant, or HBL1 cells. (A) Ibrutinib with or without ABT-199 in TMD8 WT cells; (B) Ibrutinib with or without ABT-199 in TMD8-R cells; (C) Ibrutinib with or without ABT-199 in HBL1 cells.

FIG. 9 exemplifies the effect of ibrutinib (ib) alone or in combination with the BCL-2 inhibitor ABT-199 on cell growth inhibition in Ly3, Ly10, DHL2, or U2932 cells. (A) Ibrutinib with or without ABT-199 in Ly3 cells; (B) Ibrutinib with or without ABT-199 in Ly10 cells; (C) Ibrutinib with or without ABT-199 in DHL2 cells; (D) Ibrutinib with or without ABT-199 in U2932 cells.

FIG. 10 exemplifies the effect of ibrutinib alone or in combination with EZH2 inhibitors EI1, GSK343, or EPZ005687 on cell growth inhibition in TMD8 WT or TMD8 ibrutinib resistant cells. (A) Ibrutinib with or without EI1, GSK343, or EPZ005687 in TMD8 WT cells; (B) Ibrutinib with or without EI1, GSK343, or EPZ005687 in TMD8-R cells.

FIG. 11 exemplifies the effect of ibrutinib alone or in combination with EZH2 inhibitors EI1, GSK343, or EPZ005687 on cell growth inhibition in DHL4, DHL5, HBL1, Ly3, or Ly10 cells. (A) Ibrutinib with or without EI1, GSK343, or EPZ005687 in DHL4 cells; (B) Ibrutinib with or without EI1, GSK343, or EPZ005687 in DHL5 cells; (C) Ibrutinib with or without EI1, GSK343, or EPZ005687 in HBL1 cells; (D) Ibrutinib with or without EI1, GSK343, or EPZ005687 in Ly3 cells; (E) Ibrutinib with or without EI1, GSK343, or EPZ005687 in Ly10 cells.

FIG. 12 exemplifies the effect of ibrutinib alone or in combination with the CXCR4 inhibitor AMD3100 on cell growth inhibition in TMD8 WT or TMD8 ibrutinib resistant cells (TMD8-ibR). (A) Ibrutinib with or without AMD3100 in TMD8 WT cells; (B) Ibrutinib with or without AMD3100 in TMD8-ibR cells.

FIG. 13 exemplifies the effect of ibrutinib alone or in combination with the CXCR4 inhibitor AMD3100 on cell growth inhibition in Ly10, HBL1, Ly3, SUDHL2, or U2932 cells. (A) Ibrutinib with or without AMD3100 in Ly10 cells; (B) Ibrutinib with or without AMD3100 in HBL1 cells; (C) Ibrutinib with or without AMD3100 in Ly3 cells; (D) Ibrutinib with or without AMD3100 in SUDHL2 cells; (E) Ibrutinib with or without AMD3100 in U2932 cells.

FIG. 14 exemplifies the effect of ibrutinib in combination with an IgG antibody (control) or antibodies to PD-1 (J110, J-116, or EH12.1) on cell growth inhibition in DB, RCK8, Ly3, DHL2, U2932, TMD8 ibrutinib resistant, DHL4, DHL5, HBL1, or TMD8 cells. (A) Ibrutinib with IgG, J110, J116, or EH12.1 in DB cells; (B) Ibrutinib with IgG, J110, J116, or EH12.1 in RCK8 cells; (C) Ibrutinib with IgG, J110, J116, or EH12.1 in Ly3 cells; (D) Ibrutinib with IgG, J110, J116, or EH12.1 in DHL2 cells; (E) Ibrutinib with IgG, J110, J116, or EH12.1 in U2932 cells; (F) Ibrutinib with IgG, J110, J116, or EH12.1 in TMD8-R cells; (G) Ibrutinib with IgG, J110, J116, or EH12.1 in DHL4 cells; (H) Ibrutinib with IgG, J110, J116, or EH12.1 in DHL5 cells; (I) Ibrutinib with IgG, J110, J116, or EH12.1 in HBL1 cells; (J) Ibrutinib with IgG, J110, J116, or EH12.1 in TMD8 WT cells.

FIG. 15 exemplifies the effect of ibrutinib (Ib) in combination with an IgG antibody (control) or antibodies to PD-L1 or PD-L2 on cell growth inhibition in DB, RCK8, Ly3, DHL2, U2932, TMD8 ibrutinib resistant, DHL4, DHL5, HBL1, or TMD8 cells. (A) Ibrutinib with IgG, anti-PD-L1 or anti-PD-L2 in DB cells; (B) Ibrutinib with IgG, anti-PD-L1 or anti-PD-L2 in RCK8 cells; (C) Ibrutinib with IgG, anti-PD-L1 or anti-PD-L2 in Ly3 cells; (D) Ibrutinib with IgG, anti-PD-L1 or anti-PD-L2 in DHL2 cells; (E) Ibrutinib with IgG, anti-PD-L1 or anti-PD-L2 in U2932 cells; (F) Ibrutinib with IgG, anti-PD-L1 or anti-PD-L2 in TMD8-R cells; (G) Ibrutinib with IgG, anti-PD-L1 or anti-PD-L2 in DHL4 cells; (H) Ibrutinib with IgG, anti-PD-L1 or anti-PD-L2 in DHL5 cells; (I) Ibrutinib with IgG, anti-PD-L1 or anti-PD-L2 in HBL1 cells; (J) Ibrutinib with IgG, anti-PD-L1 or anti-PD-L2 in TMD8 WT cells.

FIG. 16 exemplifies the effect of ibrutinib (Ib) in combination with an IgG antibody (control) or an antibody to CXCR5 on cell growth inhibition in DB, RCK8, Ly3, DHL2, U2932, TMD8 ibrutinib resistant, DHL4, DHL5, HBL1, or TMD8 cells. (A) Ibrutinib with IgG or anti-CXCR5 in DB cells; (B) Ibrutinib with IgG or anti-CXCR5 in RCK8 cells; (C) Ibrutinib with IgG or anti-CXCR5 in Ly3 cells; (D) Ibrutinib with IgG or anti-CXCR5 in DHL2 cells; (E) Ibrutinib with IgG or anti-CXCR5 in U2932 cells; (F) Ibrutinib with IgG or anti-CXCR5 in TMD8-R cells; (G) Ibrutinib with IgG or anti-CXCR5 in DHL4 cells; (H) Ibrutinib with IgG or anti-CXCR5 in DHL5 cells; (I) Ibrutinib with IgG or anti-CXCR5 in HBL1 cells; (J) Ibrutinib with IgG or anti-CXCR5 in TMD8 WT cells.

FIG. 17 exemplifies the effect of ibrutinib in combination with carfilzomib on cell growth inhibition in TMD8 ibrutinib-sensitive and TMD8 ibrutinib-resistant ABC-DLBCL cells.

FIG. 18 exemplifies the synergy of twenty-one anti-cancer agents in combination with ibrutinib. JNJ-02 is ibrutinib. JNJ-03 is PCI-45292. JNJ-05 is abexinostat. Seventeen Diffuse Large B cell lymphoma (DLBCL) cell lines were tested.

FIG. 19 exemplifies the synergy of JNJ-02 in combination with glucocorticoids. FIG. 19A illustrates the synergy score heat map. Dexamethasone and prednisolone were tested in DOHH-2 (FIG. 19B), HBL-2 (FIG. 19C) and TMD8 (FIG. 19D) cell lines. JNJ-02 is ibrutinib. Dexamethasone and prednisolone demonstrate strong synergy and good breadth of activity.

FIG. 20 exemplifies the synergy of JNJ-02 in combination with vinca alkaloids. FIG. 20A illustrates the synergy score heat map. Vincristine sulfate was tested in HBL-1 (FIG. 20B), SU-DHL-8 (FIG. 20C) and OCI-Ly3 (FIG. 20D) cell lines. JNJ-02 is ibrutinib.

FIG. 21 exemplifies the synergy of JNJ-02 in combination with TOPO II inhibitors. FIG. 21A illustrates the synergy score heat map of JNJ-02 in combination with either doxorubicin HCl or etoposide. Doxorubicin HCl was tested in HBL-1 (FIG. 21B), Pfeiffer (FIG. 21C) and TMD8 (FIG. 21D) cell lines. JNJ-02 is ibrutinib.

FIG. 22 exemplifies the synergy of JNJ-02 in combination with anti-metabolite. FIG. 22A illustrates the synergy score heat map. Gemcitabine was tested in HBL-1 (FIG. 22B), OCI-Ly7 (FIG. 22C) and SU-DHL-5 (FIG. 22D) cell lines. JNJ-02 is ibrutinib.

FIG. 23 exemplifies the synergy of JNJ-02 in combination with DNA alkylating/damaging agents. FIG. 23A illustrates the synergy score heat map of JNJ-02 in combination with either chlorambucil or carboplatin. Chlorambucil was tested in TMD8 (FIG. 23B) and HBL-1 (FIG. 23C) cell lines. JNJ-02 is ibrutinib.

FIG. 24 exemplifies the synergy of JNJ-02 in combination with lenalidomide. FIG. 24A illustrates the synergy score heat map. Lenalidomide was tested in DOHH-2 (FIG. 24B-FIG. 24C), OCI-Ly18 (FIG. 24D-FIG. 24E) and TMD8 (FIG. 24F-FIG. 24G) cell lines. Lenalidomide is active as a single agent but does not show synergy with JNJ-02 in DOHH-2 and OCI-Ly18 cell lines. However, lenalidomide is not active as a single agent but synergizes with JNJ-02 in TMD8 cell line. JNJ-02 is ibrutinib.

FIG. 25 exemplifies the synergy of JNJ-02 in combination with rituximab. FIG. 25A illustrates the synergy score heat map of JNJ-02 in combination with rituximab and JNJ-0001 (siltuximab). Rituximab was tested in OCI-Ly1 (FIG. 25B), SU-DHL-6 (FIG. 25C) and DOHH-2 (FIG. 25D) cell lines. Synergy is observed with rituximab but not with JNJ-0001 (siltuximab). JNJ-02 is ibrutinib.

FIG. 26 exemplifies the synergy of JNJ-02 in combination with SYK inhibitor. FIG. 26A illustrates the synergy score heat map. R406 was tested in HBL-1 (FIG. 26B-FIG. 26C), SU-DHL-6 (FIG. 26D-FIG. 26E) and TMD8 (FIG. 26F-FIG. 26G) cell lines. JNJ-02 is ibrutinib.

FIG. 27 exemplifies the synergy of JNJ-02 in combination with PI3K pathway inhibitors. FIG. 27A illustrates the synergy score heat map. CAL-101 and A66 were tested in HT (FIG. 27B), SU-DHL-6 (FIG. 27C) and TMD8 (FIG. 27D) cell lines. JNJ-02 is ibrutinib.

FIG. 28 exemplifies the synergy of JNJ-02 in combination with NF-KB pathway inhibitors. FIG. 28A illustrates the synergy score heat map. IKK inhibitor VII and JNJ-20 were tested in TMD8 (FIG. 28B), OCI-Ly1 (FIG. 28C) and SU-DHL-8 (FIG. 28D) cell lines. IKK inhibitor VII shows strong synergy and good breadth of activity. JNJ-20 synergies in SU-DHL-8 cell line. JNJ-02 is ibrutinib.

FIG. 29 exemplifies the synergy of JNJ-02 in combination with PKC perturbagens. FIG. 29A illustrates the synergy score heat map. Enzastaurin and GF 109203X were tested in OCI-Ly18 (FIG. 29B), SU-DHL-6 (FIG. 29C) and TMD8 (FIG. 29D) cell lines. JNJ-02 is ibrutinib.

FIG. 30 exemplifies the synergy of JNJ-02 in combination with JAK inhibitor. FIG. 30A illustrates the synergy score heat map. TG-101348 was tested in HBL-1 (FIG. 30B-FIG. 30C), OCI-Ly1 (FIG. 30D-FIG. 30E) and TMD8 (FIG. 30E-FIG. 30G) cell lines. JNJ-02 is ibrutinib.

FIG. 31 exemplifies the synergy of JNJ-02 in combination with cyclin-dependent kinase 4 and 6 (Cdk4/6) inhibitor JNJ-08. FIG. 31A illustrates the synergy score heat map. JNJ-08 (Cdk4/6 inhibitor) was tested in HBL-1 (FIG. 31B-FIG. 31C), SU-DHL-6 (FIG. 31D-FIG. 31E) and TMD8 (FIG. 31F-FIG. 31G) cell lines. JNJ-02 is ibrutinib.

FIG. 32 exemplifies the synergy of JNJ-02 in combination with BCL2 inhibitors. FIG. 32A illustrates the synergy score heat map. ABT-737 and HA14-1 were tested in HBL-1 (FIG. 32B), OCI-Ly10 (FIG. 32C) and TMD8 (FIG. 32D) cell lines. ABT-737 shows strong synergy and good breadth of activity. HA14-1 shows modest synergy in selected cell lines. JNJ-02 is ibrutinib.

FIG. 33 exemplifies the synergy of JNJ-02 in combination with PLK1 inhibitors. FIG. 33A illustrates the synergy score heat map. BI 2536 and GSK461364 were tested in DOHH-2 (FIG. 33B), HBL-1 (FIG. 33C) and TMD8 (FIG. 33D) cell lines. JNJ-02 is ibrutinib.

FIG. 34 exemplifies the synergy of JNJ-02 in combination with GLS inhibitor JNJ-16 and atrovastatin. FIG. 34A illustrates the synergy score heat map. GLS inhibitor JNJ-16 and atrovastatin were tested in OCI-Ly1 (FIG. 34B), SU-DHL-6 (FIG. 34C) and TMD8 (FIG. 34D) cell lines. GLS inhibitor JNJ-16 shows strong synergy and good breadth of activity. Atrovastatin synergizes with JNJ-02. JNJ-02 is ibrutinib.

FIG. 35 exemplifies the synergy of JNJ-02 in combination with DNA methyltransferase. FIG. 35A illustrates the synergy score heat map. Azacitidine was tested in TMD8 (FIG. 35B-FIG. 35C), HBL-1 (FIG. 35D-FIG. 35E) and OCI-Ly18 (FIG. 35F-FIG. 35G) cell lines. JNJ-02 is ibrutinib.

FIG. 36 exemplifies the synergy of JNJ-02 in combination with Ras/MAPK pathway inhibitors. FIG. 36A illustrates the synergy score heat map. Sorafenib and PLX-4032 were tested in OCI-Ly1 (FIG. 36B), SU-DHL-8 (FIG. 36C) and SU-DHL-6 (FIG. 36D) cell lines. JNJ-02 is ibrutinib.

FIG. 37 exemplifies the synergy of JNJ-02 in combination with AKT/mTOR pathway inhibitors. FIG. 37A illustrates the synergy score heat map. JNJ-18 and sirolimus were tested in TMD8 (FIG. 37B), SU-DHL-6 (FIG. 37C) and OCI-Ly10 (FIG. 37D) cell lines. JNJ-02 is ibrutinib.

FIG. 38 exemplifies the synergy of JNJ-02 in combination with tyrosine kinase receptor inhibitors. FIG. 38A illustrates the synergy score heat map. AZD0530, Dasatinib, and Nilotinib were tested in TMD8 (FIG. 38B) and OCI-Ly1 (FIG. 38C) cell lines. JNJ-02 is ibrutinib.

FIG. 39 exemplifies the synergy of JNJ-02 in combination with FGFR1 tyrosine kinase inhibitor JNJ-13. FIG. 39A illustrates the synergy score heat map. JNJ-13 was tested in TMD8 (FIG. 39B-FIG. 39C), DOHH-2 (FIG. 39D-FIG. 39E) and OCI-Ly1 (FIG. 39F-FIG. 39G) cell lines. JNJ-02 is ibrutinib.

### DETAILED DESCRIPTION OF THE INVENTION

Small molecule Btk inhibitors, such as Ibrutinib, are useful for reducing the risk of or treating a variety of diseases affected by or affecting many cell types of the hematopoietic lineage including, e.g., autoimmune diseases, heteroimmune conditions or diseases, inflammatory diseases, cancer (e.g., B-cell proliferative disorders), and thromboembolic disorders.

### CERTAIN TERMINOLOGY

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

“Bioavailability” refers to the percentage of Ibrutinib dosed that is delivered into the general circulation of the animal or human being studied. The total exposure (AUC(0-∞)) of a drug when administered intravenously is usually defined as 100% bioavailable (F %). “Oral bioavailability” refers to the extent to which Ibrutinib is absorbed into the general circulation when the pharmaceutical composition is taken orally as compared to intravenous injection.

“Blood plasma concentration” refers to the concentration of Ibrutinib in the plasma component of blood of a subject. It is understood that the plasma concentration of Ibrutinib may vary significantly between subjects, due to variability with respect to metabolism and/or possible interactions with other therapeutic agents. In accordance with one embodiment disclosed herein, the blood or plasma concentration of Ibrutinib may vary from subject to subject. Likewise, values such as maximum plasma concentration (Cmax) or time to reach maximum plasma concentration (Tmax), or total area under the plasma concentration time curve (AUC(0-∞)) may vary from subject to subject. Due to this variability, the amount necessary to constitute “a therapeutically effective amount” of Ibrutinib may vary from subject to subject.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of Ibrutinib, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

The terms “subject”, “patient” and “individual” are used interchangeably. As used herein, they refer to an animal. By way of example only, a subject may be, but is not limited to, a mammal including, but not limited to, a human. The terms do not require the supervision (whether continuous or intermittent) of a medical professional.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, the IC₅₀ refers to an amount, concentration or dosage of a particular test compound that achieves a 50% inhibition of a maximal response, such as inhibition of Btk, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

### Btk Inhibitor Compounds Including Ibrutinib, and Pharmaceutically Acceptable Salts Thereof

In some embodiments, the Btk inhibitor compounds described herein are selective for Btk and kinases having a cysteine residue in an amino acid sequence position of the tyrosine kinase that is homologous to the amino acid sequence position of cysteine 481 in Btk. The Btk inhibitor compounds can form a covalent bond with Cys 481 of Btk (e.g., via a Michael reaction).

In some embodiments, the Btk inhibitor is (R)-1-(3-(4-amino-3-(4-phenoxyphenyl)-1H-pyrazolo[3,4-d]pyrimidin-1-yl)piperidin-1-yl)prop-2-en-1-one (i.e. PCI-32765/ibrutinib)

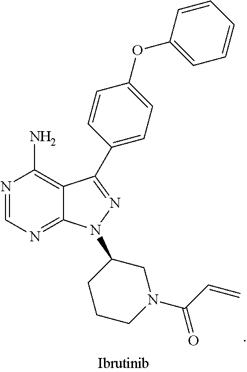 [SMILES: C=CC(=O)N1CCC[C@@H](n2nc(-c3ccc(Oc4ccccc4)cc3)c3c(N)ncnc32)C1]

In some embodiments, the Btk inhibitor is AVL-263 (Avila Therapeutics/Celgene Corporation), AVL-292 (Avila Therapeutics/Celgene Corporation), AVL-291 (Avila Therapeutics/Celgene Corporation), BMS-488516 (Bristol-Myers Squibb), BMS-509744 (Bristol-Myers Squibb), CGI-1746 (CGI Pharma/Gilead Sciences), CTA-056, GDC-0834 (Genentech), GDC-0853 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), or HM71224 (Hanmi Pharmaceutical Company Limited).

In some embodiments, the Btk inhibitor is 4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide (CGI-1746); 7-benzyl-1-(3-(piperidin-1-yl)propyl)-2-(4-(pyridin-4-yl)phenyl)-1H-imidazo[4,5-g]quinoxalin-6(5H)-one (CTA-056); (R)—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide (GDC-0834); 6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one (RN-486); N-[5-[5-(4-acetylpiperazine-1-carbonyl)-4-methoxy-2-methylphenyl]sulfanyl-1,3-thiazol-2-yl]-4-[(3,3-dimethylbutan-2-ylamino)methyl]benzamide (BMS-509744, HY-11092); or N-(5-((5-(4-Acetylpiperazine-1-carbonyl)-4-methoxy-2-methylphenyl)thio)thiazol-2-yl)-4-(((3-methylbutan-2-yl)amino)methyl)benzamide (HY11066).

In some embodiments, the Btk inhibitor is:

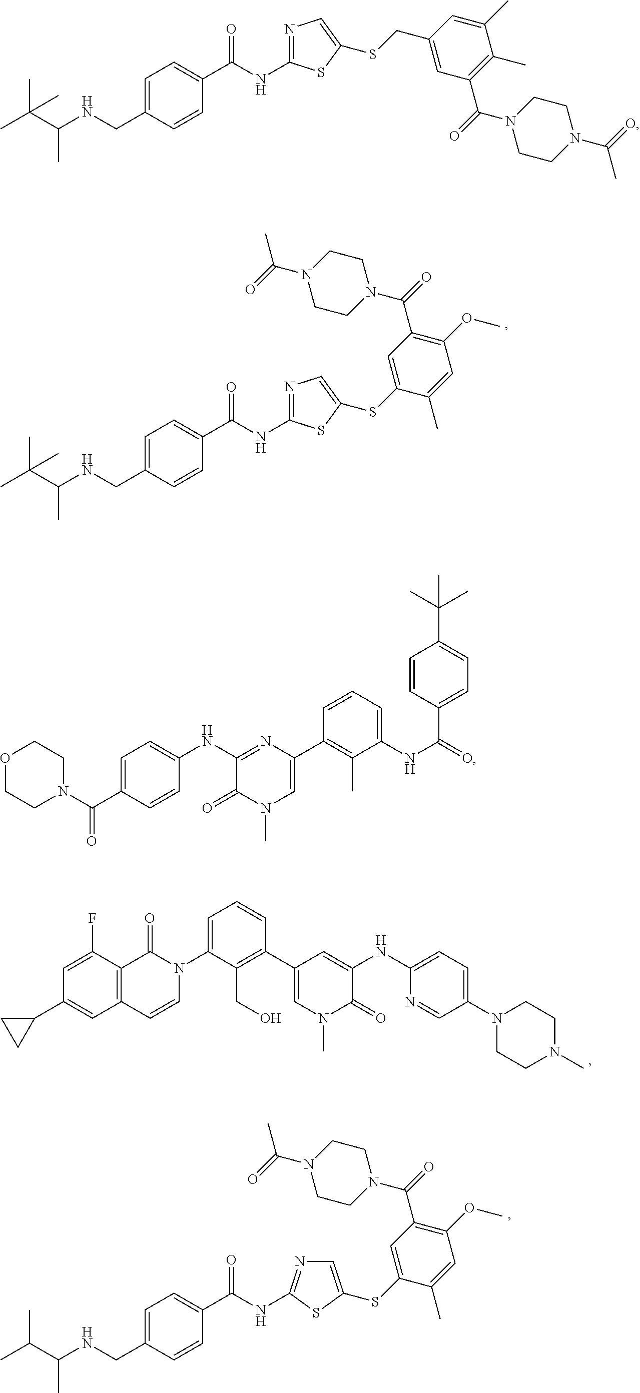

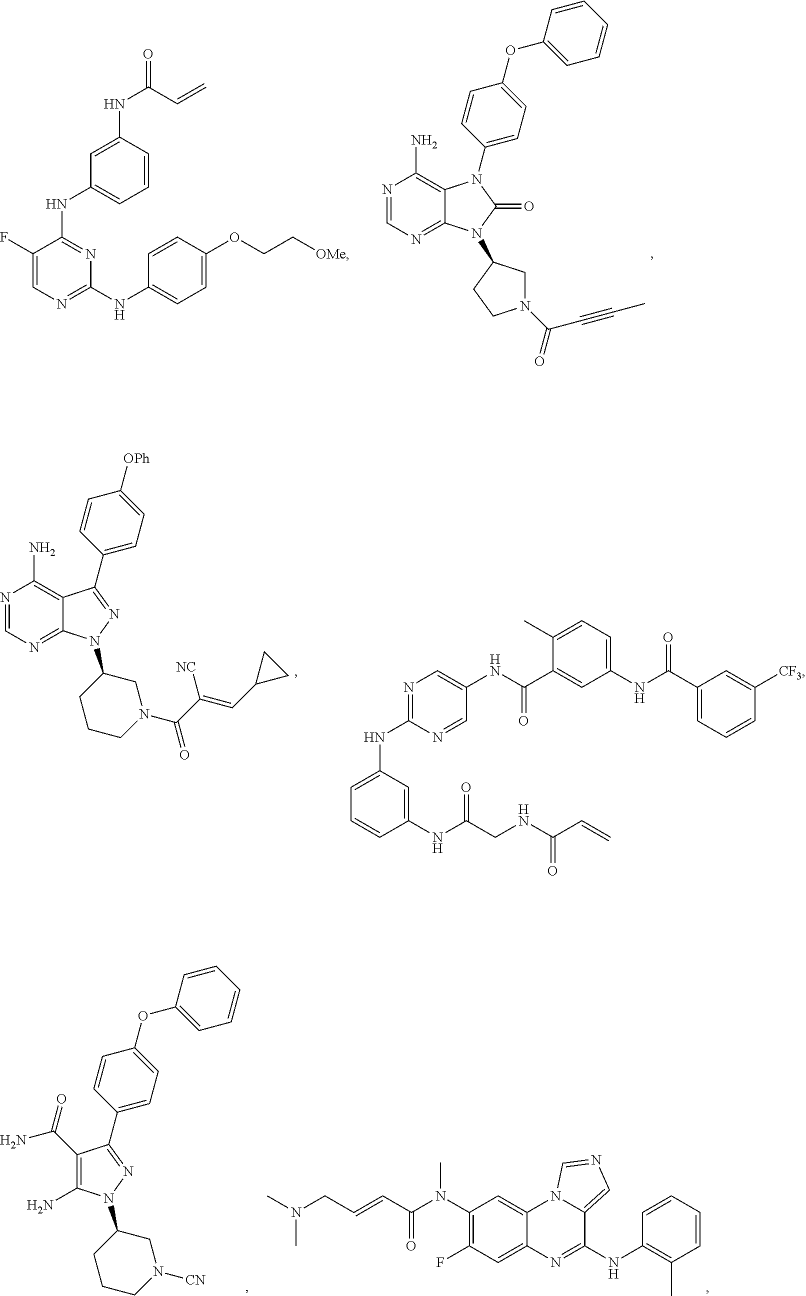 [SMILES: C=CC(=O)Nc1cccc(Nc2nc(Nc3ccc(OCCOC)cc3)ncc2F)c1 ; CC#CC(=O)N1CC[C@@H](n2c(=O)n(-c3ccc(Oc4ccccc4)cc3)c3c(N)ncnc32)C1 ; N#C/C(=C\C1CC1)C(=O)N1CCC[C@@H](n2nc(-c3ccc(Oc4ccccc4)cc3)c3c(N)ncnc32)C1 ; C=CC(=O)NCC(=O)Nc1cccc(Nc2ncc(NC(=O)c3cc(NC(=O)c4cccc(C(F)(F)F)c4)ccc3C)cn2)c1 ; N#CN1CCC[C@@H](n2nc(-c3ccc(Oc4ccccc4)cc3)c(C(N)=O)c2N)C1 ; Cc1ccccc1Nc1nc2cc(F)c(N(C)C(=O)/C=C/CN(C)C)cc2n2cncc12]

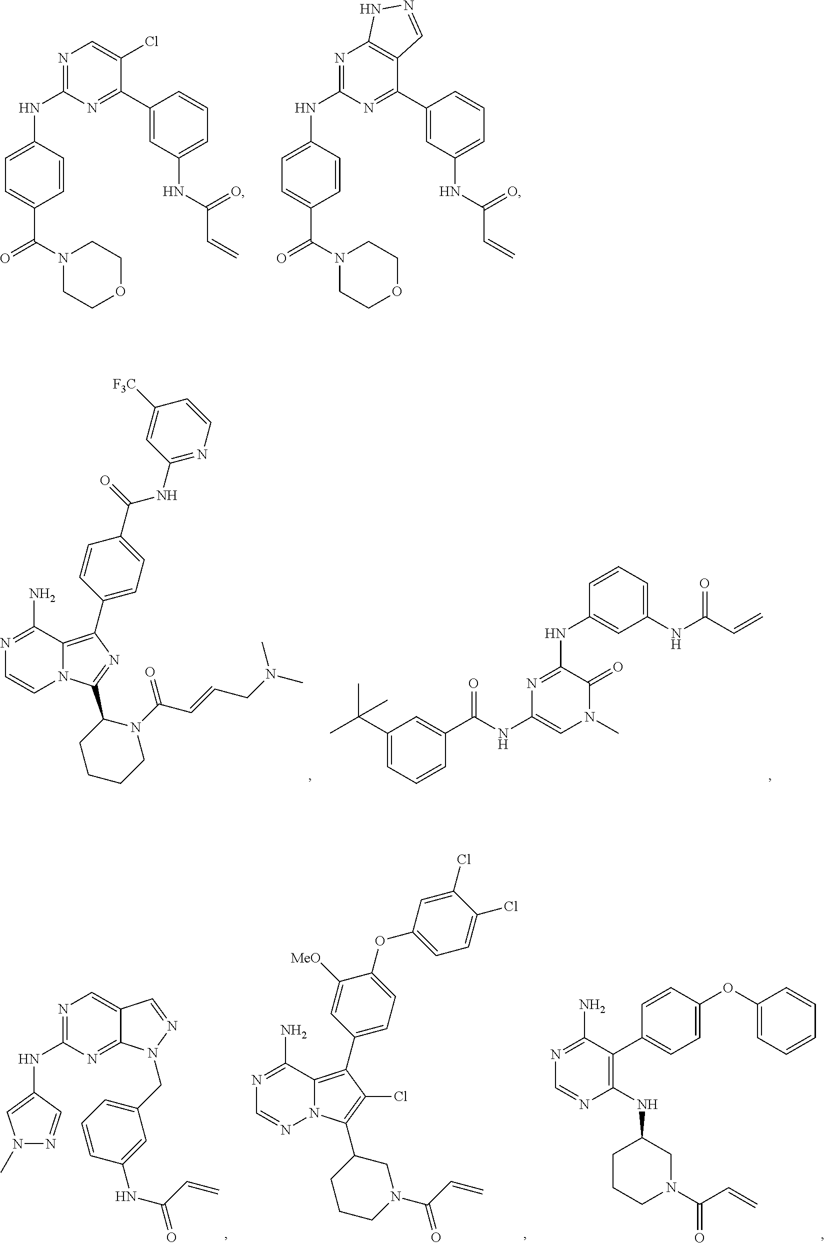

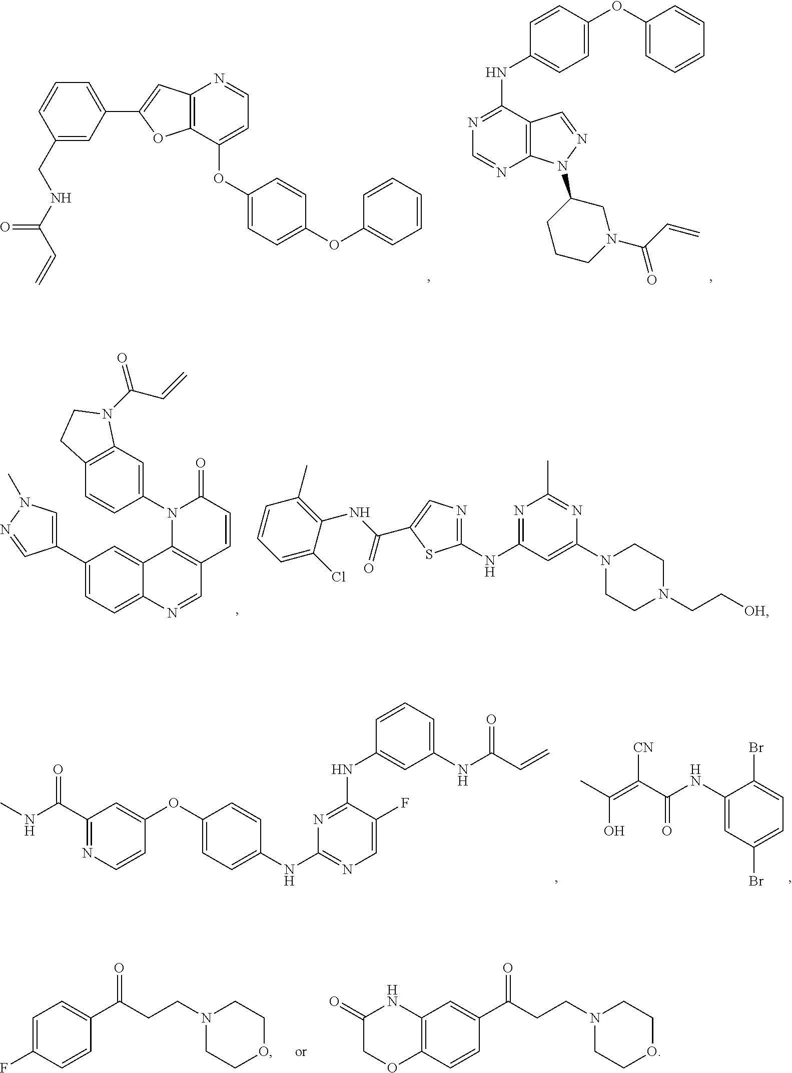

In some embodiments, the Btk inhibitor is Ibrutinib. “Ibrutinib” or “1-((R)-3-(4-amino-3-(4-phenoxyphenyl)-1H-pyrazolo[3,4-d]pyrimidin-1-yl)piperidin-1-yl)prop-2-en-1-one” or “1-{(3R)-3-[4-amino-3-(4-phenoxyphenyl)-1H-pyrazolo[3,4-d]pyrimidin-1-yl]piperidin-1-yl}prop-2-en-1-one” or “2-Propen-1-one, 1-[(3R)-3-[4-amino-3-(4-phenoxyphenyl)-1H-pyrazolo[3,4-d]pyrimidin-1-yl]-1-piperidinyl-” or Ibrutinib or any other suitable name refers to the compound with the following structure:

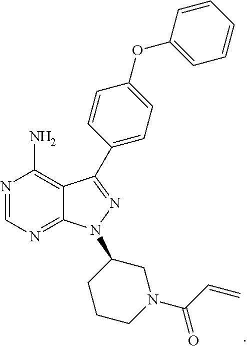 [SMILES: C=CC(=O)N1CCC[C@@H](n2nc(-c3ccc(Oc4ccccc4)cc3)c3c(N)ncnc32)C1]

PCI-45227, a metabolite of Ibrutinib, refers to 1-((R)-3-(4-amino-3-(4-phenoxyphenyl)-1H-pyrazolo[3,4-d]pyrimidin-1-yl)piperidin-1-yl)-2,3-dihydroxypropan-1-one.

A wide variety of pharmaceutically acceptable salts is formed from Ibrutinib and includes:

acid addition salts formed by reacting Ibrutinib with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like; —acid addition salts formed by reacting Ibrutinib with an inorganic acid, which includes hydrochloric acid, hydrobromic acid, sulfuric acid, nitric acid, phosphoric acid, hydroiodic acid, hydrofluoric acid, phosphorous acid, and the like.

The term “pharmaceutically acceptable salts” in reference to Ibrutinib refers to a salt of Ibrutinib, which does not cause significant irritation to a mammal to which it is administered and does not substantially abrogate the biological activity and properties of the compound.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol. In some embodiments, solvates of Ibrutinib, or pharmaceutically acceptable salts thereof, are conveniently prepared or formed during the processes described herein. In some embodiments, solvates of Ibrutinib are anhydrous. In some embodiments, Ibrutinib, or pharmaceutically acceptable salts thereof, exist in unsolvated form. In some embodiments, Ibrutinib, or pharmaceutically acceptable salts thereof, exist in unsolvated form and are anhydrous.

In yet other embodiments, Ibrutinib, or a pharmaceutically acceptable salt thereof, is prepared in various forms, including but not limited to, amorphous phase, crystalline forms, milled forms and nano-particulate forms. In some embodiments, Ibrutinib, or a pharmaceutically acceptable salt thereof, is amorphous. In some embodiments, Ibrutinib, or a pharmaceutically acceptable salt thereof, is amorphous and anhydrous. In some embodiments, Ibrutinib, or a pharmaceutically acceptable salt thereof, is crystalline. In some embodiments, Ibrutinib, or a pharmaceutically acceptable salt thereof, is crystalline and anhydrous.

In some embodiments, Ibrutinib is prepared as outlined in U.S. Pat. No. 7,514,444.

Combination with Second Anticancer Agent

Disclosed herein, in certain embodiments, are pharmaceutical combinations comprising a Btk inhibitor compound and a second anticancer agent, wherein the combination provides a synergistic therapeutic effect compared to administration of ibrutinib or the second anticancer agent alone.

In some embodiments, the second anticancer agent inhibits Bcl-2; Janus kinase 2 (JAK2); Anaplastic lymphoma kinase (ALK); or heat shock protein 90 (Hsp90), wherein the combination provides a synergistic therapeutic effect compared to administration of ibrutinib or the second anticancer agent alone. In some embodiments, the second anticancer agent inhibits Bcl-2. In some embodiments, the second anticancer agent that inhibits Bcl-2 is selected from ABT-737, ABT-199 and HA14-1. In some embodiments, the second anticancer agent inhibits JAK2. In some embodiments, the second anticancer agent that inhibits JAK2 is TG-101348. In some embodiments, the second anticancer agent inhibits ALK. In some embodiments, the second anticancer agent that inhibits ALK is NVP-TAE684. In some embodiments, the second anticancer agent inhibits Hsp90. In some embodiments, the second anticancer agent that inhibits Hsp 90 is 17-DMAG.

In some embodiments, the second anticancer agent is a glucocorticoid, a vinca alkaloid, an anti-metabolite, a DNA damaging agent, lenalidomide, rituximab, or a PKC perturbagen, wherein the combination provides a synergistic therapeutic effect compared to administration of ibrutinib or the second anticancer agent alone. In some embodiments, the second anticancer agent is a glucocorticoid. In some embodiments, the second anticancer agent is selected from dexamethasone and prednisolone. In some embodiments, the second anticancer agent is a vinca alkaloid. In some embodiments, the second anticancer agent is vincristine. In some embodiments, the second anticancer agent is an anti-metabolite. In some embodiments, the second anticancer agent is gemcitabine. In some embodiments, the second anticancer agent is a DNA damaging agent. In some embodiments, the DNA damaging agent is selected from carboplatin and chlorambucil. In some embodiments, the second anticancer agent is lenalidomide. In some embodiments, the second anticancer agent is rituximab. In some embodiments, the second anticancer agent is a PKC perturbagen. In some embodiments, the PKC perturbagen is selected from enzastarin and GF109203X.

In some embodiments, the second anticancer agent inhibits a B-cell receptor pathway kinase selected from among Lyn/Fyn, Syk, PI3K, PKCβ, and IKK, wherein the combination provides a synergistic therapeutic effect compared to administration of ibrutinib or the second anticancer agent alone. In some embodiments, the second anticancer agent inhibits a B-cell receptor pathway kinase selected from among Lyn/Fyn, Syk, PI3K, PKCβ, and IKK. In some embodiments, the second anticancer agent inhibits Lyn/Fyn. In some embodiments, the second anticancer agent inhibits Syk. In some embodiments, the second anticancer agent is R406. In some embodiments, the second anticancer agent inhibits PKCβ. In some embodiments, the second anticancer agent inhibits IKK. In some embodiments, the second anticancer agent inhibits PI3K. In some embodiments, the second anticancer agent that inhibits PI3K is selected from IPI-145, BKM120, BEZ235, GDC-0941, AMG319, CAL-101 and A66.

In some embodiments, the second anticancer agent inhibits the 20s proteasome, IRF-4, IRAK4, EZH2, CXCR4, CXCR5, GLS, cyclin dependent kinase 4/6 (CDK4/6), topoisomerase II, PLK; DNA methyltransferase, the Ras/MAPK pathway, or FGFR1 tyrosine kinase, wherein the combination provides a synergistic therapeutic effect compared to administration of ibrutinib or the second anticancer agent alone. In some embodiments, the second anticancer agent inhibits the 20s proteasome. In some embodiments, the second anticancer agent is carfilzomib. In some embodiments, the second anticancer agent inhibits IRF-4. In some embodiments, the second anticancer agent is LEN. In some embodiments, the second anticancer agent inhibits IRAK4. In some embodiments, the second anticancer agent is ND-2158. In some embodiments, the second anticancer agent inhibits EZH2. In some embodiments, the second anticancer agent is selected from EI1, GSK343 and EPZ005687. In some embodiments, the second anticancer agent inhibits CXCR4. In some embodiments, the second anticancer agent is AMD3100. In some embodiments, the second anticancer agent inhibits CXCR5. In some embodiments, the second anticancer agent is an antibody against CXCR5. In some embodiments, wherein the second anticancer agent inhibits GLS. In some embodiments, the second anticancer agent is JNJ-16. In some embodiments, wherein the second anticancer agent inhibits CDK4/6. In some embodiments, the second anticancer agent is JNJ-08. In some embodiments, the second anticancer agent inhibits topoisomerase II. In some embodiments, the second anticancer agent is selected from doxorubicin and etoposide. In some embodiments, the second anticancer agent inhibits PLK. In some embodiments, the second anticancer agent is selected from BI-2536 and GSK461364. In some embodiments, the second anticancer agent inhibits DNA methyltransferase. In some embodiments, the second anticancer agent is azacitidine. In some embodiments, the second anticancer agent inhibits the Ras/MAPK pathway. In some embodiments, the second anticancer agent is selected from sorafenib and PLX-4032. In some embodiments, the second anticancer agent inhibits FGFR1 tyrosine kinase. In some embodiments, the second anticancer agent is JNJ-13.

In some embodiments, the second anticancer agent is selected from AZD0503, dasatinib and nilotinib, and JNJ-20, wherein the combination provides a synergistic therapeutic effect compared to administration of ibrutinib or the second anticancer agent alone. In some embodiments, the second anticancer agent is AZD0503. In some embodiments, the second anticancer agent is dasatinib. In some embodiments, the second anticancer agent is nilotinib. In some embodiments, the second anticancer agent is JNJ-20.

In some embodiments, Ibrutinib and a second anticancer agent are co-administered concurrently (e.g., simultaneously, essentially simultaneously or within the same treatment protocol) or sequentially.

In some embodiments, Ibrutinib and a second anticancer agentare co-administered in separate dosage forms. In some embodiments, Ibrutinib and a second anticancer agent are co-administered in combined dosage forms.

In some embodiments, the co-administration of Ibrutinib and a second anticancer agent increases the oral bioavailability of Ibrutinib. In some embodiments, the co-administration of Ibrutinib and a second anticancer agent increases the Cmax of Ibrutinib. In some embodiments, the co-administration of Ibrutinib and a second anticancer agent increases the AUC of Ibrutinib.

In some embodiments, co-administration of Ibrutinib and a second anticancer agent increases the Cmax of Ibrutinib by about 20× to about 40× the Cmax of Ibrutinib administered without a second anticancer agent. In some embodiments, co-administration of Ibrutinib and a second anticancer agent increases the Cmax of Ibrutinib by about 25× to about 35×. In some embodiments, co-administration of Ibrutinib and a second anticancer agent increases the Cmax of Ibrutinib by about 20×. In some embodiments, co-administration of Ibrutinib and a second anticancer agent increases the Cmax of Ibrutinib by about 21×. In some embodiments, co-administration of Ibrutinib and a second anticancer agent increases the Cmax of Ibrutinib by about 22×. In some embodiments, co-administration of Ibrutinib and a second anticancer agent increases the Cmax of Ibrutinib by about 23×. In some embodiments, co-administration of Ibrutinib and a second anticancer agent increases the Cmax of Ibrutinib by about 24×. In some embodiments, co-administration of Ibrutinib and a Second anticancer agent increases the Cmax of Ibrutinib by about 25×. In some embodiments, co-administration of Ibrutinib and a Second anticancer agent increases the Cmax of Ibrutinib by about 26×. In some embodiments, co-administration of Ibrutinib and a Second anticancer agent increases the Cmax of Ibrutinib by about 27×. In some embodiments, co-administration of Ibrutinib and a Second anticancer agent increases the Cmax of Ibrutinib by about 28×. In some embodiments, co-administration of Ibrutinib and a Second anticancer agent increases the Cmax of Ibrutinib by about 29×. In some embodiments, co-administration of Ibrutinib and a Second anticancer agent increases the Cmax of Ibrutinib by about 30×. In some embodiments, co-administration of Ibrutinib and a Second anticancer agent increases the Cmax of Ibrutinib by about 31×. In some embodiments, co-administration of Ibrutinib and a Second anticancer agent increases the Cmax of Ibrutinib by about 32×. In some embodiments, co-administration of Ibrutinib and a Second anticancer agent increases the Cmax of Ibrutinib by about 33×. In some embodiments, co-administration of Ibrutinib and a Second anticancer agent increases the Cmax of Ibrutinib by about 34×. In some embodiments, co-administration of Ibrutinib and a Second anticancer agent increases the Cmax of Ibrutinib by about 35×. In some embodiments, co-administration of Ibrutinib and a Second anticancer agent increases the Cmax of Ibrutinib by about 36×. In some embodiments, co-administration of Ibrutinib and a Second anticancer agent increases the Cmax of Ibrutinib by about 37×. In some embodiments, co-administration of Ibrutinib and a Second anticancer agent increases the Cmax of Ibrutinib by about 38×. In some embodiments, co-administration of Ibrutinib and a Second anticancer agent increases the Cmax of Ibrutinib by about 39×. In some embodiments, co-administration of Ibrutinib and a Second anticancer agent increases the Cmax of Ibrutinib by about 40×.

In some embodiments, the co-administration of Ibrutinib and a Second anticancer agent increases the AUC of Ibrutinib by about 15× to about 35× the AUC of Ibrutinib administered without a Second anticancer agent. In some embodiments, co-administration of Ibrutinib and a Second anticancer agent increases the AUC of Ibrutinib by about 20× to about 30×. In some embodiments, co-administration of Ibrutinib and a Second anticancer agent increases the AUC of Ibrutinib by about 20× to about 35× the AUC of Ibrutinib administered without a Second anticancer agent. In some embodiments, co-administration of Ibrutinib and a Second anticancer agent increases the AUC of Ibrutinib by about 20× to about 30× the AUC of Ibrutinib administered without a Second anticancer agent. In some embodiments, co-administration of Ibrutinib and a Second anticancer agent increases the AUC of Ibrutinib by about 20× to about 25× the AUC of Ibrutinib administered without a Second anticancer agent. In some embodiments, co-administration of Ibrutinib and a Second anticancer agent increases the AUC of Ibrutinib by about 2× to about 20× the AUC of Ibrutinib administered without a Second anticancer agent. In some embodiments, co-administration of Ibrutinib and a Second anticancer agent increases the AUC of Ibrutinib by about 2× to about 15× the AUC of Ibrutinib administered without a Second anticancer agent. In some embodiments, co-administration of Ibrutinib and a Second anticancer agent increases the AUC of Ibrutinib by about 2× to about 10× the AUC of Ibrutinib administered without a Second anticancer agent. In some embodiments, co-administration of Ibrutinib and a Second anticancer agent increases the AUC of Ibrutinib by about 2× to about 5× the AUC of Ibrutinib administered without a Second anticancer agent. In some embodiments, co-administration of Ibrutinib and a Second anticancer agent increases the AUC of Ibrutinib by about 2× to about 4× the AUC of Ibrutinib administered without a Second anticancer agent. In some embodiments, co-administration of Ibrutinib and a Second anticancer agent increases the AUC of Ibrutinib by about 15×. In some embodiments, co-administration of Ibrutinib and a Second anticancer agent increases the AUC of Ibrutinib by about 2×. In some embodiments, co-administration of Ibrutinib and a Second anticancer agent increases the AUC of Ibrutinib by about 3×. In some embodiments, co-administration of Ibrutinib and a Second anticancer agent increases the AUC of Ibrutinib by about 4×. In some embodiments, co-administration of Ibrutinib and a Second anticancer agent increases the AUC of Ibrutinib by about 5×. In some embodiments, co-administration of Ibrutinib and a Second anticancer agent increases the AUC of Ibrutinib by about 6×. In some embodiments, co-administration of Ibrutinib and a Second anticancer agent increases the AUC of Ibrutinib by about 7×. In some embodiments, co-administration of Ibrutinib and a Second anticancer agent increases the AUC of Ibrutinib by about 8×. In some embodiments, co-administration of Ibrutinib and a Second anticancer agent increases the AUC of Ibrutinib by about 9×. In some embodiments, co-administration of Ibrutinib and a Second anticancer agent increases the AUC of Ibrutinib by about 10×. In some embodiments, co-administration of Ibrutinib and a Second anticancer agent increases the AUC of Ibrutinib by about 11×. In some embodiments, co-administration of Ibrutinib and a Second anticancer agent increases the AUC of Ibrutinib by about 12×. In some embodiments, co-administration of Ibrutinib and a Second anticancer agent increases the AUC of Ibrutinib by about 13×. In some embodiments, co-administration of Ibrutinib and a Second anticancer agent increases the AUC of Ibrutinib by about 14×. In some embodiments, co-administration of Ibrutinib and a Second anticancer agent increases the AUC of Ibrutinib by about 15×. In some embodiments, co-administration of Ibrutinib and a Second anticancer agent increases the AUC of Ibrutinib by about 16×. In some embodiments, co-administration of Ibrutinib and a Second anticancer agent increases the AUC of Ibrutinib by about 17×. In some embodiments, co-administration of Ibrutinib and a Second anticancer agent increases the AUC of Ibrutinib by about 18×. In some embodiments, co-administration of Ibrutinib and a Second anticancer agent increases the AUC of Ibrutinib by about 19×. In some embodiments, co-administration of Ibrutinib and a Second anticancer agent increases the AUC of Ibrutinib by about 20×. In some embodiments, co-administration of Ibrutinib and a Second anticancer agent increases the AUC of Ibrutinib by about 21×. In some embodiments, co-administration of Ibrutinib and a Second anticancer agent increases the AUC of Ibrutinib by about 22×. In some embodiments, co-administration of Ibrutinib and a Second anticancer agent increases the AUC of Ibrutinib by about 23×. In some embodiments, co-administration of Ibrutinib and a Second anticancer agent increases the AUC of Ibrutinib by about 24×. In some embodiments, co-administration of Ibrutinib and a Second anticancer agent increases the AUC of Ibrutinib by about 25×. In some embodiments, co-administration of Ibrutinib and a Second anticancer agent increases the AUC of Ibrutinib by about 26×. In some embodiments, co-administration of Ibrutinib and a Second anticancer agent increases the AUC of Ibrutinib by about 27×. In some embodiments, co-administration of Ibrutinib and a Second anticancer agent increases the AUC of Ibrutinib by about 28×. In some embodiments, co-administration of Ibrutinib and a Second anticancer agent increases the AUC of Ibrutinib by about 29×. In some embodiments, co-administration of Ibrutinib and a Second anticancer agent increases the AUC of Ibrutinib by about 30×. In some embodiments, co-administration of Ibrutinib and a Second anticancer agent increases the AUC of Ibrutinib by about 31×. In some embodiments, co-administration of Ibrutinib and a Second anticancer agent increases the AUC of Ibrutinib by about 32×. In some embodiments, co-administration of Ibrutinib and a Second anticancer agent increases the AUC of Ibrutinib by about 33×. In some embodiments, co-administration of Ibrutinib and a Second anticancer agent increases the AUC of Ibrutinib by about 34×. In some embodiments, co-administration of Ibrutinib and a Second anticancer agent increases the AUC of Ibrutinib by about 35×.

In some embodiments, co-administration of Ibrutinib and a Second anticancer agent does not significantly affect the Tmax or T1/2 of Ibrutinib as compared to the Tmax and T1/2 of Ibrutinib administered without a Second anticancer agent.

In some embodiments, the daily dosage of Ibrutinib when administered in combination with a Second anticancer agent is about 10 mg to about 140 mg. In some embodiments, the daily dosage of Ibrutinib when administered in combination with a Second anticancer agent is less than about 10 mg. In some embodiments, the daily dosage of Ibrutinib when administered in combination with a Second anticancer agent is greater than about 140 mg In some embodiments, the daily dosage of Ibrutinib when administered in combination with a Second anticancer agent is about 10, mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the daily dosage of Ibrutinib when administered in combination with a Second anticancer agent is about 40 mg to about 70 mg. In some embodiments, the daily dosage of Ibrutinib when administered in combination with a Second anticancer agent is about 40 mg.

Any suitable daily dose of a Second anticancer agent is contemplated for use with the compositions, dosage forms, and methods disclosed herein. Daily dose of the Second anticancer agent depends on multiple factors, the determination of which is within the skills of one of skill in the art. For example, the daily dose of the Second anticancer agent depends of the strength of the Second anticancer agent. Weak Second anticancer agents will require higher daily doses than moderate Second anticancer agents, and moderate Second anticancer agents will require higher daily doses than strong Second anticancer agents.

### Exemplary Second Anticancer Agents

In some embodiments, the second anticancer agent inhibits Bcl-2; Janus kinase 2 (JAK2); Anaplastic lymphoma kinase (ALK); or heat shock protein 90 (Hsp90), wherein the combination provides a synergistic therapeutic effect compared to administration of ibrutinib or the second anticancer agent alone. In some embodiments, the second anticancer agent inhibits Bcl-2. In some embodiments, the second anticancer agent that inhibits Bcl-2 is selected from ABT-737, ABT-199 and HA14-1. In some embodiments, the second anticancer agent inhibits JAK2. In some embodiments, the second anticancer agent that inhibits JAK2 is TG-101348. In some embodiments, the second anticancer agent inhibits ALK. In some embodiments, the second anticancer agent that inhibits ALK is NVP-TAE684. In some embodiments, the second anticancer agent inhibits Hsp90. In some embodiments, the second anticancer agent that inhibits Hsp 90 is 17-DMAG.

In some embodiments, the second anticancer agent is a glucocorticoid, a vinca alkaloid, an anti-metabolite, a DNA damaging agent, lenalidomide, rituximab, or a PKC perturbagen, wherein the combination provides a synergistic therapeutic effect compared to administration of ibrutinib or the second anticancer agent alone. In some embodiments, the second anticancer agent is a glucocorticoid. In some embodiments, the second anticancer agent is selected from dexamethasone and prednisolone. In some embodiments, the second anticancer agent is a vinca alkaloid. In some embodiments, the second anticancer agent is vincristine. In some embodiments, the second anticancer agent is an anti-metabolite. In some embodiments, the second anticancer agent is gemcitabine. In some embodiments, the second anticancer agent is a DNA damaging agent. In some embodiments, the DNA damaging agent is selected from carboplatin and chlorambucil. In some embodiments, the second anticancer agent is lenalidomide. In some embodiments, the second anticancer agent is rituximab. In some embodiments, the second anticancer agent is a PKC perturbagen. In some embodiments, the PKC perturbagen is selected from enzastarin and GF109203X.

In some embodiments, the second anticancer agent inhibits a B-cell receptor pathway kinase selected from among Lyn/Fyn, Syk, PI3K, PKCβ, and IKK, wherein the combination provides a synergistic therapeutic effect compared to administration of ibrutinib or the second anticancer agent alone. In some embodiments, the second anticancer agent inhibits a B-cell receptor pathway kinase selected from among Lyn/Fyn, Syk, PI3K, PKCβ, and IKK. In some embodiments, the second anticancer agent inhibits Lyn/Fyn. In some embodiments, the second anticancer agent inhibits Syk. In some embodiments, the second anticancer agent is R406. In some embodiments, the second anticancer agent inhibits PKCβ. In some embodiments, the second anticancer agent inhibits IKK. In some embodiments, the second anticancer agent inhibits PI3K. In some embodiments, the second anticancer agent that inhibits PI3K is selected from IPI-145, BKM120, BEZ235, GDC-0941, AMG319, CAL-101 and A66.

In some embodiments, the second anticancer agent inhibits the 20s proteasome, IRF-4, IRAK4, EZH2, CXCR4, CXCR5, GLS, cyclin dependent kinase 4/6 (CDK4/6), topoisomerase II, PLK; DNA methyltransferase, the Ras/MAPK pathway, or FGFR1 tyrosine kinase, wherein the combination provides a synergistic therapeutic effect compared to administration of ibrutinib or the second anticancer agent alone. In some embodiments, the second anticancer agent inhibits the 20s proteasome. In some embodiments, the second anticancer agent is carfilzomib. In some embodiments, the second anticancer agent inhibits IRF-4. In some embodiments, the second anticancer agent is LEN. In some embodiments, the second anticancer agent inhibits IRAK4. In some embodiments, the second anticancer agent is ND-2158. In some embodiments, the second anticancer agent inhibits EZH2. In some embodiments, the second anticancer agent is selected from EI1, GSK343 and EPZ005687. In some embodiments, the second anticancer agent inhibits CXCR4. In some embodiments, the second anticancer agent is AMD3100. In some embodiments, the second anticancer agent inhibits CXCR5. In some embodiments, the second anticancer agent is an antibody against CXCR5. In some embodiments, wherein the second anticancer agent inhibits GLS. In some embodiments, the second anticancer agent is JNJ-16. In some embodiments, wherein the second anticancer agent inhibits CDK4/6. In some embodiments, the second anticancer agent is JNJ-08. In some embodiments, the second anticancer agent inhibits topoisomerase II. In some embodiments, the second anticancer agent is selected from doxorubicin and etoposide. In some embodiments, the second anticancer agent inhibits PLK. In some embodiments, the second anticancer agent is selected from BI-2536 and GSK461364. In some embodiments, the second anticancer agent inhibits DNA methyltransferase. In some embodiments, the second anticancer agent is azacitidine. In some embodiments, the second anticancer agent inhibits the Ras/MAPK pathway. In some embodiments, the second anticancer agent is selected from sorafenib and PLX-4032. In some embodiments, the second anticancer agent inhibits FGFR1 tyrosine kinase. In some embodiments, the second anticancer agent is JNJ-13.

In some embodiments, the second anticancer agent is selected from AZD0503, dasatinib and nilotinib, and JNJ-20, wherein the combination provides a synergistic therapeutic effect compared to administration of ibrutinib or the second anticancer agent alone. In some embodiments, the second anticancer agent is AZD0503. In some embodiments, the second anticancer agent is dasatinib. In some embodiments, the second anticancer agent is nilotinib. In some embodiments, the second anticancer agent is JNJ-20.

Any suitable Second anticancer agent is contemplated for use with the compositions, dosage forms, and methods disclosed herein. The selection of the Second anticancer agent depends on multiple factors, and the selection of the Second anticancer agent is within the skills of one of skill in the art. For example, factors to be considered include the desired reduction in the daily dose of Ibrutinib, any additional drug interactions of the Second anticancer agent, and the length for which the Second anticancer agent may be taken. In certain instances, the Second anticancer agent is a Second anticancer agent which may be taken long-term, for example chronically.

Disclosed herein, in certain embodiments, are methods of increasing the Cmax of ibruitinib comprising co-administering a combination of Ibrutinib and a Second anticancer agent. In some embodiments, Cmax of Ibrutinib is increased by about 20× to about 40× the Cmax of Ibrutinib administered without a Second anticancer agent, or about 25× to about 35×. In some embodiments, the method increases the AUC of Ibrutinib. In some embodiments, the method increases the AUC of Ibrutinib by about 15× to about 35× the AUC of Ibrutinib administered without a Second anticancer agent, or about 20× to about 30×. In some embodiments, the method increases the AUC of Ibrutinib by about 2× to about 35× the AUC of Ibrutinib administered without a Second anticancer agent. In some embodiments, the method increases the AUC of Ibrutinib by about 2× to about 30× the AUC of Ibrutinib administered without a Second anticancer agent. In some embodiments, the method increases the AUC of Ibrutinib by about 2× to about 25× the AUC of Ibrutinib administered without a Second anticancer agent. In some embodiments, the method increases the AUC of Ibrutinib by about 2× to about 20× the AUC of Ibrutinib administered without a Second anticancer agent. In some embodiments, the method increases the AUC of Ibrutinib by about 2× to about 15× the AUC of Ibrutinib administered without a Second anticancer agent. In some embodiments, the method increases the AUC of Ibrutinib by about 2× to about 10× the AUC of Ibrutinib administered without a Second anticancer agent. In some embodiments, the method increases the AUC of Ibrutinib by about 2× to about 5× the AUC of Ibrutinib administered without a Second anticancer agent. In some embodiments, the method increases the AUC of Ibrutinib by about 2× to about 4× the AUC of Ibrutinib administered without a Second anticancer agent. In some embodiments, the method does not significantly affect the Tmax or T1/2 of Ibrutinib as compared to the Tmax and T1/2 of Ibrutinib administered without a Second anticancer agent.

Disclosed herein, in certain embodiments, are methods of increasing the AUC of Ibrutinib comprising administering a combination of Ibrutinib and a Second anticancer agent. In some embodiments, the method increases the AUC of Ibrutinib by about 15× to about 35× the AUC of Ibrutinib administered without a Second anticancer agent, or about 20× to about 30×. In some embodiments, the method increases the AUC of Ibrutinib by about 2× to about 35× the AUC of Ibrutinib administered without a Second anticancer agent. In some embodiments, the method increases the AUC of Ibrutinib by about 2× to about 30× the AUC of Ibrutinib administered without a Second anticancer agent. In some embodiments, the method increases the AUC of Ibrutinib by about 2× to about 25× the AUC of Ibrutinib administered without a Second anticancer agent. In some embodiments, the method increases the AUC of Ibrutinib by about 2× to about 20× the AUC of Ibrutinib administered without a Second anticancer agent. In some embodiments, the method increases the AUC of Ibrutinib by about 2× to about 15× the AUC of Ibrutinib administered without a Second anticancer agent. In some embodiments, the method increases the AUC of Ibrutinib by about 2× to about 10× the AUC of Ibrutinib administered without a Second anticancer agent. In some embodiments, the method increases the AUC of Ibrutinib by about 2× to about 5× the AUC of Ibrutinib administered without a Second anticancer agent. In some embodiments, the method increases the AUC of Ibrutinib by about 2× to about 4× the AUC of Ibrutinib administered without a Second anticancer agent. In some embodiments, the method increases the Cmax of Ibrutinib. In some embodiments, Cmax of Ibrutinib is increased by about 20× to about 40× the Cmax of Ibrutinib administered without a Second anticancer agent, or about 25× to about 35×. In some embodiments, the method does not significantly affect the Tmax or T1/2 of Ibrutinib as compared to the Tmax and T1/2 of Ibrutinib administered without a Second anticancer agent.

### Methods of Use

In some embodiments is a method of treating a cancer in an individual in need thereof comprising administering a combination of a Btk inhibitor and a Second anticancer agent. In some embodiments, the cancer comprises a tumor. In some embodiments, the tumor is a sarcoma, carcinoma, neurofibromatoma or a lymphoma. In some embodiments, the lymphoma is an enlarged lymph node or an extranodal lymphoma. In some embodiments, the subject has a brain, breast, bladder, bone, colon, kidney, liver, lung, ovarian, pancreatic, prostate, skin or proximal or distal bile duct carcinoma. In some embodiments, the subject has a hematologic cancer. In some embodiments, the cancer is a lymphoma. In some embodiments, the subject has a non-Hodgkin's lymphoma. In some embodiments, the non-Hodgkin's lymphoma is chronic lymphocytic leukemia/small lymphocytic lymphoma (CLL/SLL), follicular lymphoma (FL), diffuse large B-cell lymphoma (DLBCL), mantle cell lymphoma (MCL), Waldenstrom's macroglobulinemia, multiple myeloma, marginal zone lymphoma, Burkitt's lymphoma, non-Burkitt high grade B cell lymphoma, or extranodal marginal zone B cell lymphoma. In some embodiments, the non-Hodgkin's lymphoma is a relapsed or refractory non-Hodgkin's lymphoma. In some embodiments, the subject has a T-cell malignancy. In some embodiments, the T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some embodiments, the subject has a bladder, brain, breast, bladder, bone, cervical, colon, esophageal, kidney, liver, lung, ovarian, pancreatic, proximal or distal bile duct, prostate, skin, stomach, thyroid, or uterine cancer. In some embodiments, the subject has a metastatic cancer. In some embodiments, the subject has a cancer that is acute lymphoblastic leukemia, acute lymphoblastic leukemia, acute myeloid leukemia, acute promyelocytic leukemia, adenocarcinoma, adenoma, adrenal cancer, adrenocortical carcinoma, AIDS-related cancer, AIDS-related lymphoma, anal cancer, appendix cancer, astrocytoma, basal cell carcinoma, bile duct cancer, bladder cancer, bone cancer, osteosarcoma/malignant fibrous histiocytoma, brainstem glioma, brain cancer, carcinoma, cerebellar astrocytoma, cerebral astrocytoma/malignant glioma, ependymoma, medulloblastoma, supratentorial primitive neuroectodermal tumor, visual pathway or hypothalamic glioma, breast cancer, bronchial adenoma/carcinoid, Burkitt lymphoma, carcinoid tumor, carcinoma, central nervous system lymphoma, cervical cancer, chronic lymphocytic leukemia, chronic myelogenous leukemia, chronic myeloproliferative disorder, colon cancer, cutaneous T-cell lymphoma, desmoplastic small round cell tumor, endometrial cancer, ependymoma. epidermoid carcinoma, esophageal cancer, Ewing's sarcoma, extracranial germ cell tumor, extragonadal germ cell tumor, extrahepatic bile duct cancer, eye cancer/intraocular melanoma, eye cancer/retinoblastoma, gallbladder cancer, gallstone tumor, gastric/stomach cancer, gastrointestinal carcinoid tumor, gastrointestinal stromal tumor, giant cell tumor, glioblastoma multiforme, glioma, hairy-cell tumor, head and neck cancer, heart cancer, hepatocellular/liver cancer, Hodgkin lymphoma, hyperplasia, hyperplastic corneal nerve tumor, in situ carcinoma, hypopharyngeal cancer, intestinal ganglioneuroma, islet cell tumor, Kaposi's sarcoma, kidney/renal cell cancer, laryngeal cancer, leiomyoma tumor, lip and oral cavity cancer, liposarcoma, liver cancer, non-small cell lung cancer, small cell lung cancer, lymphomas, macroglobulinemia, malignant carcinoid, malignant fibrous histiocytoma of bone, malignant hypercalcemia, malignant melanomas, marfanoid habitus tumor, medullary carcinoma, melanoma, merkel cell carcinoma, mesothelioma, metastatic skin carcinoma, metastatic squamous neck cancer, mouth cancer, mucosal neuromas, multiple myeloma, mycosis fungoides, myelodysplastic syndrome, myeloma, myeloproliferative disorder, nasal cavity and paranasal sinus cancer, nasopharyngeal carcinoma, neck cancer, neural tissue cancer, neuroblastoma, oral cancer, oropharyngeal cancer, osteosarcoma, ovarian cancer, ovarian epithelial tumor, ovarian germ cell tumor, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, polycythemia vera, primary brain tumor, prostate cancer, rectal cancer, renal cell tumor, reticulum cell sarcoma, retinoblastoma, rhabdomyosarcoma, salivary gland cancer, seminoma, Sezary syndrome, skin cancer, small intestine cancer, soft tissue sarcoma, squamous cell carcinoma, squamous neck carcinoma, stomach cancer, supratentorial primitive neuroectodermal tumor, testicular cancer, throat cancer, thymoma, thyroid cancer, topical skin lesion, trophoblastic tumor, urethral cancer, uterine/endometrial cancer, uterine sarcoma, vaginal cancer, vulvar cancer, Waldenström's macroglobulinemia or Wilm's tumor.

In some embodiments, the subject has a solid tumor. In some embodiments, the subject has a sarcoma, carcinoma, a neurofibromatoma or a lymphoma. In some embodiments, the subject has a colon cancer. In some embodiments, the subject has a lung cancer. In some embodiments, the subject has an ovarian cancer. In some embodiments, the subject has a pancreatic cancer. In some embodiments, the subject has a prostate cancer. In some embodiments, the subject has a proximal or distal bile duct carcinoma. In some embodiments, the subject has a breast cancer. In some embodiments, the subject has a HER2-positive breast cancer. In some embodiments, the subject has a HER2-negative breast cancer.

In some embodiments, the cancer is a hematologic cancer. In some embodiments, cancer is a leukemia, a lymphoma, or a myeloma. In some embodiments, cancer is a non-Hodgkin lymphoma. In some embodiments, cancer is a Hodgkin lymphoma.

In some embodiments, the cancer is a T-cell malignancy. In some embodiments, the T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas. In some embodiments, the subject has multiple myeloma.

In some embodiments, the subject has a relapsed or refractory cancer. In some embodiments, the relapsed or refractory cancer is a bladder cancer. In some embodiments, the relapsed or refractory cancer is a colon cancer. In some embodiments, the relapsed or refractory cancer is a lung cancer. In some embodiments, the relapsed or refractory cancer is an ovarian cancer. In some embodiments, the relapsed or refractory cancer is a pancreatic cancer. In some embodiments, the relapsed or refractory cancer is a prostate cancer. In some embodiments, the relapsed or refractory cancer is a proximal or distal bile duct carcinoma. In some embodiments, the relapsed or refractory cancer is a breast cancer.

In some embodiments, the subject has a relapsed or refractory hematologic cancer. In some embodiments, the relapsed or refractory hematologic cancer is a leukemia, a lymphoma, or a myeloma. In some embodiments, the relapsed or refractory hematologic cancer is a non-Hodgkin lymphoma. In some embodiments, the relapsed or refractory hematologic cancer is a Hodgkin lymphoma. In some embodiments, the relapsed or refractory hematologic cancer is a B-cell malignancy. In some embodiments, the B-cell malignancy is chronic lymphocytic leukemia (CLL), small lymphocytic lymphoma (SLL), diffuse large B-cell lymphoma (DLBCL), follicular lymphoma (FL), activated B-cell diffuse large B-cell lymphoma (ABC-DLBCL), germinal center diffuse large B-cell lymphoma (GCB DLBCL), primary mediastinal B-cell lymphoma (PMBL), Burkitt's lymphoma, immunoblastic large cell lymphoma, precursor B-lymphoblastic lymphoma, mantle cell lymphoma (MCL), B cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, nodal marginal zone B cell lymphoma, mediastinal (thymic) large B cell lymphoma, intravascular large B cell lymphoma, primary effusion lymphoma, or lymphomatoid granulomatosis. In some embodiments, the relapsed or refractory hematologic cancer is a T-cell malignancy. In some embodiments, the T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas. In some embodiments, the subject has a relapsed or refractory multiple myeloma. In some embodiments, the regression of a relapsed or refractory cancer ceases.

### B-Cell Proliferative Disorders

In some embodiments is a method of treating a cancer in an individual in need thereof comprising administering a combination of a Btk inhibitor and a Second anticancer agent. In some embodiments, the cancer is a B-cell proliferative disorder.

Disclosed herein, in some embodiments, is a method for treating a B-cell proliferative disorder comprising administering to a subject in need thereof a therapeutically effective amount of a combination comprising: a. a therapeutically effective amount of Ibrutinib; b. a second anticancer agent, wherein the second anticancer agent inhibits Bcl-2; Janus kinase 2 (JAK2); Anaplastic lymphoma kinase (ALK); or heat shock protein 90 (Hsp90), wherein the combination provides a synergistic therapeutic effect compared to administration of ibrutinib or the second anticancer agent alone. In some embodiments, the second anticancer agent inhibits Bcl-2. In some embodiments, the second anticancer agent that inhibits Bcl-2 is selected from ABT-737, ABT-199 and HA14-1. In some embodiments, the second anticancer agent inhibits JAK2. In some embodiments, the second anticancer agent that inhibits JAK2 is TG-101348. In some embodiments, the second anticancer agent inhibits ALK. In some embodiments, the second anticancer agent that inhibits ALK is NVP-TAE684. In some embodiments, the second anticancer agent inhibits Hsp90. In some embodiments, the second anticancer agent that inhibits Hsp 90 is 17-DMAG. In some embodiments, the B-cell proliferative disorder is diffuse large B-cell lymphoma (DLBCL), chronic lymphocytic leukemia (CLL), small lymphocytic lymphoma (SLL), high risk CLL, or a non-CLL/SLL lymphoma, follicular lymphoma, mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma, acute or chronic myelogenous (or myeloid) leukemia, myelodysplastic syndrome, or acute lymphoblastic leukemia. In some embodiments, the B-cell proliferative disorder is DLBCL. In some embodiments, the DLBCL is “activated B-cell” (ABC) DLBCL. In some embodiments, the DLBCL is “germinal center B-cell like” (GCB) DLBCL.

Disclosed herein, in some embodiments, is a method for treating a B-cell proliferative disorder comprising administering to a subject in need thereof a therapeutically effective amount of a combination comprising: a. a therapeutically effective amount of Ibrutinib; b. a second anticancer agent wherein the second anticancer agent is a glucocorticoid, a vinca alkaloid, an anti-metabolite, a DNA damaging agent, lenalidomide, rituximab, or a PKC perturbagen, wherein the combination provides a synergistic therapeutic effect compared to administration of ibrutinib or the second anticancer agent alone. In some embodiments, the second anticancer agent is a glucocorticoid. In some embodiments, the second anticancer agent is selected from dexamethasone and prednisolone. In some embodiments, the second anticancer agent is a vinca alkaloid. In some embodiments, the second anticancer agent is vincristine. In some embodiments, the second anticancer agent is an anti-metabolite. In some embodiments, the second anticancer agent is gemcitabine. In some embodiments, the second anticancer agent is a DNA damaging agent. In some embodiments, the DNA damaging agent is selected from carboplatin and chlorambucil. In some embodiments, the second anticancer agent is lenalidomide. In some embodiments, the second anticancer agent is rituximab. In some embodiments, the second anticancer agent is a PKC perturbagen. In some embodiments, the PKC perturbagen is selected from enzastarin and GF109203X. In some embodiments, the B-cell proliferative disorder is diffuse large B-cell lymphoma (DLBCL), chronic lymphocytic leukemia (CLL), small lymphocytic lymphoma (SLL), high risk CLL, or a non-CLL/SLL lymphoma, follicular lymphoma, mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma, acute or chronic myelogenous (or myeloid) leukemia, myelodysplastic syndrome, or acute lymphoblastic leukemia. In some embodiments, the B-cell proliferative disorder is DLBCL. In some embodiments, the DLBCL is “activated B-cell” (ABC) DLBCL. In some embodiments, the DLBCL is “germinal center B-cell like” (GCB) DLBCL.

Disclosed herein, in some embodiments, is a method for treating a B-cell proliferative disorder comprising administering to a subject in need thereof a therapeutically effective amount of a combination comprising: a. Ibrutinib; and b. a second anticancer agent, wherein the second anticancer agent inhibits a B-cell receptor pathway kinase selected from among Lyn/Fyn, Syk, PI3K, PKCβ, and IKK, wherein the combination provides a synergistic therapeutic effect compared to administration of ibrutinib or the second anticancer agent alone. In some embodiments, the second anticancer agent inhibits a B-cell receptor pathway kinase selected from among Lyn/Fyn, Syk, PI3K, PKCβ, and IKK. In some embodiments, the second anticancer agent inhibits Lyn/Fyn. In some embodiments, the second anticancer agent inhibits Syk. In some embodiments, the second anticancer agent is R406. In some embodiments, the second anticancer agent inhibits PKCβ. In some embodiments, the second anticancer agent inhibits IKK. In some embodiments, the second anticancer agent inhibits PI3K. In some embodiments, the second anticancer agent that inhibits PI3K is selected from IPI-145, BKM120, BEZ235, GDC-0941, AMG319, CAL-101 and A66. In some embodiments, the B-cell proliferative disorder is diffuse large B-cell lymphoma (DLBCL), chronic lymphocytic leukemia (CLL), small lymphocytic lymphoma (SLL), high risk CLL, or a non-CLL/SLL lymphoma, follicular lymphoma, mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma, acute or chronic myelogenous (or myeloid) leukemia, myelodysplastic syndrome, or acute lymphoblastic leukemia. In some embodiments, the B-cell proliferative disorder is DLBCL. In some embodiments, the DLBCL is “activated B-cell” (ABC) DLBCL. In some embodiments, the DLBCL is “germinal center B-cell like” (GCB) DLBCL.

Disclosed herein, in some embodiments, is a method for treating a B-cell proliferative disorder comprising administering to a subject in need thereof a therapeutically effective amount of a combination comprising: a. a therapeutically effective amount of Ibrutinib; and b. a second anticancer agent, wherein the second anticancer agent inhibits the 20s proteasome, IRF-4, IRAK4, EZH2, CXCR4, CXCR5, GLS, cyclin dependent kinase 4/6 (CDK4/6), topoisomerase II, PLK; DNA methyltransferase, the Ras/MAPK pathway, or FGFR1 tyrosine kinase, wherein the combination provides a synergistic therapeutic effect compared to administration of ibrutinib or the second anticancer agent alone. In some embodiments, the second anticancer agent inhibits the 20s proteasome. In some embodiments, the second anticancer agent is carfilzomib. In some embodiments, the second anticancer agent inhibits IRF-4. In some embodiments, the second anticancer agent is LEN. In some embodiments, the second anticancer agent inhibits IRAK4. In some embodiments, the second anticancer agent is ND-2158. In some embodiments, the second anticancer agent inhibits EZH2. In some embodiments, the second anticancer agent is selected from EI1, GSK343 and EPZ005687. In some embodiments, wherein the second anticancer agent inhibits CXCR4. In some embodiments, the second anticancer agent is AMD3100. In some embodiments, the second anticancer agent inhibits CXCR5. In some embodiments, the second anticancer agent is an antibody against CXCR5. In some embodiments, wherein the second anticancer agent inhibits GLS. In some embodiments, the second anticancer agent is JNJ-16. In some embodiments, wherein the second anticancer agent inhibits CDK4/6. In some embodiments, the second anticancer agent is JNJ-08. In some embodiments, the second anticancer agent inhibits topoisomerase II. In some embodiments, the second anticancer agent is selected from doxorubicin and etoposide. In some embodiments, the second anticancer agent inhibits PLK. In some embodiments, the second anticancer agent is selected from BI-2536 and GSK461364. In some embodiments, the second anticancer agent inhibits DNA methyltransferase. In some embodiments, the second anticancer agent is azacitidine. In some embodiments, the second anticancer agent inhibits the Ras/MAPK pathway. In some embodiments, the second anticancer agent is selected from sorafenib and PLX-4032. In some embodiments, the second anticancer agent inhibits FGFR1 tyrosine kinase. In some embodiments, the second anticancer agent is JNJ-13. In some embodiments, the B-cell proliferative disorder is diffuse large B-cell lymphoma (DLBCL), chronic lymphocytic leukemia (CLL), small lymphocytic lymphoma (SLL), high risk CLL, or a non-CLL/SLL lymphoma, follicular lymphoma, mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma, acute or chronic myelogenous (or myeloid) leukemia, myelodysplastic syndrome, or acute lymphoblastic leukemia. In some embodiments, the B-cell proliferative disorder is DLBCL. In some embodiments, the DLBCL is “activated B-cell” (ABC) DLBCL. In some embodiments, the DLBCL is “germinal center B-cell like” (GCB) DLBCL.

Disclosed herein, in some embodiments, is a method for treating a B-cell proliferative disorder comprising administering to a subject in need thereof a therapeutically effective amount of a combination comprising: a. a therapeutically effective amount of Ibrutinib; and b. a second anticancer agent, wherein the second anticancer agent is selected from AZD0503, dasatinib and nilotinib, and JNJ-20, wherein the combination provides a synergistic therapeutic effect compared to administration of ibrutinib or the second anticancer agent alone. In some embodiments, the second anticancer agent is AZD0503. In some embodiments, the second anticancer agent is dasatinib. In some embodiments, the second anticancer agent is nilotinib. In some embodiments, the second anticancer agent is JNJ-20. In some embodiments, the B-cell proliferative disorder is diffuse large B-cell lymphoma (DLBCL), chronic lymphocytic leukemia (CLL), small lymphocytic lymphoma (SLL), high risk CLL, or a non-CLL/SLL lymphoma, follicular lymphoma, mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma, acute or chronic myelogenous (or myeloid) leukemia, myelodysplastic syndrome, or acute lymphoblastic leukemia. In some embodiments, the B-cell proliferative disorder is DLBCL. In some embodiments, the DLBCL is “activated B-cell” (ABC) DLBCL. In some embodiments, the DLBCL is “germinal center B-cell like” (GCB) DLBCL.

In some embodiments, the cancer is chronic lymphocytic leukemia (CLL), small lymphocytic lymphoma (SLL), high risk CLL, or a non-CLL/SLL lymphoma. In some embodiments, the cancer is follicular lymphoma, diffuse large B-cell lymphoma (DLBCL), mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma. In some embodiments, the cancer is acute or chronic myelogenous (or myeloid) leukemia, myelodysplastic syndrome, or acute lymphoblastic leukemia. In some embodiments, the cancer is relapsed or refractory diffuse large B-cell lymphoma (DLBCL), relapsed or refractory mantle cell lymphoma, relapsed or refractory follicular lymphoma, relapsed or refractory CLL; relapsed or refractory SLL; relapsed or refractory multiple myeloma. In some embodiments, the cancer is high risk CLL or high risk SLL.

In some embodiments, the dose of Ibrutinib is between about 10 mg to about 100 mg. In some embodiments, the therapeutically-effective amount of Ibrutinib is between about 40 mg and about 100 mg. In some embodiments, the dose of Ibrutinib is between about 40 mg and about 70 mg. In some embodiments, the dose of Ibrutinib is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the dose of Ibrutinib is about 40 mg. In some embodiments, the method increases the Cmax of Ibrutinib. In some embodiments, Cmax of Ibrutinib is increased by about 20× to about 40× the Cmax of Ibrutinib administered without a Second anticancer agent, or about 25× to about 35×. In some embodiments, the method increases the AUC of Ibrutinib. In some embodiments, the method increases the AUC of Ibrutinib by about 15× to about 35× the AUC of Ibrutinib administered without a Second anticancer agent, or about 20× to about 30×. In some embodiments, the method increases the AUC of Ibrutinib by about 2× to about 35× the AUC of Ibrutinib administered without a Second anticancer agent. In some embodiments, the method increases the AUC of Ibrutinib by about 2× to about 30× the AUC of Ibrutinib administered without a Second anticancer agent. In some embodiments, the method increases the AUC of Ibrutinib by about 2× to about 25× the AUC of Ibrutinib administered without a Second anticancer agent. In some embodiments, the method increases the AUC of Ibrutinib by about 2× to about 20× the AUC of Ibrutinib administered without a Second anticancer agent. In some embodiments, the method increases the AUC of Ibrutinib by about 2× to about 15× the AUC of Ibrutinib administered without a Second anticancer agent. In some embodiments, the method increases the AUC of Ibrutinib by about 2× to about 10× the AUC of Ibrutinib administered without a Second anticancer agent. In some embodiments, the method increases the AUC of Ibrutinib by about 2× to about 5× the AUC of Ibrutinib administered without a Second anticancer agent. In some embodiments, the method increases the AUC of Ibrutinib by about 2× to about 4× the AUC of Ibrutinib administered without a Second anticancer agent. In some embodiments, the method does not significantly affect the Tmax or T1/2 of Ibrutinib as compared to the Tmax and T1/2 of Ibrutinib administered without a Second anticancer agent. In some embodiments, Ibrutinib and the Second anticancer agent are in a combined dosage form. In some embodiments, Ibrutinib and the Second anticancer agent are in separate dosage forms. In some embodiments, Ibrutinib and the Second anticancer agent are administered concurrently. In some embodiments, Ibrutinib and the Second anticancer agent are administered simultaneously, essentially simultaneously or within the same treatment protocol. In some embodiments, Ibrutinib and the Second anticancer agent are administered sequentially. In some embodiments, Ibrutinib is amorphous or crystalline.

B-cell proliferative disorders (BCPDs) are neoplasms of the blood and encompass, inter alia, non-Hodgkin lymphoma, multiple myeloma, and leukemia. BCPDs can originate either in the lymphatic tissues (as in the case of lymphoma) or in the bone marrow (as in the case of leukemia and myeloma), and they all are involved with the uncontrolled growth of lymphocytes or white blood cells. There are many subtypes of BCPD, e.g., chronic lymphocytic leukemia (CLL) and non-Hodgkin lymphoma (NHL). The disease course and treatment of BCPD is dependent on the BCPD subtype; however, even within each subtype the clinical presentation, morphologic appearance, and response to therapy is heterogeneous.

Malignant lymphomas are neoplastic transformations of cells that reside predominantly within lymphoid tissues. Two groups of malignant lymphomas are Hodgkin's lymphoma and non-Hodgkin's lymphoma (NHL). Both types of lymphomas infiltrate reticuloendothelial tissues. However, they differ in the neoplastic cell of origin, site of disease, presence of systemic symptoms, and response to treatment (Freedman et al., “Non-Hodgkin's Lymphomas” Chapter 134, Cancer Medicine, (an approved publication of the American Cancer Society, B.C. Decker Inc., Hamilton, Ontario, 2003).

### Non-Hodgkin's Lymphomas

Disclosed herein, in certain embodiments, is a method for treating a non-Hodgkin's lymphoma in an individual in need thereof, comprising: administering a combination of a Btk inhibitor and a Second anticancer agent.

Disclosed herein, in certain embodiments, is a method for treating a non-Hodgkin's lymphoma in an individual in need thereof, comprising: administering a combination of Ibrutinib and a Second anticancer agent.

Further disclosed herein, in certain embodiments, is a method for treating relapsed or refractory non-Hodgkin's lymphoma in an individual in need thereof, comprising: administering to the individual a combination of a Btk inhibitor and a Second anticancer agent. In some embodiments, the non-Hodgkin's lymphoma is relapsed or refractory diffuse large B-cell lymphoma (DLBCL), relapsed or refractory mantle cell lymphoma, or relapsed or refractory follicular lymphoma.

Further disclosed herein, in certain embodiments, is a method for treating relapsed or refractory non-Hodgkin's lymphoma in an individual in need thereof, comprising: administering to the individual a combination of Ibrutinib and a Second anticancer agent. In some embodiments, the non-Hodgkin's lymphoma is relapsed or refractory diffuse large B-cell lymphoma (DLBCL), relapsed or refractory mantle cell lymphoma, or relapsed or refractory follicular lymphoma.

Non-Hodgkin lymphomas (NHL) are a diverse group of malignancies that are predominately of B-cell origin. NHL may develop in any organs associated with lymphatic system such as spleen, lymph nodes or tonsils and can occur at any age. NHL is often marked by enlarged lymph nodes, fever, and weight loss. NHL is classified as either B-cell or T-cell NHL. Lymphomas related to lymphoproliferative disorders following bone marrow or stem cell transplantation are usually B-cell NHL. In the Working Formulation classification scheme, NHL has been divided into low-, intermediate-, and high-grade categories by virtue of their natural histories (see “The Non-Hodgkin's Lymphoma Pathologic Classification Project,” Cancer 49(1982):2112-2135). The low-grade lymphomas are indolent, with a median survival of 5 to 10 years (Horning and Rosenberg (1984) N. Engl. J. Med. 311:1471-1475). Although chemotherapy can induce remissions in the majority of indolent lymphomas, cures are rare and most patients eventually relapse, requiring further therapy. The intermediate- and high-grade lymphomas are more aggressive tumors, but they have a greater chance for cure with chemotherapy. However, a significant proportion of these patients will relapse and require further treatment.

A non-limiting list of the B-cell NHL includes Burkitt's lymphoma (e.g., Endemic Burkitt's Lymphoma and Sporadic Burkitt's Lymphoma), Cutaneous B-Cell Lymphoma, Cutaneous Marginal Zone Lymphoma (MZL), Diffuse Large Cell Lymphoma (DLBCL), Diffuse Mixed Small and Large Cell Lymphoma, Diffuse Small Cleaved Cell, Diffuse Small Lymphocytic Lymphoma, Extranodal Marginal Zone B-cell lymphoma, follicular lymphoma, Follicular Small Cleaved Cell (Grade 1), Follicular Mixed Small Cleaved and Large Cell (Grade 2), Follicular Large Cell (Grade 3), Intravascular Large B-Cell Lymphoma, Intravascular Lymphomatosis, Large Cell Immunoblastic Lymphoma, Large Cell Lymphoma (LCL), Lymphoblastic Lymphoma, MALT Lymphoma, Mantle Cell Lymphoma (MCL), immunoblastic large cell lymphoma, precursor B-lymphoblastic lymphoma, mantle cell lymphoma, chronic lymphocytic leukemia (CLL)/small lymphocytic lymphoma (SLL), extranodal marginal zone B-cell lymphoma-mucosa-associated lymphoid tissue (MALT) lymphoma, Mediastinal Large B-Cell Lymphoma, nodal marginal zone B-cell lymphoma, splenic marginal zone B-cell lymphoma, primary mediastinal B-cell lymphoma, lymphoplasmocytic lymphoma, hairy cell leukemia, Waldenstrom's Macroglobulinemia, and primary central nervous system (CNS) lymphoma. Additional non-Hodgkin's lymphomas are contemplated within the scope of the present invention and apparent to those of ordinary skill in the art.

### DLBCL

Disclosed herein, in certain embodiments, is a method for treating a DLCBL in an individual in need thereof, comprising: administering a combination of a Btk inhibitor and a Second anticancer agent.

Further disclosed herein, in certain embodiments, is a method for treating a DLCBL in an individual in need thereof, comprising: administering a combination of Ibrutinib and a Second anticancer agent.

As used herein, the term “Diffuse large B-cell lymphoma (DLBCL)” refers to a neoplasm of the germinal center B lymphocytes with a diffuse growth pattern and a high-intermediate proliferation index. DLBCLs represent approximately 30% of all lymphomas and may present with several morphological variants including the centroblastic, immunoblastic, T-cell/histiocyte rich, anaplastic and plasmoblastic subtypes. Genetic tests have shown that there are different subtypes of DLBCL. These subtypes seem to have different outlooks (prognoses) and responses to treatment. DLBCL can affect any age group but occurs mostly in older people (the average age is mid-60s).

Disclosed herein, in certain embodiments, is a method for treating diffuse large B-cell lymphoma, activated B cell-like subtype (ABC-DLBCL), in an individual in need thereof, comprising: administering to the individual a combination of Ibrutinib and a Second anticancer agent. The ABC subtype of diffuse large B-cell lymphoma (ABC-DLBCL) is thought to arise from post germinal center B cells that are arrested during plasmatic differentiation. The ABC subtype of DLBCL (ABC-DLBCL) accounts for approximately 30% total DLBCL diagnoses. It is considered the least curable of the DLBCL molecular subtypes and, as such, patients diagnosed with the ABC-DLBCL typically display significantly reduced survival rates compared with individuals with other types of DLCBL. ABC-DLBCL is most commonly associated with chromosomal translocations deregulating the germinal center master regulator BCL6 and with mutations inactivating the PRDM1 gene, which encodes a transcriptional repressor required for plasma cell differentiation.

A particularly relevant signaling pathway in the pathogenesis of ABC-DLBCL is the one mediated by the nuclear factor (NF)-κB transcription complex. The NF-κB family comprises 5 members (p50, p52, p65, c-rel and RelB) that form homo- and heterodimers and function as transcriptional factors to mediate a variety of proliferation, apoptosis, inflammatory and immune responses and are critical for normal B-cell development and survival. NF-κB is widely used by eukaryotic cells as a regulator of genes that control cell proliferation and cell survival. As such, many different types of human tumors have misregulated NF-κB: that is, NF-κB is constitutively active. Active NF-κB turns on the expression of genes that keep the cell proliferating and protect the cell from conditions that would otherwise cause it to die via apoptosis.

The dependence of ABC DLBCLs on NF-kB depends on a signaling pathway upstream of IkB kinase comprised of CARD11, BCL10 and MALT1 (the CBM complex). Interference with the CBM pathway extinguishes NF-kB signaling in ABC DLBCL cells and induces apoptosis. The molecular basis for constitutive activity of the NF-kB pathway is a subject of current investigation but some somatic alterations to the genome of ABC DLBCLs clearly invoke this pathway. For example, somatic mutations of the coiled-coil domain of CARD11 in DLBCL render this signaling scaffold protein able to spontaneously nucleate protein-protein interaction with MALT1 and BCL10, causing IKK activity and NF-kB activation. Constitutive activity of the B cell receptor signaling pathway has been implicated in the activation of NF-kB in ABC DLBCLs with wild type CARD11, and this is associated with mutations within the cytoplasmic tails of the B cell receptor subunits CD79A and CD79B. Oncogenic activating mutations in the signaling adapter MYD88 activate NF-kB and synergize with B cell receptor signaling in sustaining the survival of ABC DLBCL cells. In addition, inactivating mutations in a negative regulator of the NF-kB pathway, A20, occur almost exclusively in ABC DLBCL.

Indeed, genetic alterations affecting multiple components of the NF-κB signaling pathway have been recently identified in more than 50% of ABC-DLBCL patients, where these lesions promote constitutive NF-κB activation, thereby contributing to lymphoma growth. These include mutations of CARD11 (˜10% of the cases), a lymphocyte-specific cytoplasmic scaffolding protein that—together with MALT1 and BCL10—forms the BCR signalosome, which relays signals from antigen receptors to the downstream mediators of NF-κB activation. An even larger fraction of cases (˜30%) carry biallelic genetic lesions inactivating the negative NF-κB regulator A20. Further, high levels of expression of NF-κB target genes have been observed in ABC-DLBCL tumor samples. See, e.g., U. Klein et al., (2008), Nature Reviews Immunology 8:22-23; R. E. Davis et al., (2001), Journal of Experimental Medicine 194:1861-1874; G. Lentz et al., (2008), Science 319:1676-1679; M. Compagno et al., (2009), Nature 459:712-721; and L. Srinivasan et al., (2009), Cell 139:573-586).

DLBCL cells of the ABC subtype, such as OCI-Ly10, have chronic active BCR signaling and are very sensitive to the Btk inhibitor described herein. The irreversible Btk inhibitor described herein potently and irreversibly inhibits the growth of OCI-Ly10 (EC50 continuous exposure=10 nM, EC50 1 hour pulse=50 nM). In addition, induction of apoptosis, as shown by capsase activation, Annexin-V flow cytometry and increase in sub-GO fraction is observed in OCILy10. Both sensitive and resistant cells express Btk at similar levels, and the active site of Btk is fully occupied by the inhibitor in both as shown using a fluorescently labeled affinity probe. OCI-Ly10 cells are shown to have chronically active BCR signaling to NF-kB which is dose dependently inhibited by the Btk inhibitors described herein. The activity of Btk inhibitors in the cell lines studied herein are also characterized by comparing signal transduction profiles (Btk, PLCγ, ERK, NF-kB, AKT), cytokine secretion profiles and mRNA expression profiles, both with and without BCR stimulation, and observed significant differences in these profiles that lead to clinical biomarkers that identify the most sensitive patient populations to Btk inhibitor treatment. See U.S. Pat. No. 7,711,492 and Staudt et al., Nature, Vol. 463, Jan. 7, 2010, pp. 88-92, the contents of which are incorporated by reference in their entirety.

### Follicular Lymphoma

Disclosed herein, in certain embodiments, is a method for treating a follicular lymphoma in an individual in need thereof, comprising: administering a combination of a Btk inhibitor and a Second anticancer agent.

Further disclosed herein, in certain embodiments, is a method for treating a follicular lymphoma in an individual in need thereof, comprising: administering a combination of Ibrutinib and a Second anticancer agent.

As used herein, the term “follicular lymphoma” refers to any of several types of non-Hodgkin's lymphoma in which the lymphomatous cells are clustered into nodules or follicles. The term follicular is used because the cells tend to grow in a circular, or nodular, pattern in lymph nodes. The average age for people with this lymphoma is about 60.

### CLL/SLL

Disclosed herein, in certain embodiments, is a method for treating a CLL or SLL in an individual in need thereof, comprising: administering a combination of a Btk inhibitor and a Second anticancer agent.

Further disclosed herein, in certain embodiments, is a method for treating a CLL or SLL in an individual in need thereof, comprising: administering a combination of Ibrutinib and a Second anticancer agent.

Chronic lymphocytic leukemia and small lymphocytic lymphoma (CLL/SLL) are commonly thought as the same disease with slightly different manifestations. Where the cancerous cells gather determines whether it is called CLL or SLL. When the cancer cells are primarily found in the lymph nodes, lima bean shaped structures of the lymphatic system (a system primarily of tiny vessels found in the body), it is called SLL. SLL accounts for about 5% to 10% of all lymphomas. When most of the cancer cells are in the bloodstream and the bone marrow, it is called CLL.

Both CLL and SLL are slow-growing diseases, although CLL, which is much more common, tends to grow slower. CLL and SLL are treated the same way. They are usually not considered curable with standard treatments, but depending on the stage and growth rate of the disease, most patients live longer than 10 years. Occasionally over time, these slow-growing lymphomas may transform into a more aggressive type of lymphoma.

Chronic lymphoid leukemia (CLL) is the most common type of leukemia. It is estimated that 100,760 people in the United States are living with or are in remission from CLL. Most (>75%) people newly diagnosed with CLL are over the age of 50. Currently CLL treatment focuses on controlling the disease and its symptoms rather than on an outright cure. CLL is treated by chemotherapy, radiation therapy, biological therapy, or bone marrow transplantation. Symptoms are sometimes treated surgically (splenectomy removal of enlarged spleen) or by radiation therapy (“de-bulking” swollen lymph nodes). Though CLL progresses slowly in most cases, it is considered generally incurable. Certain CLLs are classified as high-risk. As used herein, “high risk CLL” means CLL characterized by at least one of the following 1) 17p13-; 2) 11q22-; 3) unmutated IgVH together with ZAP-70+ and/or CD38+; or 4) trisomy 12.

CLL treatment is typically administered when the patient's clinical symptoms or blood counts indicate that the disease has progressed to a point where it may affect the patient's quality of life.

Small lymphocytic leukemia (SLL) is very similar to CLL described supra, and is also a cancer of B-cells. In SLL the abnormal lymphocytes mainly affect the lymph nodes. However, in CLL the abnormal cells mainly affect the blood and the bone marrow. The spleen may be affected in both conditions. SLL accounts for about 1 in 25 of all cases of non-Hodgkin lymphoma. It can occur at any time from young adulthood to old age, but is rare under the age of 50. SLL is considered an indolent lymphoma. This means that the disease progresses very slowly, and patients tend to live many years after diagnosis. However, most patients are diagnosed with advanced disease, and although SLL responds well to a variety of chemotherapy drugs, it is generally considered to be incurable. Although some cancers tend to occur more often in one gender or the other, cases and deaths due to SLL are evenly split between men and women. The average age at the time of diagnosis is 60 years.

Although SLL is indolent, it is persistently progressive. The usual pattern of this disease is one of high response rates to radiation therapy and/or chemotherapy, with a period of disease remission. This is followed months or years later by an inevitable relapse. Re-treatment leads to a response again, but again the disease will relapse. This means that although the short-term prognosis of SLL is quite good, over time, many patients develop fatal complications of recurrent disease. Considering the age of the individuals typically diagnosed with CLL and SLL, there is a need in the art for a simple and effective treatment of the disease with minimum side-effects that do not impede on the patient's quality of life. The instant invention fulfills this long standing need in the art.

### Mantle Cell Lymphoma

Disclosed herein, in certain embodiments, is a method for treating a Mantle cell lymphoma in an individual in need thereof, comprising: administering a combination of a Btk inhibitor and a Second anticancer agent.

Further disclosed herein, in certain embodiments, is a method for treating a Mantle cell lymphoma in an individual in need thereof, comprising: administering a combination of Ibrutinib and a Second anticancer agent.

As used herein, the term, “Mantle cell lymphoma” refers to a subtype of B-cell lymphoma, due to CD5 positive antigen-naive pregerminal center B-cell within the mantle zone that surrounds normal germinal center follicles. MCL cells generally over-express cyclin Dl due to a t(11:14) chromosomal translocation in the DNA. More specifically, the translocation is at t(11;14)(q13;q32). Only about 5% of lymphomas are of this type. The cells are small to medium in size. Men are affected most often. The average age of patients is in the early 60s. The lymphoma is usually widespread when it is diagnosed, involving lymph nodes, bone marrow, and, very often, the spleen. Mantle cell lymphoma is not a very fast growing lymphoma, but is difficult to treat.

### Marginal Zone B-Cell Lymphoma

Disclosed herein, in certain embodiments, is a method for treating a marginal zone B-cell lymphoma in an individual in need thereof, comprising: administering a combination of a Btk inhibitor and a Second anticancer agent.

Further disclosed herein, in certain embodiments, is a method for treating a marginal zone B-cell lymphoma in an individual in need thereof, comprising: administering a combination of Ibrutinib and a Second anticancer agent.

As used herein, the term “marginal zone B-cell lymphoma” refers to a group of related B-cell neoplasms that involve the lymphoid tissues in the marginal zone, the patchy area outside the follicular mantle zone. Marginal zone lymphomas account for about 5% to 10% of lymphomas. The cells in these lymphomas look small under the microscope. There are 3 main types of marginal zone lymphomas including extranodal marginal zone B-cell lymphomas, nodal marginal zone B-cell lymphoma, and splenic marginal zone lymphoma.

### MALT

Disclosed herein, in certain embodiments, is a method for treating a MALT in an individual in need thereof, comprising: administering a combination of a Btk inhibitor and a Second anticancer agent.

Further disclosed herein, in certain embodiments, is a method for treating a MALT in an individual in need thereof, comprising: administering a combination of Ibrutinib and a Second anticancer agent.

The term “mucosa-associated lymphoid tissue (MALT) lymphoma”, as used herein, refers to extranodal manifestations of marginal-zone lymphomas. Most MALT lymphoma are a low grade, although a minority either manifest initially as intermediate-grade non-Hodgkin lymphoma (NHL) or evolve from the low-grade form. Most of the MALT lymphoma occur in the stomach, and roughly 70% of gastric MALT lymphoma are associated with Helicobacter pylori infection. Several cytogenetic abnormalities have been identified, the most common being trisomy 3 or t(11;18). Many of these other MALT lymphoma have also been linked to infections with bacteria or viruses. The average age of patients with MALT lymphoma is about 60.

### Nodal Marginal Zone B-Cell Lymphoma

Disclosed herein, in certain embodiments, is a method for treating a nodal marginal zone B-cell lymphoma in an individual in need thereof, comprising: administering a combination of a Btk inhibitor and a Second anticancer agent.

Further disclosed herein, in certain embodiments, is a method for treating a nodal marginal zone B-cell lymphoma in an individual in need thereof, comprising: administering a combination of Ibrutinib and a Second anticancer agent.

The term “nodal marginal zone B-cell lymphoma” refers to an indolent B-cell lymphoma that is found mostly in the lymph nodes. The disease is rare and only accounts for 1% of all Non-Hodgkin's Lymphomas (NHL). It is most commonly diagnosed in older patients, with women more susceptible than men. The disease is classified as a marginal zone lymphoma because the mutation occurs in the marginal zone of the B-cells. Due to its confinement in the lymph nodes, this disease is also classified as nodal.

### Splenic Marginal Zone B-Cell Lymphoma

Disclosed herein, in certain embodiments, is a method for treating a splenic marginal zone B-cell lymphoma in an individual in need thereof, comprising: administering a combination of a Btk inhibitor and a Second anticancer agent.

Further disclosed herein, in certain embodiments, is a method for treating a splenic marginal zone B-cell lymphoma in an individual in need thereof, comprising: administering a combination of Ibrutinib and a Second anticancer agent.

The term “splenic marginal zone B-cell lymphoma” refers to specific low-grade small B-cell lymphoma that is incorporated in the World Health Organization classification. Characteristic features are splenomegaly, moderate lymphocytosis with villous morphology, intrasinusoidal pattern of involvement of various organs, especially bone marrow, and relative indolent course. Tumor progression with increase of blastic forms and aggressive behavior are observed in a minority of patients. Molecular and cytogenetic studies have shown heterogeneous results probably because of the lack of standardized diagnostic criteria.

### Burkitt Lymphoma

Disclosed herein, in certain embodiments, is a method for treating a Burkitt lymphoma in an individual in need thereof, comprising: administering a combination of a Btk inhibitor and a Second anticancer agent.

Further disclosed herein, in certain embodiments, is a method for treating a Burkitt lymphoma in an individual in need thereof, comprising: administering a combination of Ibrutinib and a Second anticancer agent.

The term “Burkitt lymphoma” refers to a type of Non-Hodgkin Lymphoma (NHL) that commonly affects children. It is a highly aggressive type of B-cell lymphoma that often starts and involves body parts other than lymph nodes. In spite of its fast-growing nature, Burkitt's lymphoma is often curable with modern intensive therapies. There are two broad types of Burkitt's lymphoma—the sporadic and the endemic varieties:

Endemic Burkitt's lymphoma: The disease involves children much more than adults, and is related to Epstein Barr Virus (EBV) infection in 95% cases. It occurs primarily is equatorial Africa, where about half of all childhood cancers are Burkitt's lymphoma. It characteristically has a high chance of involving the jawbone, a rather distinctive feature that is rare in sporadic Burkitt's. It also commonly involves the abdomen.

Sporadic Burkitt's lymphoma: The type of Burkitt's lymphoma that affects the rest of the world, including Europe and the Americas is the sporadic type. Here too, it's mainly a disease in children. The link between Epstein Barr Virus (EBV) is not as strong as with the endemic variety, though direct evidence of EBV infection is present in one out of five patients. More than the involvement of lymph nodes, it is the abdomen that is notably affected in more than 90% of the children. Bone marrow involvement is more common than in the sporadic variety.

### Waldenstrom Macroglobulinemia

Disclosed herein, in certain embodiments, is a method for treating a Waldenstrom macroglobulinemia in an individual in need thereof, comprising: administering a combination of a Btk inhibitor and a Second anticancer agent.

Further disclosed herein, in certain embodiments, is a method for treating a Waldenstrom macroglobulinemia in an individual in need thereof, comprising: administering a combination of Ibrutinib and a Second anticancer agent.

The term “Waldenstrom macroglobulinemia”, also known as lymphoplasmacytic lymphoma, is cancer involving a subtype of white blood cells called lymphocytes. It is characterized by an uncontrolled clonal proliferation of terminally differentiated B lymphocytes. It is also characterized by the lymphoma cells making an antibody called immunoglobulin M (IgM). The IgM antibodies circulate in the blood in large amounts, and cause the liquid part of the blood to thicken, like syrup. This can lead to decreased blood flow to many organs, which can cause problems with vision (because of poor circulation in blood vessels in the back of the eyes) and neurological problems (such as headache, dizziness, and confusion) caused by poor blood flow within the brain. Other symptoms can include feeling tired and weak, and a tendency to bleed easily. The underlying etiology is not fully understood but a number of risk factors have been identified, including the locus 6p21.3 on chromosome 6. There is a 2- to 3-fold risk increase of developing WM in people with a personal history of autoimmune diseases with autoantibodies and particularly elevated risks associated with hepatitis, human immunodeficiency virus, and rickettsiosis.

### Multiple Myeloma

Disclosed herein, in certain embodiments, is a method for treating a myeloma in an individual in need thereof, comprising: administering a combination of a Btk inhibitor and a Second anticancer agent.

Further disclosed herein, in certain embodiments, is a method for treating a myeloma in an individual in need thereof, comprising: administering a combination of Ibrutinib and a Second anticancer agent.

Multiple myeloma, also known as MM, myeloma, plasma cell myeloma, or as Kahler's disease (after Otto Kahler) is a cancer of the white blood cells known as plasma cells. A type of B cell, plasma cells are a crucial part of the immune system responsible for the production of antibodies in humans and other vertebrates. They are produced in the bone marrow and are transported through the lymphatic system.

### Leukemia

Disclosed herein, in certain embodiments, is a method for treating a leukemia in an individual in need thereof, comprising: administering a combination of a Btk inhibitor and a Second anticancer agent.

Further disclosed herein, in certain embodiments, is a method for treating a leukemia in an individual in need thereof, comprising: administering a combination of Ibrutinib and a Second anticancer agent.

Leukemia is a cancer of the blood or bone marrow characterized by an abnormal increase of blood cells, usually leukocytes (white blood cells). Leukemia is a broad term covering a spectrum of diseases. The first division is between its acute and chronic forms: (i) acute leukemia is characterized by the rapid increase of immature blood cells. This crowding makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children; (ii) chronic leukemia is distinguished by the excessive build up of relatively mature, but still abnormal, white blood cells. Typically taking months or years to progress, the cells are produced at a much higher rate than normal cells, resulting in many abnormal white blood cells in the blood. Chronic leukemia mostly occurs in older people, but can theoretically occur in any age group. Additionally, the diseases are subdivided according to which kind of blood cell is affected. This split divides leukemias into lymphoblastic or lymphocytic leukemias and myeloid or myelogenous leukemias: (i) lymphoblastic or lymphocytic leukemias, the cancerous change takes place in a type of marrow cell that normally goes on to form lymphocytes, which are infection-fighting immune system cells; (ii) myeloid or myelogenous leukemias, the cancerous change takes place in a type of marrow cell that normally goes on to form red blood cells, some other types of white cells, and platelets.

Within these main categories, there are several subcategories including, but not limited to, Acute lymphoblastic leukemia (ALL), Acute myelogenous leukemia (AML), Chronic myelogenous leukemia (CML), and Hairy cell leukemia (HCL).

Symptoms, diagnostic tests, and prognostic tests for each of the above-mentioned conditions are known. See, e.g., Harrison's Principles of Internal Medicine©,” 16th ed., 2004, The McGraw-Hill Companies, Inc. Dey et al. (2006), Cytojournal 3(24), and the “Revised European American Lymphoma” (REAL) classification system (see, e.g., the website maintained by the National Cancer Institute).

A number of animal models are useful for establishing a range of therapeutically effective doses of irreversible Btk inhibitor compounds, such as Ibrutinib, for treating any of the foregoing diseases.

The therapeutic efficacy of Ibrutinib for any one of the foregoing diseases can be optimized during a course of treatment. For example, a subject being treated can undergo a diagnostic evaluation to correlate the relief of disease symptoms or pathologies to inhibition of in vivo Btk activity achieved by administering a given dose of Ibrutinib. Cellular assays known in the art can be used to determine in vivo activity of Btk in the presence or absence of an irreversible Btk inhibitor. For example, since activated Btk is phosphorylated at tyrosine 223 (Y223) and tyrosine 551 (Y551), phospho-specific immunocytochemical staining of P-Y223 or P-Y551-positive cells can be used to detect or quantify activation of Btk in a population of cells (e.g., by FACS analysis of stained vs unstained cells). See, e.g., Nisitani et al. (1999), Proc. Natl. Acad. Sci, USA 96:2221-2226. Thus, the amount of the Btk inhibitor compound that is administered to a subject can be increased or decreased as needed so as to maintain a level of Btk inhibition optimal for treating the subject's disease state.

Ibrutinib can irreversibly inhibit Btk and may be used to treat mammals suffering from Bruton's tyrosine kinase-dependent or Bruton's tyrosine kinase mediated conditions or diseases, including, but not limited to, cancer, autoimmune and other inflammatory diseases. Ibrutinib has shown efficacy is a wide variety of diseases and conditions that are described herein.

In some embodiments, a Btk inhibitor and a Second anticancer agent are used for the manufacture of a medicament for treating any of the foregoing conditions (e.g., autoimmune diseases, inflammatory diseases, allergy disorders, B-cell proliferative disorders, or thromboembolic disorders).

In some embodiments, Ibrutinib and a Second anticancer agent are used for the manufacture of a medicament for treating any of the foregoing conditions (e.g., autoimmune diseases, inflammatory diseases, allergy disorders, B-cell proliferative disorders, or thromboembolic disorders).

### Additional Combination Therapies

In certain instances, it is appropriate to administer a Btk inhibitor and a Second anticancer agent in combination with an additional therapeutic agent. In certain instances, it is appropriate to administer Ibrutinib and a Second anticancer agent in combination with an additional therapeutic agent. Additional therapeutic agents are selected for their particular usefulness against the condition that is being treated. In general, the additional therapeutic agent does not need to be administered in the same pharmaceutical composition, at the same time or via the same route and the Ibrutinib and/or Second anticancer agent. In one embodiment, the initial administration is made according to established protocols, and then, based upon the observed effects, the dosage, modes of administration and times of administration, further modified.

In some embodiments, the additional therapeutic agent is administered concurrently (e.g., simultaneously, essentially simultaneously or within the same treatment protocol) or sequentially, depending upon the nature of the disease, the condition of the patient, and the actual choice of compounds used. In certain embodiments, the determination of the order of administration, and the number of repetitions of administration of each therapeutic agent during a treatment protocol, is based upon evaluation of the disease being treated and the condition of the patient.

The dose of the additional therapeutic agent varies depending on the additional therapeutic agent, the disease or condition being treated and so forth.

### Pharmaceutical Compositions/Formulations

Disclosed herein, in certain embodiments, are pharmaceutical compositions comprising (a) a Btk inhibitor and a second anticancer agent. Further disclosed herein, in certain embodiments, are pharmaceutical compositions comprising (a) Ibrutinib and a second anticancer agent, and (b) a pharmaceutically-acceptable excipient.

In some embodiments, the second anticancer agent inhibits Bcl-2; Janus kinase 2 (JAK2); Anaplastic lymphoma kinase (ALK); or heat shock protein 90 (Hsp90), wherein the combination provides a synergistic therapeutic effect compared to administration of ibrutinib or the second anticancer agent alone. In some embodiments, the second anticancer agent inhibits Bcl-2. In some embodiments, the second anticancer agent that inhibits Bcl-2 is selected from ABT-737, ABT-199 and HA14-1. In some embodiments, the second anticancer agent inhibits JAK2. In some embodiments, the second anticancer agent that inhibits JAK2 is TG-101348. In some embodiments, the second anticancer agent inhibits ALK. In some embodiments, the second anticancer agent that inhibits ALK is NVP-TAE684. In some embodiments, the second anticancer agent inhibits Hsp90. In some embodiments, the second anticancer agent that inhibits Hsp 90 is 17-DMAG.

In some embodiments, the second anticancer agent is a glucocorticoid, a vinca alkaloid, an anti-metabolite, a DNA damaging agent, lenalidomide, rituximab, or a PKC perturbagen, wherein the combination provides a synergistic therapeutic effect compared to administration of ibrutinib or the second anticancer agent alone. In some embodiments, the second anticancer agent is a glucocorticoid. In some embodiments, the second anticancer agent is selected from dexamethasone and prednisolone. In some embodiments, the second anticancer agent is a vinca alkaloid. In some embodiments, the second anticancer agent is vincristine. In some embodiments, the second anticancer agent is an anti-metabolite. In some embodiments, the second anticancer agent is gemcitabine. In some embodiments, the second anticancer agent is a DNA damaging agent. In some embodiments, the DNA damaging agent is selected from carboplatin and chlorambucil. In some embodiments, the second anticancer agent is lenalidomide. In some embodiments, the second anticancer agent is rituximab. In some embodiments, the second anticancer agent is a PKC perturbagen. In some embodiments, the PKC perturbagen is selected from enzastarin and GF109203X.

In some embodiments, the second anticancer agent inhibits a B-cell receptor pathway kinase selected from among Lyn/Fyn, Syk, PI3K, PKCβ, and IKK, wherein the combination provides a synergistic therapeutic effect compared to administration of ibrutinib or the second anticancer agent alone. In some embodiments, the second anticancer agent inhibits a B-cell receptor pathway kinase selected from among Lyn/Fyn, Syk, PI3K, PKCβ, and IKK. In some embodiments, the second anticancer agent inhibits Lyn/Fyn. In some embodiments, the second anticancer agent inhibits Syk. In some embodiments, the second anticancer agent is R406. In some embodiments, the second anticancer agent inhibits PKCβ. In some embodiments, the second anticancer agent inhibits IKK. In some embodiments, the second anticancer agent inhibits PI3K. In some embodiments, the second anticancer agent that inhibits PI3K is selected from IPI-145, BKM120, BEZ235, GDC-0941, AMG319, CAL-101 and A66.

In some embodiments, the second anticancer agent inhibits the 20s proteasome, IRF-4, IRAK4, EZH2, CXCR4, CXCR5, GLS, cyclin dependent kinase 4/6 (CDK4/6), topoisomerase II, PLK; DNA methyltransferase, the Ras/MAPK pathway, or FGFR1 tyrosine kinase, wherein the combination provides a synergistic therapeutic effect compared to administration of ibrutinib or the second anticancer agent alone. In some embodiments, the second anticancer agent inhibits the 20s proteasome. In some embodiments, the second anticancer agent is carfilzomib. In some embodiments, the second anticancer agent inhibits IRF-4. In some embodiments, the second anticancer agent is LEN. In some embodiments, the second anticancer agent inhibits IRAK4. In some embodiments, the second anticancer agent is ND-2158. In some embodiments, the second anticancer agent inhibits EZH2. In some embodiments, the second anticancer agent is selected from EI1, GSK343 and EPZ005687. In some embodiments, the second anticancer agent inhibits CXCR4. In some embodiments, the second anticancer agent is AMD3100. In some embodiments, the second anticancer agent inhibits CXCR5. In some embodiments, the second anticancer agent is an antibody against CXCR5. In some embodiments, wherein the second anticancer agent inhibits GLS. In some embodiments, the second anticancer agent is JNJ-16. In some embodiments, wherein the second anticancer agent inhibits CDK4/6. In some embodiments, the second anticancer agent is JNJ-08. In some embodiments, the second anticancer agent inhibits topoisomerase II. In some embodiments, the second anticancer agent is selected from doxorubicin and etoposide. In some embodiments, the second anticancer agent inhibits PLK. In some embodiments, the second anticancer agent is selected from BI-2536 and GSK461364. In some embodiments, the second anticancer agent inhibits DNA methyltransferase. In some embodiments, the second anticancer agent is azacitidine. In some embodiments, the second anticancer agent inhibits the Ras/MAPK pathway. In some embodiments, the second anticancer agent is selected from sorafenib and PLX-4032. In some embodiments, the second anticancer agent inhibits FGFR1 tyrosine kinase. In some embodiments, the second anticancer agent is JNJ-13.

In some embodiments, the second anticancer agent is selected from AZD0503, dasatinib and nilotinib, and JNJ-20, wherein the combination provides a synergistic therapeutic effect compared to administration of ibrutinib or the second anticancer agent alone. In some embodiments, the second anticancer agent is AZD0503. In some embodiments, the second anticancer agent is dasatinib. In some embodiments, the second anticancer agent is nilotinib. In some embodiments, the second anticancer agent is JNJ-20.

In some embodiments, the dose of Ibrutinib is between about 10 mg to about 100 mg. In some embodiments, the therapeutically-effective amount of Ibrutinib is between about 40 mg and about 100 mg. In some embodiments, the dose of Ibrutinib is between about 40 mg and about 70 mg. In some embodiments, the dose of Ibrutinib is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the dose of Ibrutinib is about 40 mg. In some embodiments, Ibrutinib is amorphous or crystalline. In some embodiments, Ibrutinib is milled or a nano-particle. In some embodiments, the pharmaceutical composition is a combined dosage form. In some embodiments, the composition increases the oral bioavailability of Ibrutinib. In some embodiments, the composition increases the Cmax of Ibrutinib. In some embodiments, the composition increases the AUC of Ibrutinib. In some embodiments, the composition increases the Cmax of Ibrutinib by about 20× to about 40× the Cmax of Ibrutinib administered without a Second anticancer agent, or about 25× to about 35×. In some embodiments, the composition increases the AUC of Ibrutinib by about 15× to about 35× the AUC of Ibrutinib administered without a Second anticancer agent, or about 20× to about 30×. In some embodiments, the composition comprises an amount of the Second anticancer agent that is effective to increase the AUC of Ibrutinib by about 2× to about 35× the AUC of Ibrutinib administered without a Second anticancer agent. In some embodiments, the composition comprises an amount of the Second anticancer agent that is effective to increase the AUC of Ibrutinib by about 2× to about 30× the AUC of Ibrutinib administered without a Second anticancer agent. In some embodiments, the composition comprises an amount of the Second anticancer agent that is effective to increase the AUC of Ibrutinib by about 2× to about 25× the AUC of Ibrutinib administered without a Second anticancer agent. In some embodiments, the composition comprises an amount of the Second anticancer agent that is effective to increase the AUC of Ibrutinib by about 2× to about 20× the AUC of Ibrutinib administered without a Second anticancer agent. In some embodiments, the composition comprises an amount of the Second anticancer agent that is effective to increase the AUC of Ibrutinib by about 2× to about 15× the AUC of Ibrutinib administered without a Second anticancer agent. In some embodiments, the composition comprises an amount of the Second anticancer agent that is effective to increase the AUC of Ibrutinib by about 2× to about 10× the AUC of Ibrutinib administered without a Second anticancer agent. In some embodiments, the composition comprises an amount of the Second anticancer agent that is effective to increase the AUC of Ibrutinib by about 2× to about 5× the AUC of Ibrutinib administered without a Second anticancer agent. In some embodiments, the composition comprises an amount of the Second anticancer agent that is effective to increase the AUC of Ibrutinib by about 2× to about 4× the AUC of Ibrutinib administered without a Second anticancer agent. In some embodiments, the composition does not significantly affect the Tmax or T1/2 of Ibrutinib as compared to the Tmax and T1/2 of Ibrutinib administered without a Second anticancer agent. In some embodiments, the pharmaceutical compositions further comprise chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, the pharmaceutical compositions further comprise cyclophosphamide, hydroxydaunorubicin, vincristine, and prednisone, and optionally, rituximab. In some embodiments, the pharmaceutical compositions further comprise bendamustine, and rituximab. In some embodiments, the pharmaceutical compositions further comprise fludarabine, cyclophosphamide, and rituximab. In some embodiments, the pharmaceutical compositions further comprise cyclophosphamide, vincristine, and prednisone, and optionally, rituximab. In some embodiments, the pharmaceutical compositions further comprise etoposide, doxorubicin, vincristine, cyclophosphamide, prednisolone, and optionally, rituximab. In some embodiments, the pharmaceutical compositions further comprise dexamethasone and lenalidomide.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference in their entirety.

A pharmaceutical composition, as used herein, refers to a mixture of Ibrutinib, a Second anticancer agent, and/or an additional therapeutic agent with other chemical components, such as carriers, stabilizers, diluents, dispersing agents, suspending agents, thickening agents, and/or excipients.

In practicing the methods of treatment or use provided herein, therapeutically effective amounts of the compounds disclosed herein are administered having a disease, disorder, or condition to be treated. In some embodiments, the mammal is a human. The therapeutically effective amounts of the compounds may vary depending on the compounds, severity of the disease, the age and relative health of the subject, and other factors.

The term “combination” as used herein, means a product that results from the mixing or combining of Ibrutinib and a Second anticancer agent (and any additional therapeutic agents) and includes both fixed and non-fixed combinations. The term “fixed combination” means that Ibrutinib and the Second anticancer agent are both administered in a single entity or dosage form. The term “non-fixed combination” means that Ibrutinib and the Second anticancer agent are administered as separate entities or dosage forms either simultaneously, concurrently or sequentially with no specific intervening time limits, wherein such administration provides effective levels of the two compounds in the body of the patient. The latter also applies to cocktail therapy, e.g. the administration of three or more active ingredients.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

### Dosage Forms

Disclosed herein, in certain embodiments, are dosage forms comprising a Btk inhibitor and a Second anticancer agent. Further disclosed herein, in certain embodiments, are dosage forms comprising Ibrutinib and a Second anticancer agent. In some embodiments, the dosage form is a combined dosage form. In some embodiments, the dosage form is a solid oral dosage form. In some embodiments, the dosage form is a tablet, pill, or capsule. In some embodiments, the dosage form is a controlled release dosage form, delayed release dosage form, extended release dosage form, pulsatile release dosage form, multiparticulate dosage form, or mixed immediate release and controlled release formulation. In some embodiments, the dosage form comprises a controlled release coating. In some embodiments, the dosage forms comprises a first controlled release coating which controls the release of Ibrutinib and a second controlled release coating which controls the release of the Second anticancer agent.

In some embodiments, the second anticancer agent inhibits Bcl-2; Janus kinase 2 (JAK2); Anaplastic lymphoma kinase (ALK); or heat shock protein 90 (Hsp90), wherein the combination provides a synergistic therapeutic effect compared to administration of ibrutinib or the second anticancer agent alone. In some embodiments, the second anticancer agent inhibits Bcl-2. In some embodiments, the second anticancer agent that inhibits Bcl-2 is selected from ABT-737, ABT-199 and HA14-1. In some embodiments, the second anticancer agent inhibits JAK2. In some embodiments, the second anticancer agent that inhibits JAK2 is TG-101348. In some embodiments, the second anticancer agent inhibits ALK. In some embodiments, the second anticancer agent that inhibits ALK is NVP-TAE684. In some embodiments, the second anticancer agent inhibits Hsp90. In some embodiments, the second anticancer agent that inhibits Hsp 90 is 17-DMAG.

In some embodiments, the second anticancer agent is a glucocorticoid, a vinca alkaloid, an anti-metabolite, a DNA damaging agent, lenalidomide, rituximab, or a PKC perturbagen, wherein the combination provides a synergistic therapeutic effect compared to administration of ibrutinib or the second anticancer agent alone. In some embodiments, the second anticancer agent is a glucocorticoid. In some embodiments, the second anticancer agent is selected from dexamethasone and prednisolone. In some embodiments, the second anticancer agent is a vinca alkaloid. In some embodiments, the second anticancer agent is vincristine. In some embodiments, the second anticancer agent is an anti-metabolite. In some embodiments, the second anticancer agent is gemcitabine. In some embodiments, the second anticancer agent is a DNA damaging agent. In some embodiments, the DNA damaging agent is selected from carboplatin and chlorambucil. In some embodiments, the second anticancer agent is lenalidomide. In some embodiments, the second anticancer agent is rituximab. In some embodiments, the second anticancer agent is a PKC perturbagen. In some embodiments, the PKC perturbagen is selected from enzastarin and GF109203X.

In some embodiments, the second anticancer agent inhibits a B-cell receptor pathway kinase selected from among Lyn/Fyn, Syk, PI3K, PKCβ, and IKK, wherein the combination provides a synergistic therapeutic effect compared to administration of ibrutinib or the second anticancer agent alone. In some embodiments, the second anticancer agent inhibits a B-cell receptor pathway kinase selected from among Lyn/Fyn, Syk, PI3K, PKCβ, and IKK. In some embodiments, the second anticancer agent inhibits Lyn/Fyn. In some embodiments, the second anticancer agent inhibits Syk. In some embodiments, the second anticancer agent is R406. In some embodiments, the second anticancer agent inhibits PKCβ. In some embodiments, the second anticancer agent inhibits IKK. In some embodiments, the second anticancer agent inhibits PI3K. In some embodiments, the second anticancer agent that inhibits PI3K is selected from IPI-145, BKM120, BEZ235, GDC-0941, AMG319, CAL-101 and A66.

In some embodiments, the second anticancer agent inhibits the 20s proteasome, IRF-4, IRAK4, EZH2, CXCR4, CXCR5, GLS, cyclin dependent kinase 4/6 (CDK4/6), topoisomerase II, PLK; DNA methyltransferase, the Ras/MAPK pathway, or FGFR1 tyrosine kinase, wherein the combination provides a synergistic therapeutic effect compared to administration of ibrutinib or the second anticancer agent alone. In some embodiments, the second anticancer agent inhibits the 20s proteasome. In some embodiments, the second anticancer agent is carfilzomib. In some embodiments, the second anticancer agent inhibits IRF-4. In some embodiments, the second anticancer agent is LEN. In some embodiments, the second anticancer agent inhibits IRAK4. In some embodiments, the second anticancer agent is ND-2158. In some embodiments, the second anticancer agent inhibits EZH2. In some embodiments, the second anticancer agent is selected from EI1, GSK343 and EPZ005687. In some embodiments, the second anticancer agent inhibits CXCR4. In some embodiments, the second anticancer agent is AMD3100. In some embodiments, the second anticancer agent inhibits CXCR5. In some embodiments, the second anticancer agent is an antibody against CXCR5. In some embodiments, wherein the second anticancer agent inhibits GLS. In some embodiments, the second anticancer agent is JNJ-16. In some embodiments, wherein the second anticancer agent inhibits CDK4/6. In some embodiments, the second anticancer agent is JNJ-08. In some embodiments, the second anticancer agent inhibits topoisomerase II. In some embodiments, the second anticancer agent is selected from doxorubicin and etoposide. In some embodiments, the second anticancer agent inhibits PLK. In some embodiments, the second anticancer agent is selected from BI-2536 and GSK461364. In some embodiments, the second anticancer agent inhibits DNA methyltransferase. In some embodiments, the second anticancer agent is azacitidine. In some embodiments, the second anticancer agent inhibits the Ras/MAPK pathway. In some embodiments, the second anticancer agent is selected from sorafenib and PLX-4032. In some embodiments, the second anticancer agent inhibits FGFR1 tyrosine kinase. In some embodiments, the second anticancer agent is JNJ-13.

In some embodiments, the second anticancer agent is selected from AZD0503, dasatinib and nilotinib, and JNJ-20, wherein the combination provides a synergistic therapeutic effect compared to administration of ibrutinib or the second anticancer agent alone. In some embodiments, the second anticancer agent is AZD0503. In some embodiments, the second anticancer agent is dasatinib. In some embodiments, the second anticancer agent is nilotinib. In some embodiments, the second anticancer agent is JNJ-20.

In some embodiments, the dose of Ibrutinib is between about 5 mg to about 840 mg. In another embodiment, the dose of Ibrutinib is between about 10 mg to about 100 mg. In some embodiments, the therapeutically-effective amount of Ibrutinib is between about 40 mg and about 100 mg. In some embodiments, the dose of Ibrutinib is between about 40 mg and about 70 mg. In some embodiments, the dose of Ibrutinib is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the dose of Ibrutinib is about 40 mg. In other embodiments, the dose of Ibrutinib is about 280 mg. In another embodiment, the dose of Ibrutinib is about 420 mg. In yet another embodiment, the dose of Ibrutinib is about 560 mg. In yet another embodiment, the dose of Ibrutinib is about 700 mg. In yet a further embodiment, the dose of Ibrutinib is about 840 mg. In some embodiments, Ibrutinib is amorphous or crystalline. In some embodiments, the dosage form increases the oral bioavailability of Ibrutinib. In some embodiments, the dosage form increases the Cmax of Ibrutinib. In some embodiments, the dosage form increases the AUC of Ibrutinib. In some embodiments, the dosage form increases the Cmax of Ibrutinib by about 20× to about 40× the Cmax of Ibrutinib administered without a Second anticancer agent, or about 25× to about 35×. In some embodiments, the dosage form increases the AUC of Ibrutinib by about 15× to about 35× the AUC of Ibrutinib administered without a Second anticancer agent, or about 20× to about 30×. In some embodiments, the dosage form increases the AUC of Ibrutinib by about 2× to about 35× the AUC of Ibrutinib administered without a Second anticancer agent. In some embodiments, the dosage form increases the AUC of Ibrutinib by about 2× to about 30× the AUC of Ibrutinib administered without a Second anticancer agent. In some embodiments, the dosage form increases the AUC of Ibrutinib by about 2× to about 25× the AUC of Ibrutinib administered without a Second anticancer agent. In some embodiments, the dosage form increases the AUC of Ibrutinib by about 2× to about 20× the AUC of Ibrutinib administered without a Second anticancer agent. In some embodiments, the dosage form increases the AUC of Ibrutinib by about 2× to about 15× the AUC of Ibrutinib administered without a Second anticancer agent. In some embodiments, the dosage form increases the AUC of Ibrutinib by about 2× to about 10× the AUC of Ibrutinib administered without a Second anticancer agent. In some embodiments, the dosage form increases the AUC of Ibrutinib by about 2× to about 5× the AUC of Ibrutinib administered without a Second anticancer agent. In some embodiments, the dosage form increases the AUC of Ibrutinib by about 2× to about 4× the AUC of Ibrutinib administered without a Second anticancer agent. In some embodiments, the dosage form does not significantly affect the Tmax or T1/2 of Ibrutinib as compared to the Tmax and T1/2 of Ibrutinib administered without a Second anticancer agent. In some embodiments, the dosage forms further comprise chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, the dosage forms further comprise cyclophosphamide, hydroxydaunorubicin, vincristine, and prednisone, and optionally, rituximab. In some embodiments, the dosage forms further comprise bendamustine, and rituximab. In some embodiments, the dosage forms further comprise fludarabine, cyclophosphamide, and rituximab. In some embodiments, the dosage forms further comprise cyclophosphamide, vincristine, and prednisone, and optionally, rituximab. In some embodiments, the dosage forms further comprise etoposide, doxorubicin, vincristine, cyclophosphamide, prednisolone, and optionally, rituximab. In some embodiments, the dosage forms further comprise dexamethasone and lenalidomide.

The pharmaceutical compositions described herein may be formulated for administration via any conventional means including, but not limited to, oral, parenteral (e.g., intravenous, subcutaneous, or intramuscular), buccal, intranasal, rectal or transdermal administration routes. As used herein, the terms “subject”, “individual” and “patient” are used interchangeably and mean an animal, preferably a mammal, including a human or non-human. None of the terms require the supervision (continuous or otherwise) of a medical professional.

The pharmaceutical compositions described herein are formulated into any suitable dosage form, including but not limited to, solid oral dosage forms, controlled release formulations, fast melt formulations, effervescent formulations, tablets, powders, pills, capsules, delayed release formulations, extended release formulations, pulsatile release formulations, multiparticulate formulations, and mixed immediate release and controlled release formulations.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical dosage forms described herein may include one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the pharmaceutical compositions.

### Dosing and Treatment Regimens

In some embodiments, the amount of Ibrutinib that is administered in combination with a Second anticancer agent is from 40 mg/day up to, and including, 1000 mg/day. In some embodiments, the amount of Ibrutinib that is administered is from about 40 mg/day to 70 mg/day. In some embodiments, the amount of Ibrutinib that is administered per day is about 10, mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the amount of Ibrutinib that is administered is about 40 mg/day. In some embodiments, the amount of Ibrutinib that is administered is about 50 mg/day. In some embodiments, the amount of Ibrutinib that is administered is about 60 mg/day. In some embodiments, the amount of Ibrutinib that is administered is about 70 mg/day.

In some embodiments, the AUC0-24 of Ibrutinib co-administered with a Second anticancer agent is between about 50 and about 10000 ng*h/mL. In some embodiments, the Cmax of Ibrutinib co-administered with a Second anticancer agent is between about 5 ng/mL and about 1000 ng/mL.

In some embodiments, Ibrutinib is administered once per day, twice per day, or three times per day. In some embodiments, Ibrutinib is administered once per day. In some embodiments, the Second anticancer agent is administered once per day, twice per day, or three times per day. In some embodiments, the Second anticancer agent is administered once per day. In some embodiments, Ibrutinib and the Second anticancer agent are co-administered (e.g., in a single dosage form), once per day. In some embodiments, Ibrutinib and the Second anticancer agent are maintenance therapy.

In some embodiments, the compositions disclosed herein are administered for prophylactic, therapeutic, or maintenance treatment. In some embodiments, the compositions disclosed herein are administered for therapeutic applications. In some embodiments, the compositions disclosed herein are administered for therapeutic applications. In some embodiments, the compositions disclosed herein are administered as a maintenance therapy, for example for a patient in remission.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, the severity of the disease, the identity (e.g., weight) of the subject or host in need of treatment, but can nevertheless be routinely determined in a manner known in the art according to the particular circumstances surrounding the case, including, e.g., the specific agent being administered, the route of administration, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. Compounds exhibiting high therapeutic indices are preferred. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized.

In some embodiments, the Btk inhibitor and the Second anticancer agent are administered concurrently. In some embodiments, the Btk inhibitor and the Second anticancer agent are administered simultaneously, essentially simultaneously or within the same treatment protocol. In some embodiments, the Btk inhibitor and the Second anticancer agent are administered sequentially.

In some embodiments, Ibrutinib and the Second anticancer agent are administered concurrently. In some embodiments, Ibrutinib and the Second anticancer agent are administered simultaneously, essentially simultaneously or within the same treatment protocol. In some embodiments, Ibrutinib and the Second anticancer agent are administered sequentially.

### Kits/Articles of Manufacture

For use in the therapeutic methods of use described herein, kits and articles of manufacture are also described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include Ibrutinib, optionally in a composition or in combination with a Second anticancer agent as disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

The following ingredients, formulations, processes and procedures for practicing the methods disclosed herein correspond to that described above.

### Example 1

### In Vitro Assay of BTK Inhibitor Combinations in DLBCL Cells

Combinations of the BTK inhibitor ibrutinib and additional anti-cancer cancer agents were assayed using various DLBCL cell lines (TMD8 WT, TMD8 ibrutinib resistant, Ly3, Ly10, DHL2, U2932, HBL1, DHL4, DHL5, SUDHL2, DB, or RCK8 cells). The BTK inhibitor was incubated with other cancer drugs for 2 days. Cell inhibition was assessed by Alamar blue assay.

The combinations tested were:

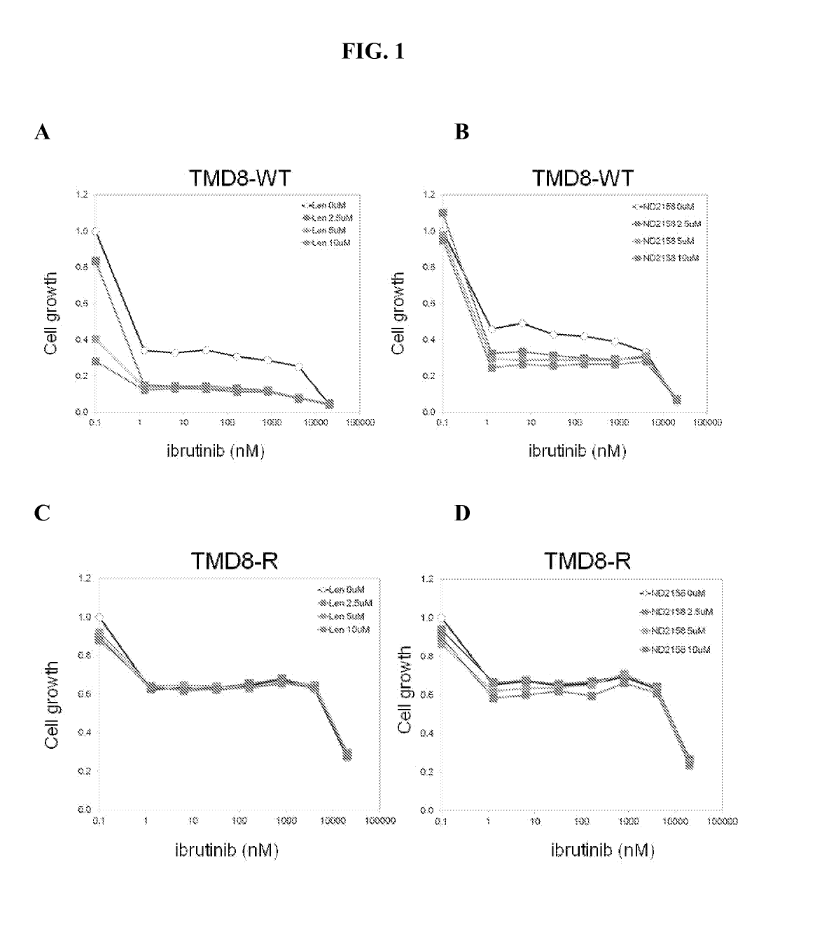

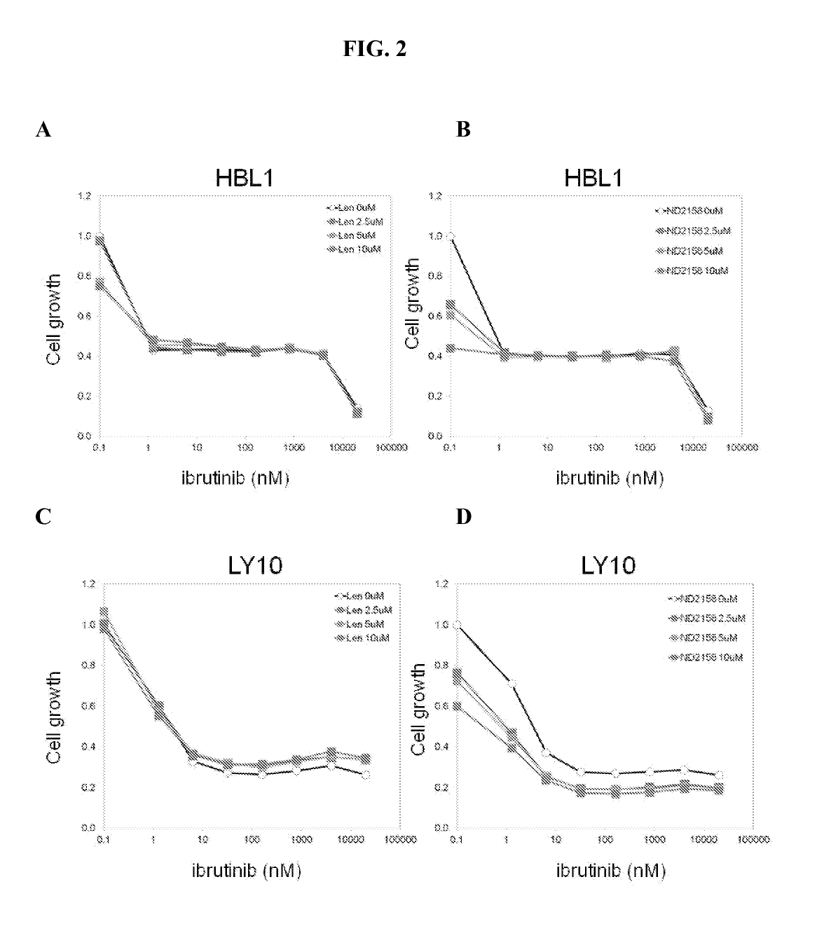

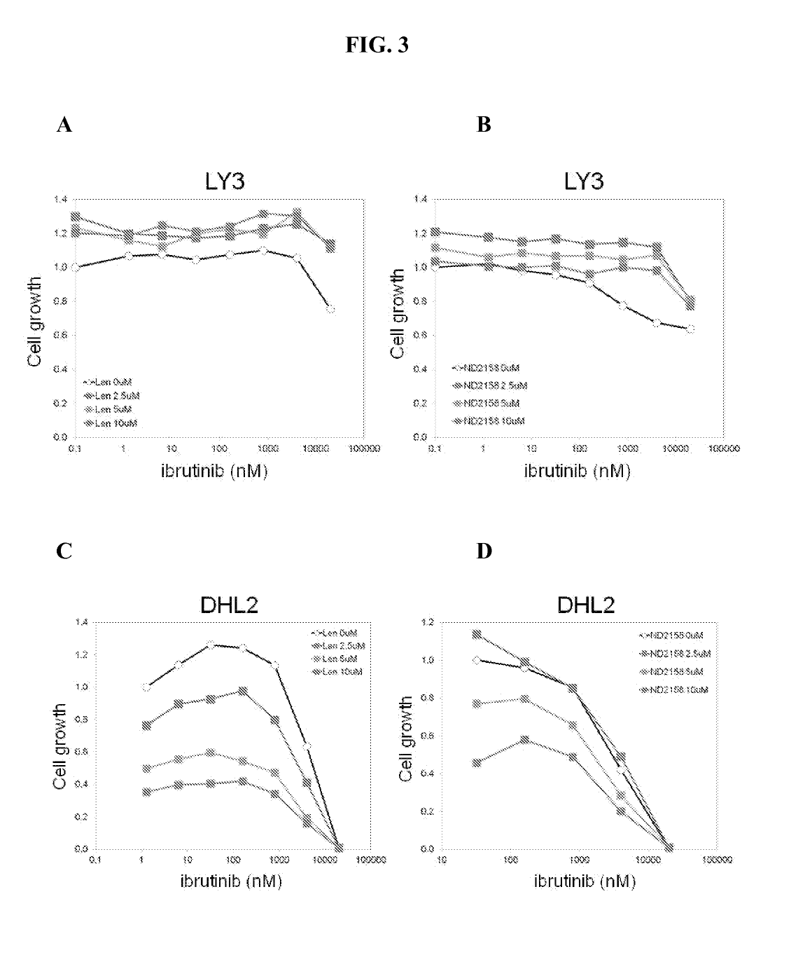

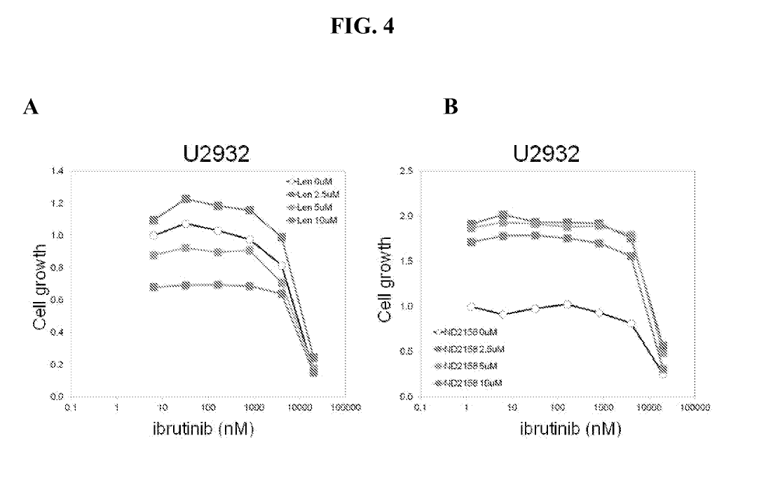

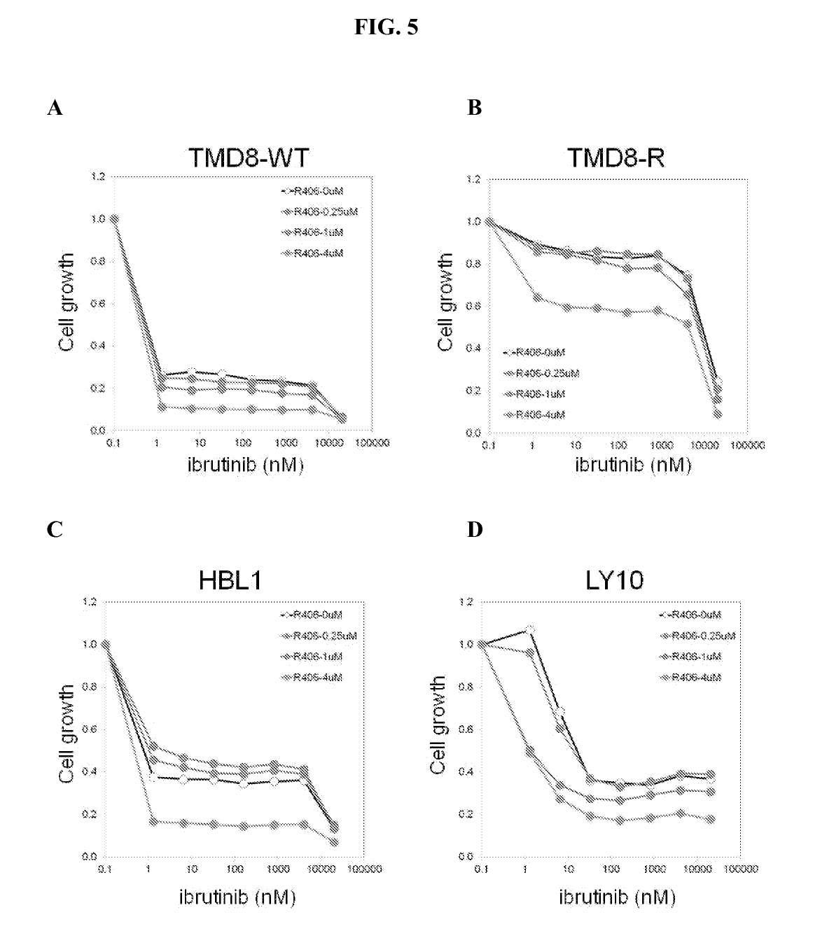

1. Ibrutinib with the IRF-4 inhibitor Lenalidomide (Len) (FIGS. 1A, 1C, 2A, 3A, and 4A).

2. Ibrutinib with the IRAK4 inhibitor ND2158 (FIGS. 1B, 1E, 2B, 3B, and 4B).

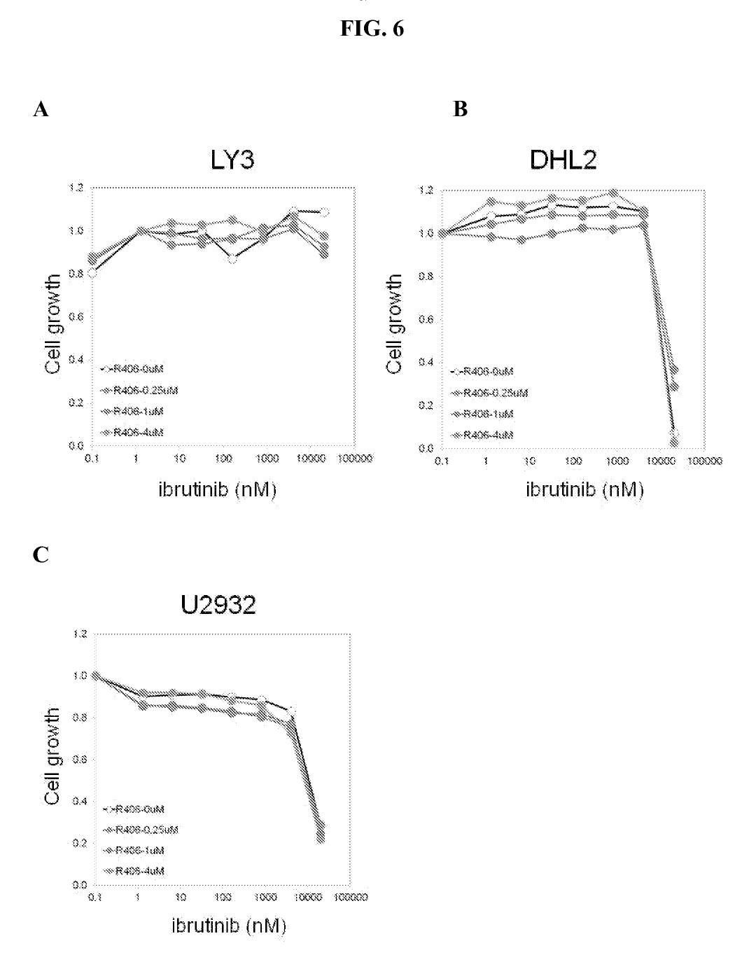

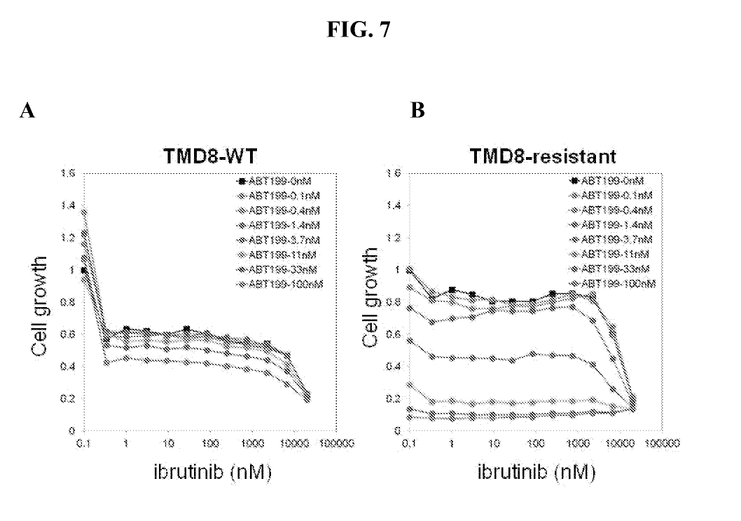

3. Ibrutinib with the SYK inhibitor R406 (FIGS. 5 and 6).

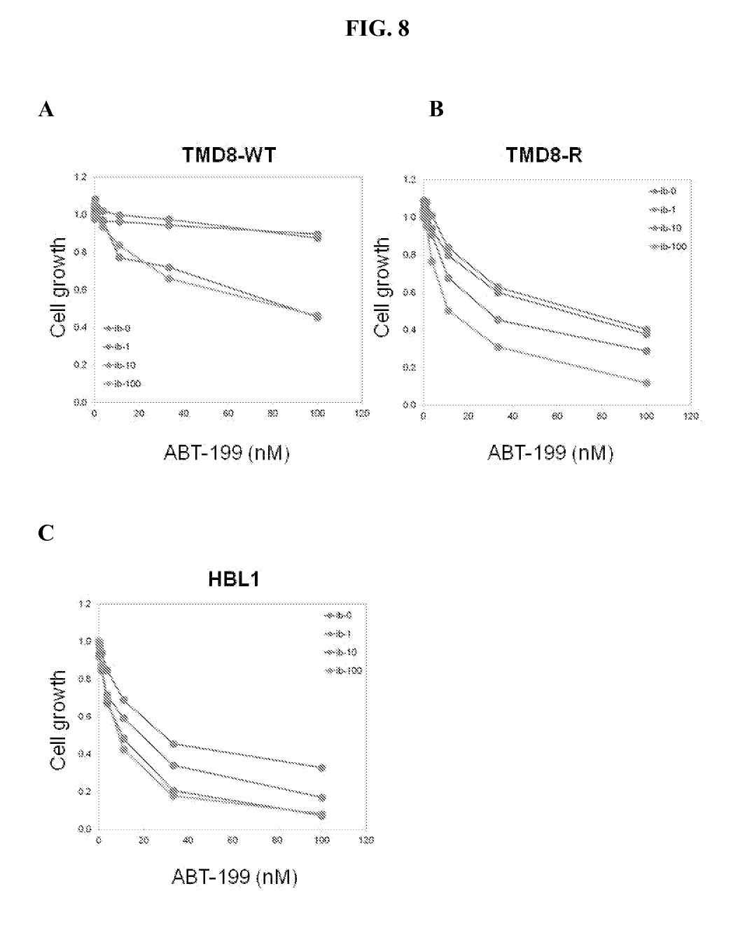

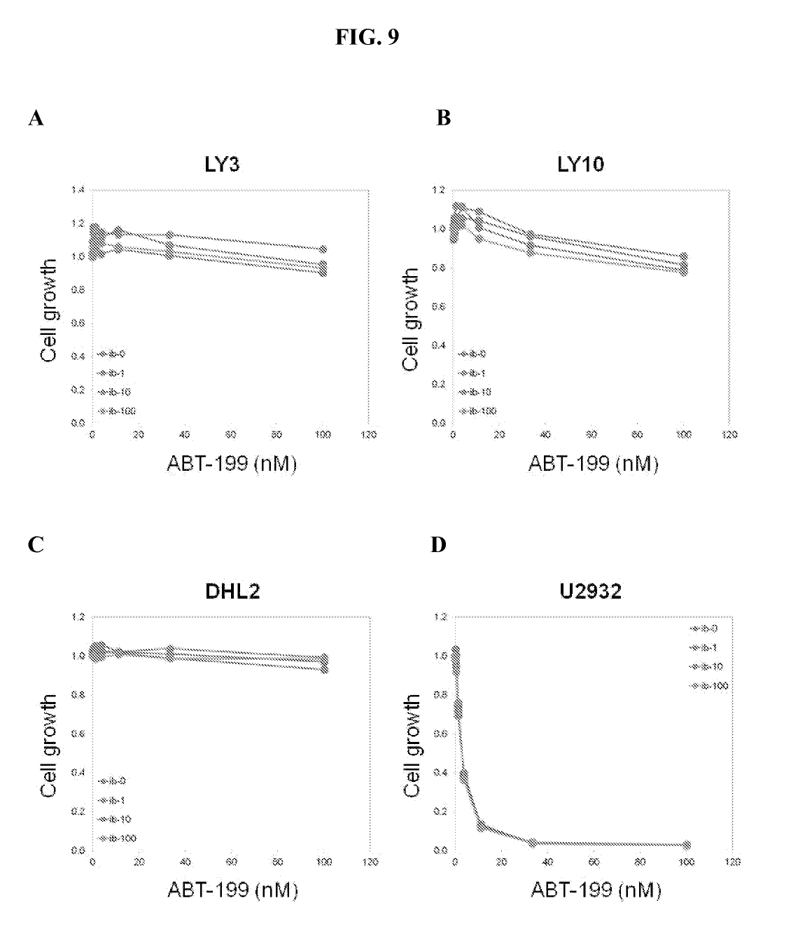

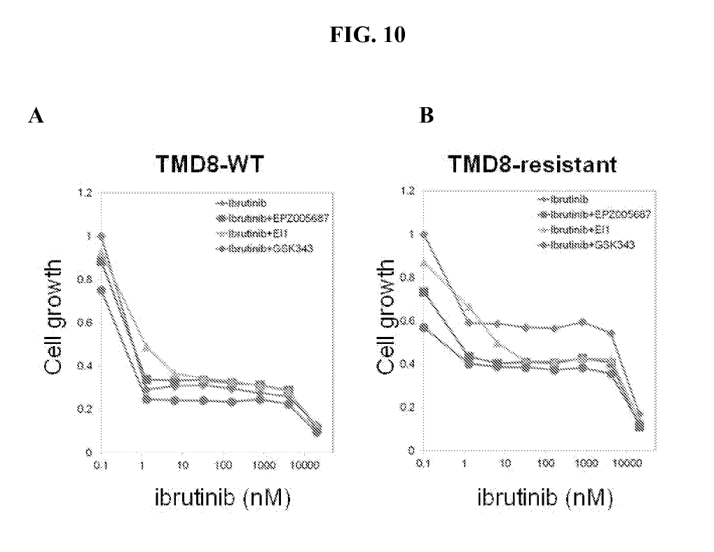

4. Ibrutinib with the BCL-2 inhibitor ABT-199 (FIGS. 7, 8, and 9).

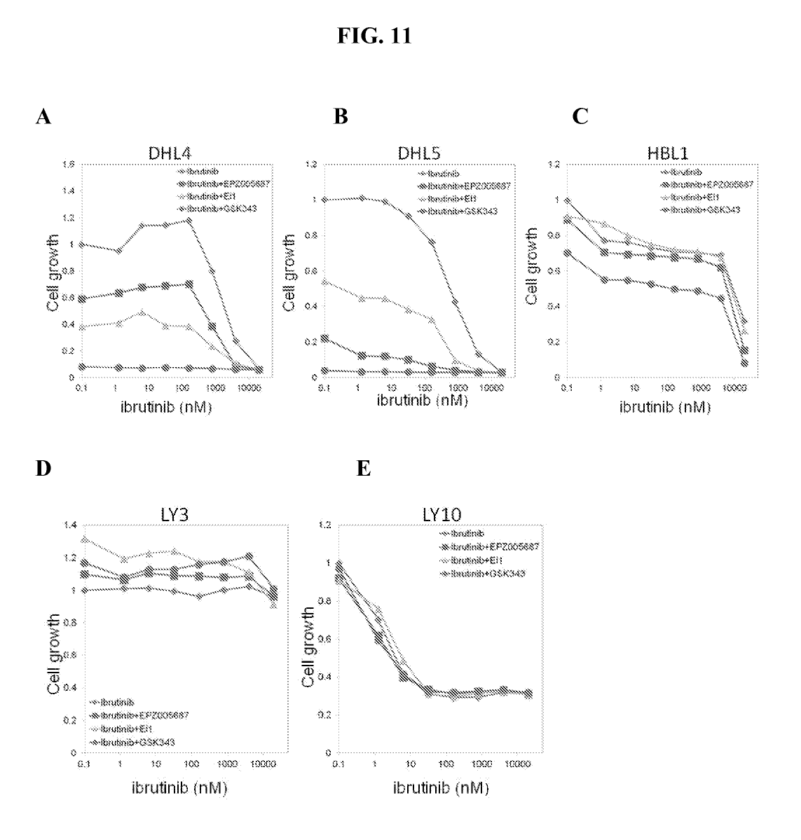

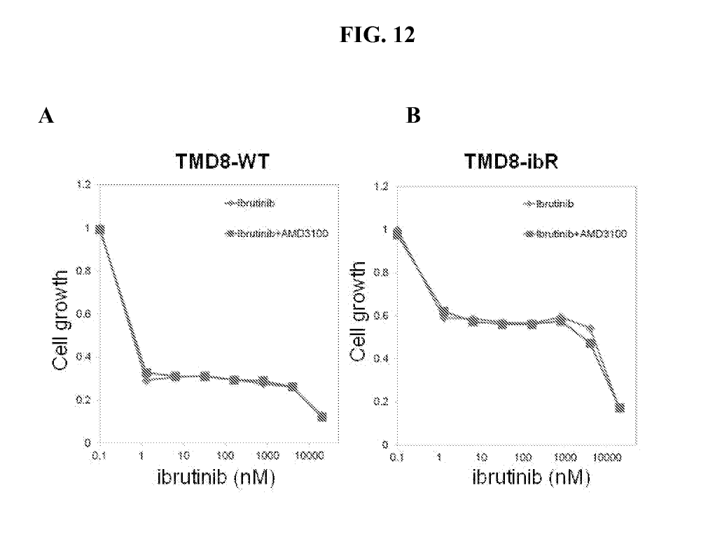

5. Ibrutinib with EZH2 inhibitors EI1, GSK343, or EPZ005687 (FIGS. 10, 11, 12).

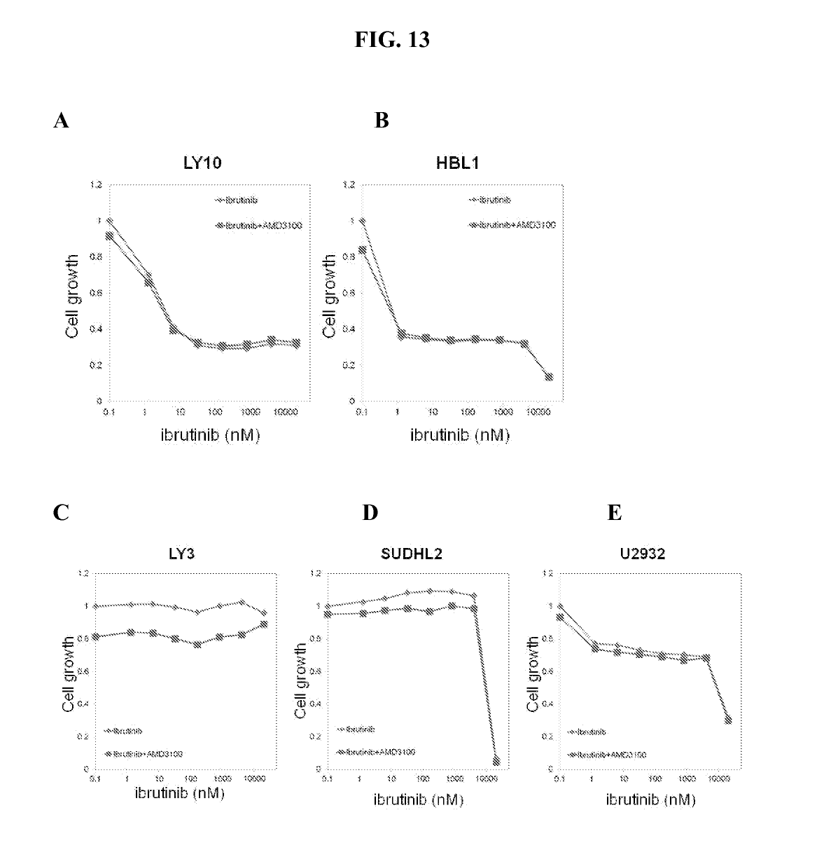

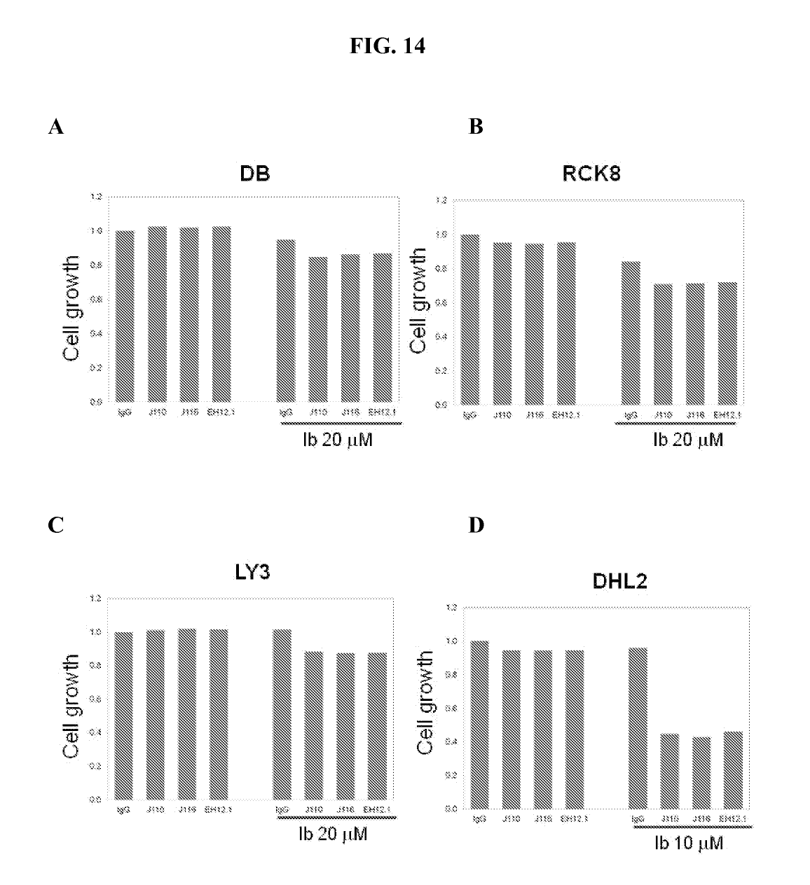

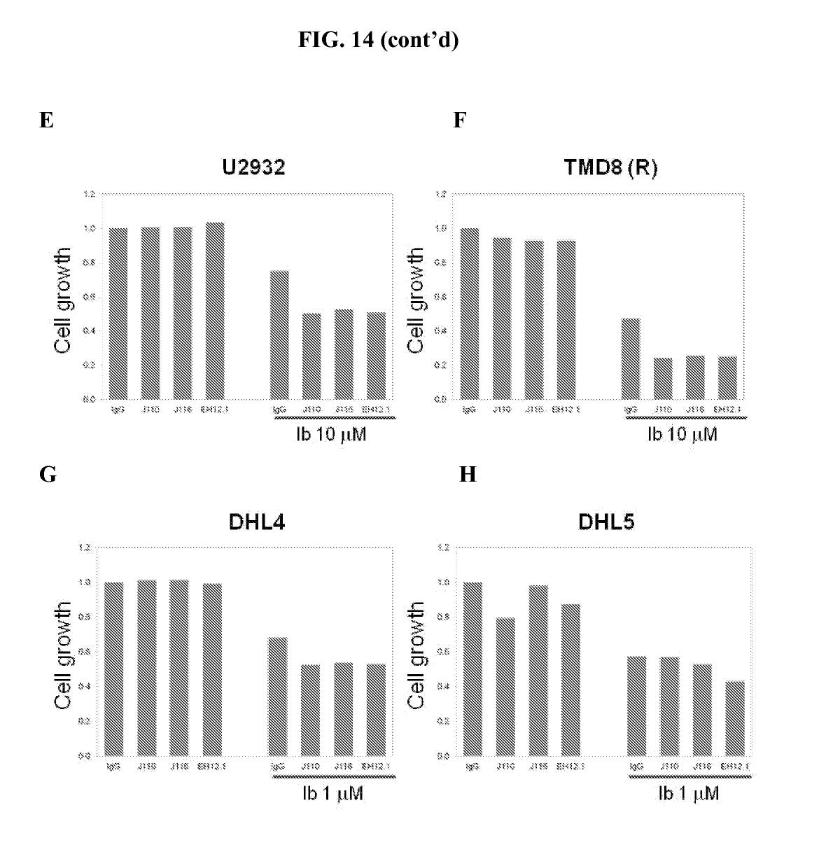

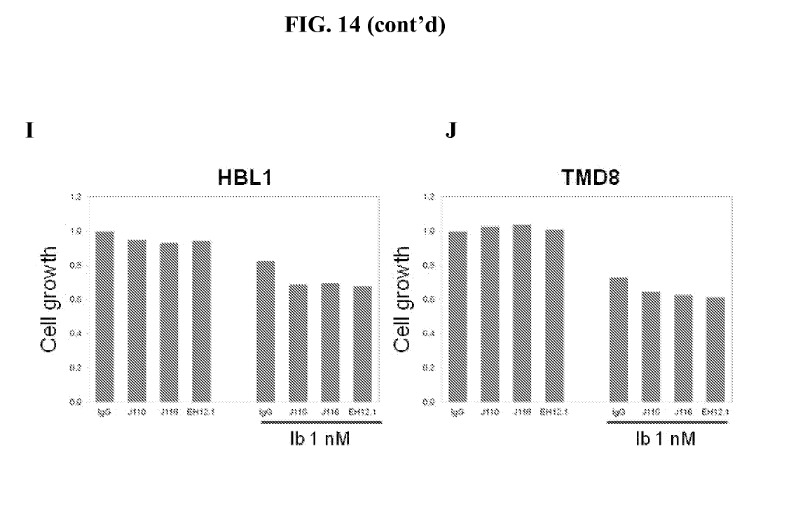

6. Ibrutinib with the CXCR4 inhibitor AMD3100 (FIGS. 13 and 14).

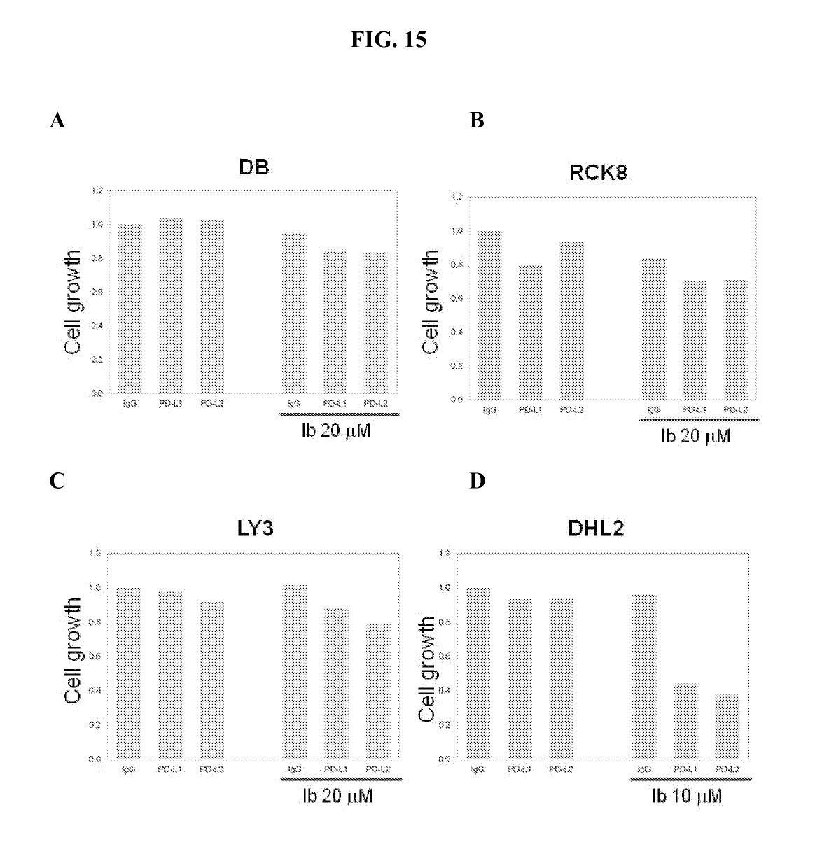

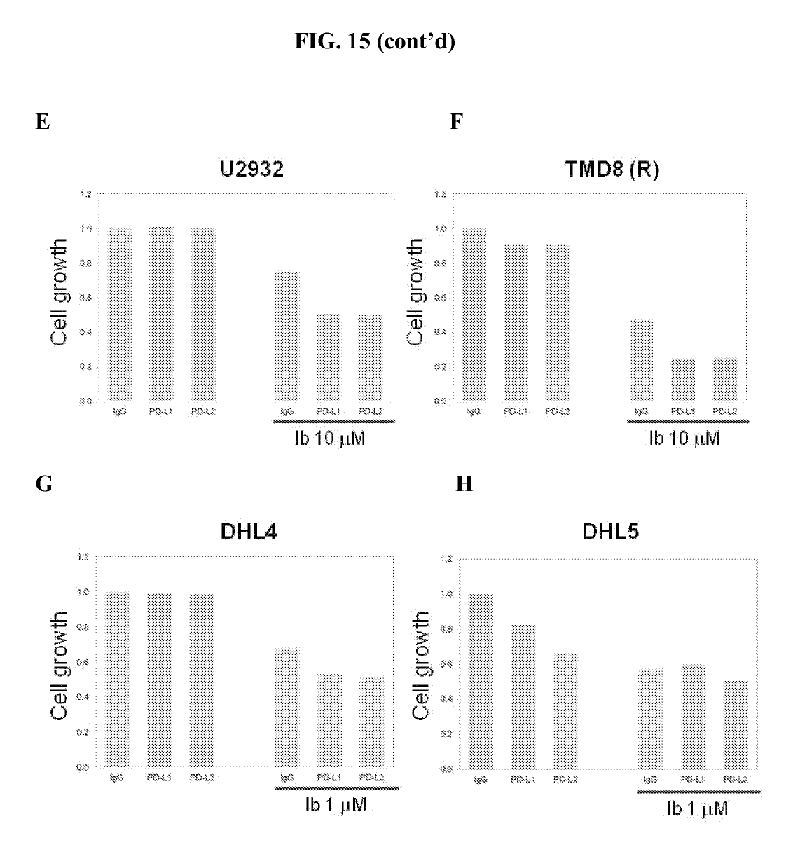

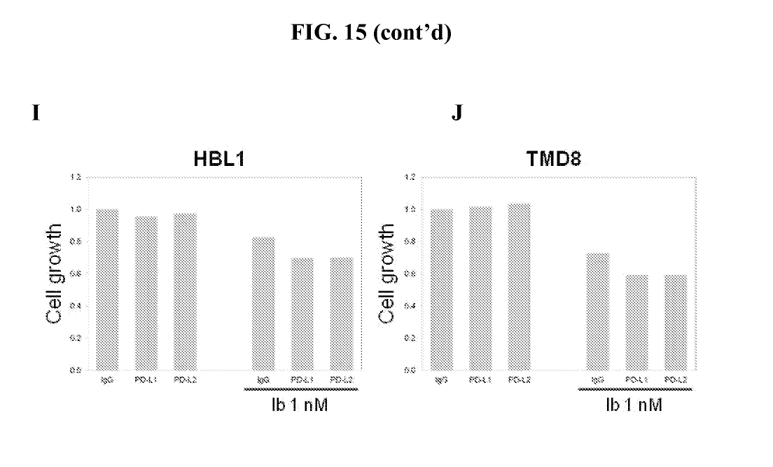

7. Ibrutinib with the PD-1 antibodies J110, J-116, or EH12.1 (FIG. 15).

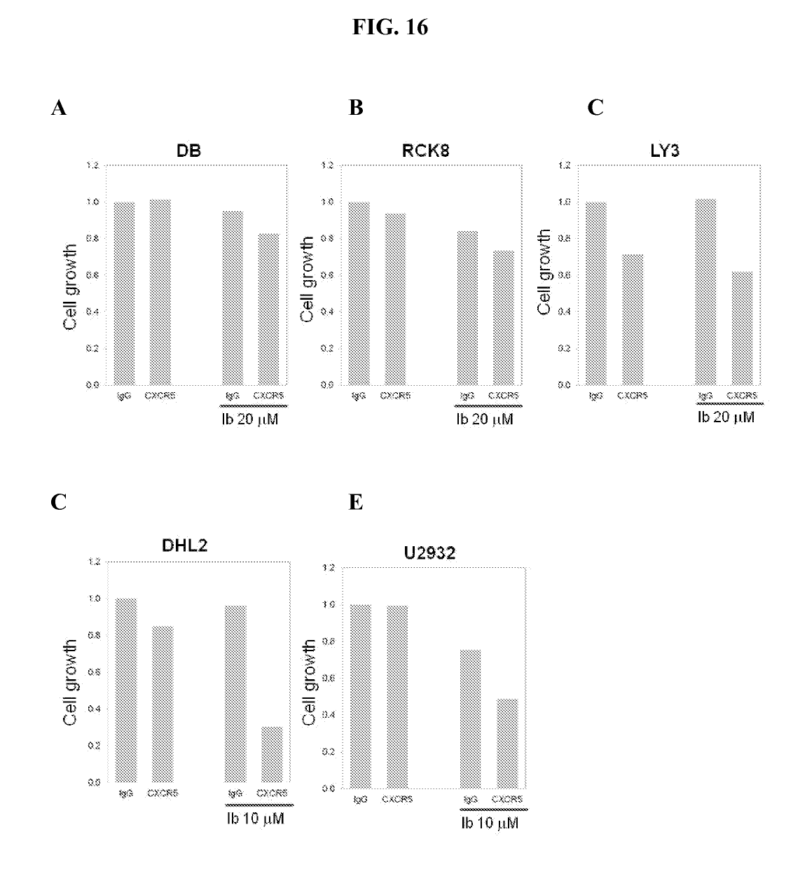

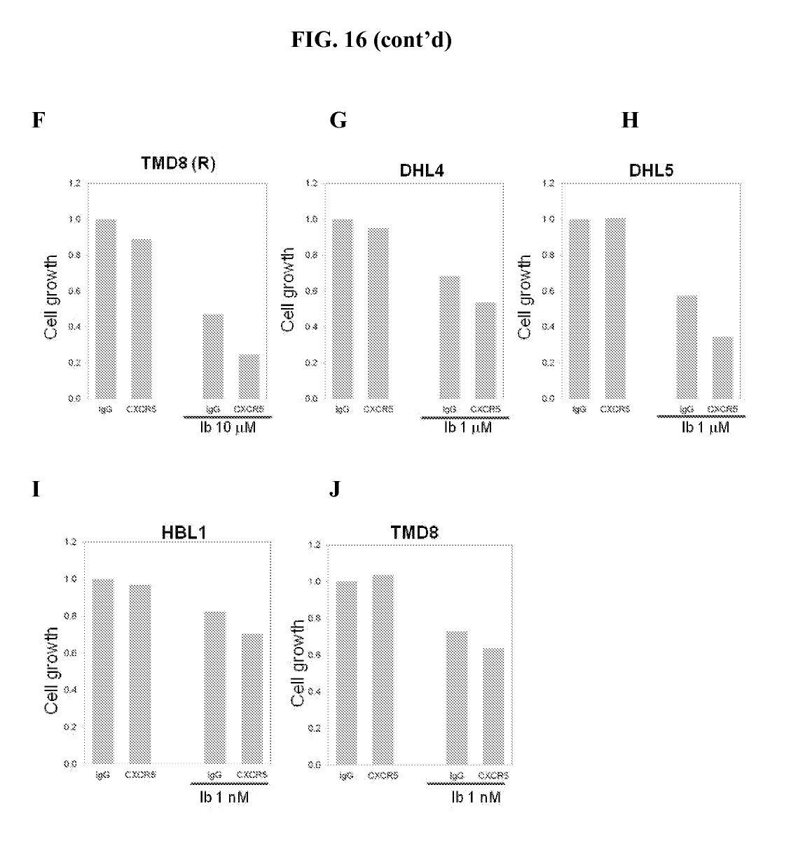

8. Ibrutinib with the PD-L1 or PD-L2 antibodies (FIG. 16).

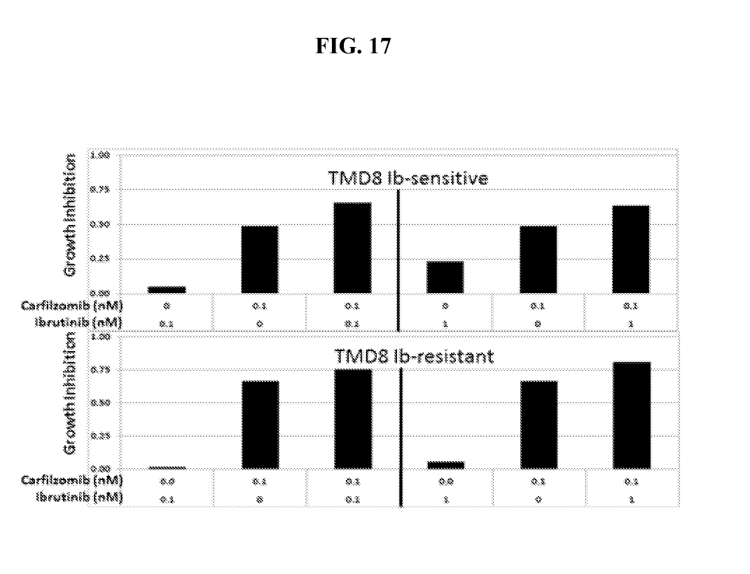

9. Ibrutinib with a CXCR5 antibody (FIG. 17).

### Example 2

### High Throughput Screen of BTK Inhibitor with 99 Anti-Cancer Agents

A high throughput screen of 17 Diffuse Large B Cell Lymphoma (DLBCL) cell lines was conducted for their response to Ibrutinib in combination with 99 anti-cancer agents selected from among standard-of-care and emerging therapeutics and targeted agents. The goal of the project was to identify and quantify specific synergies with Ibrutinib to identify pathways that contribute to clinical response. Examples of therapeutics tested included first-line DLBCL therapeutics: RCHOP (Rituximab, Cyclophosphamide, Doxorubicin, Vincristine, Prednisone) or EPOCH (+Etoposide) and Second-line therapeutics: Dexamethasone, Prednisone, Etoposide, Vincristine, Gemcitabine, Carboplatin, Ifosfamide, Bendamustine, Cyclophosphamide, Rituximab, Lenalidomide, and Anthracycline.

The 17 DLBCL cell lines tested were DB, DOHH-2, HBL-1, HT, NU-DHL-1, OCI-Ly1, OCI-Ly10, OCI-Ly18, OCI-Ly19, OCI-Ly3, OCI-Ly7, Pfeiffer, SU-DHL-5, SU-DHL-6, SU-DHL-8, TMD8 and Toledo. Eight of the cell lines were screened in human MSC-conditioned medium (hMSC-CM) and nine of the cell lines were screened with hMSC-CM+1 ug/ml each of anti-IgM and anti-IgG. The assay was performed in a 384-well format (6×6 HFDR format) with intra-plate replicates (Ibrutinib n=4; enhancers n=2; combination n=1), inter-plate replicates (n=3) and 20 self-crosses. The dose-response matrix screening was designed to detect both types of multi-target interaction, potency shifts or efficacy boosts.

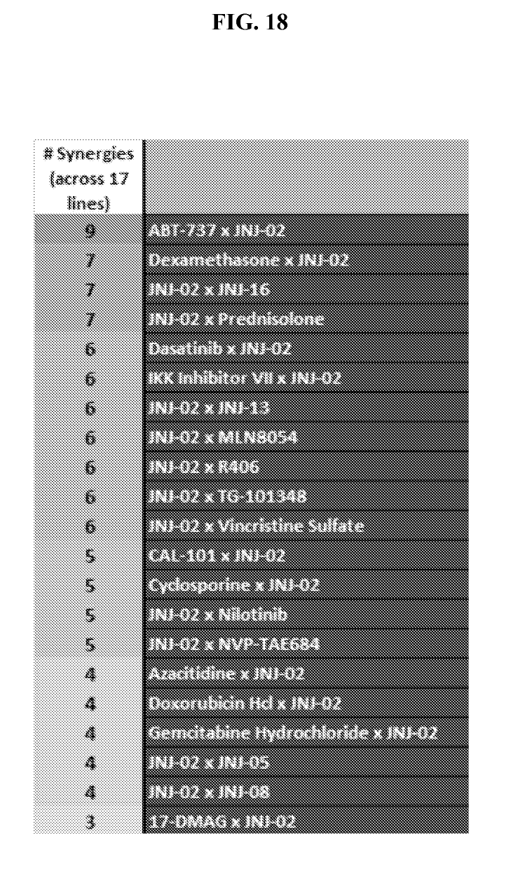

The cells were seeded 24 h before dosing. Cells were dosed with ibrutinib (JNJ-02) and the test compounds at varying concentrations as depicted in FIGS. 18-39. At T0 (0 h after dosing) and T72 (72 h after dosing) ATP-lite raw values were obtained. Growth inhibition of the cell culture was measured as follows:

Measure untreated at time 0 (V0) (the time at which drugs are added), treated (T) and untreated (V) (at assay end point (72 h).

If T>V0−100%*1−[(T−V0)/(V−V0)]

If T<V0−100%*1−[(T−V0)/V0]

0% (no growth inhibition)—treatment viability signal and 72 h vehicle viability signal are matched. (T=V)

100% (Total growth inhibition)—treatment viability signal and 0 h vehicle viability signal are matched. (T=V0)

200% (complete kill)—treatment viability signal is 0. (T=0)

The growth inhibition measure is sensitive to the cell doubling time (e.g., it measures the fraction of (net) growth inhibition during the assay period). Growth inhibition gives additional valuable information. For example, 0%-100% (growth inhibition) represents % reduction in net increase in the cells with vehicle during drug incubation period, 100% represents no net increase in viability signal at T72 and T0) (i.e., cytostatic) and 100%-200% (killing zone) represents cytotoxic effects.

Combination effects, including synergistic effects, with Ibrutinib were observed with both standard-of-care and emerging therapeutics.

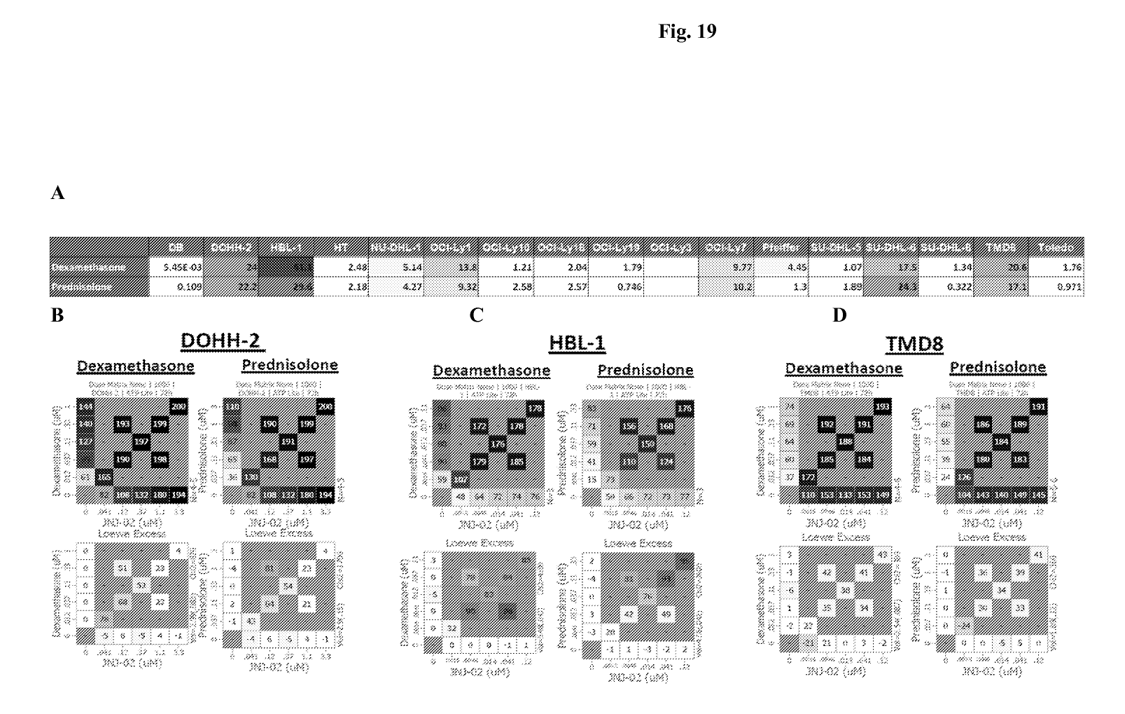

Combination effects of ibrutinib with glucocorticoids Dexamethasone and Prednisolone are shown in FIG. 19.

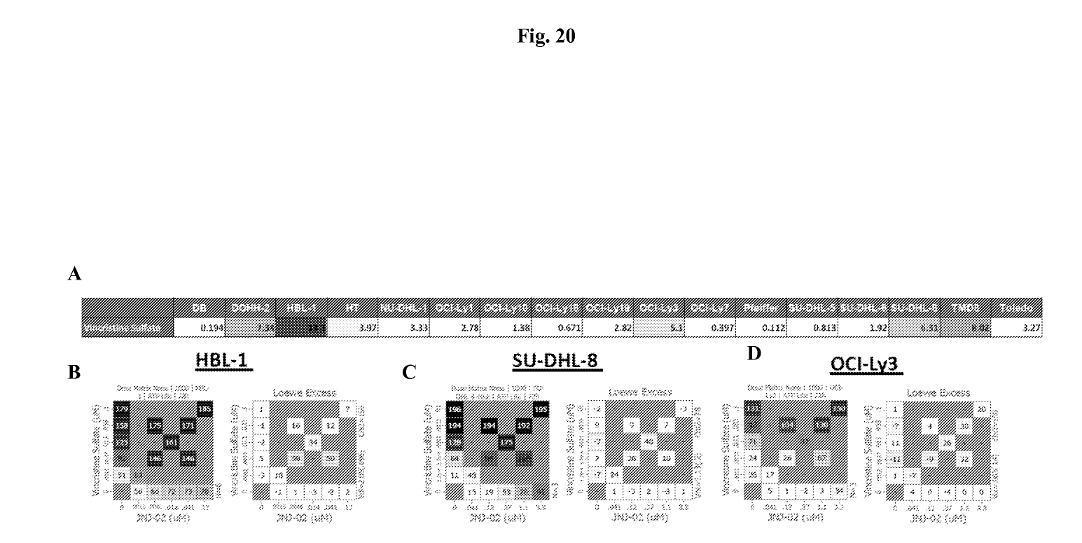

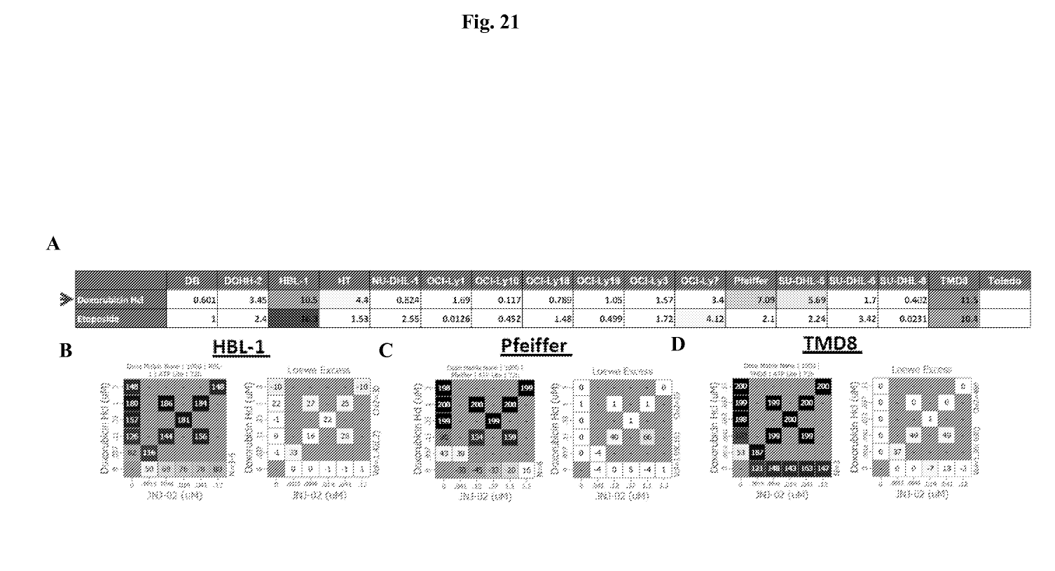

Combination effects of ibrutinib with the Vinca Alkaloid Vincristine and TOPO II Inhibitors, Doxorubicin and Etoposide, are shown in FIGS. 20 and 21.

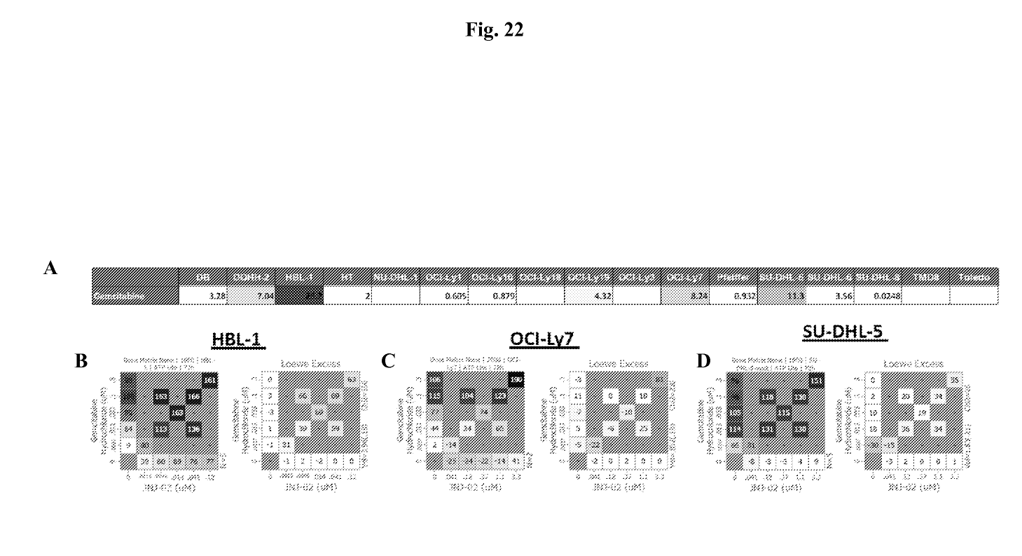

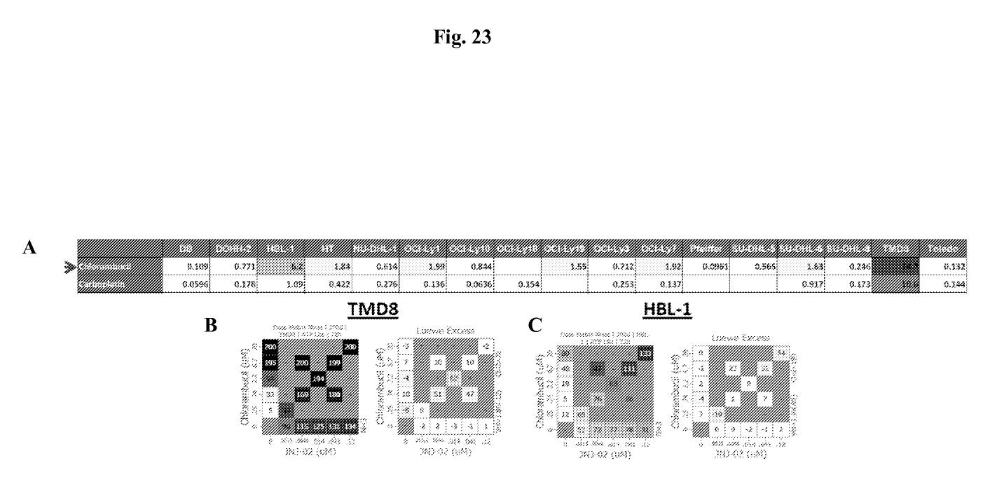

Combination effects of ibrutinib with Anti-metabolite Gemcitabine and DNA Alkylating/Damaging Agents, Carboplatin and Chlorambucil, are shown in FIGS. 22 and 23.

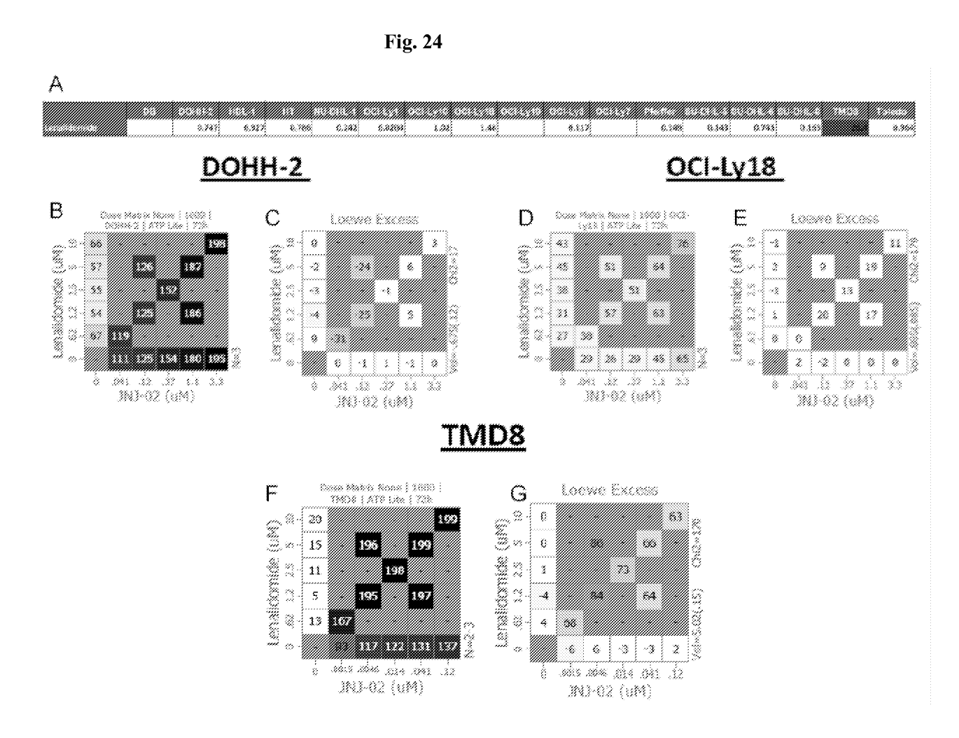

Combination effects of ibrutinib with Lenalidomide are shown in FIG. 24. Lenalidomide was not active as a single agent but synergized with ibrutinib.

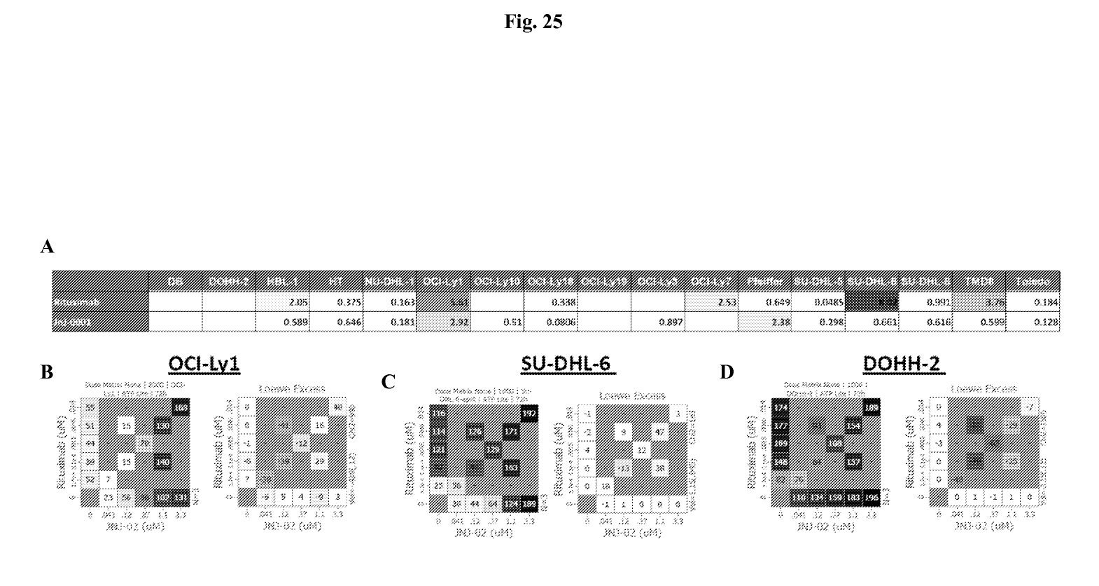

Combination effects of ibrutinib with the anti-CD20 antibody Rituximab are shown in FIG. 25.

Combination effects of ibrutinib with the SYK inhibitor R406 are shown in FIG. 26.

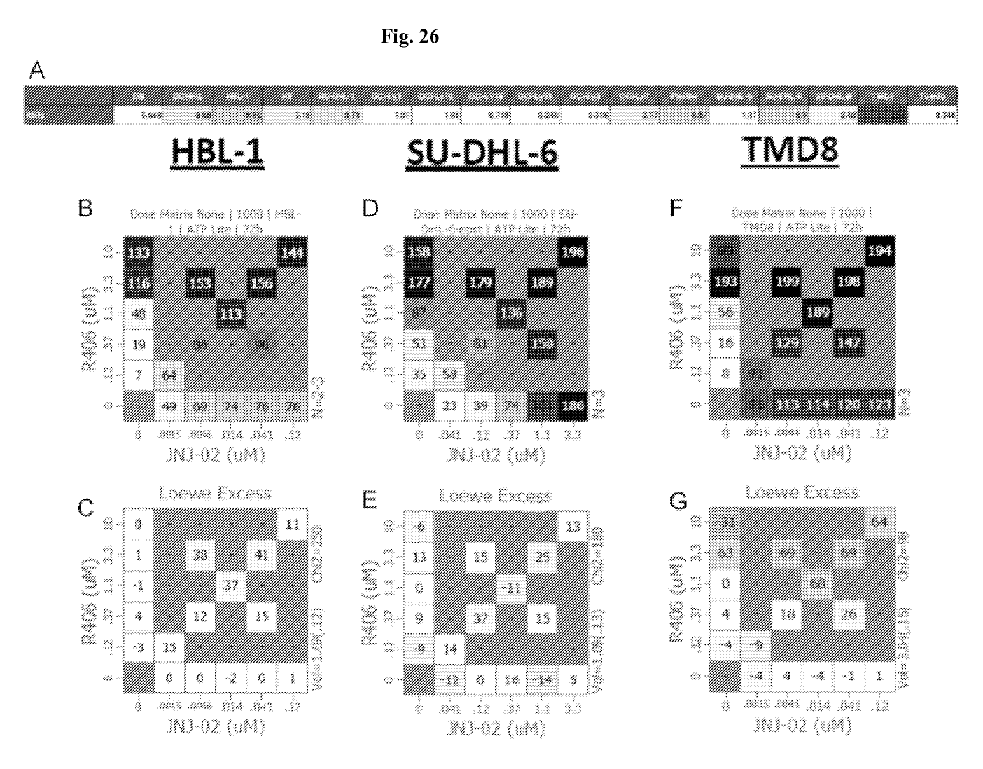

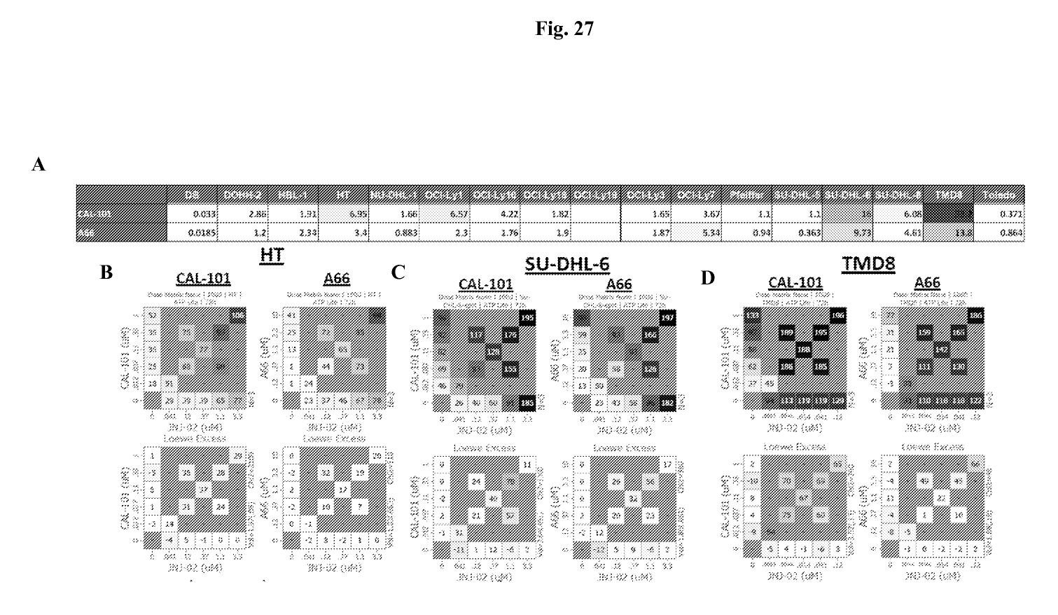

Combination effects of ibrutinib with PI3K pathway inhibitors CAL-101 and A66 R406 are shown in FIG. 27.

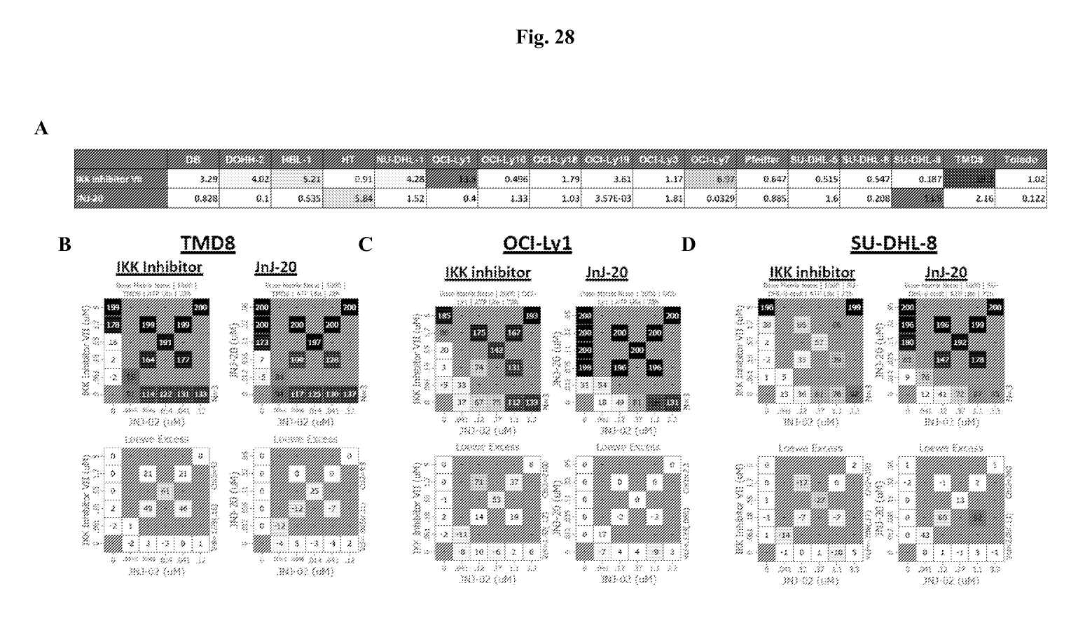

Combination effects of ibrutinib with NF-κB Pathway Inhibitors, IKK Inhibitor VII and JNJ-20, are shown in FIG. 28.

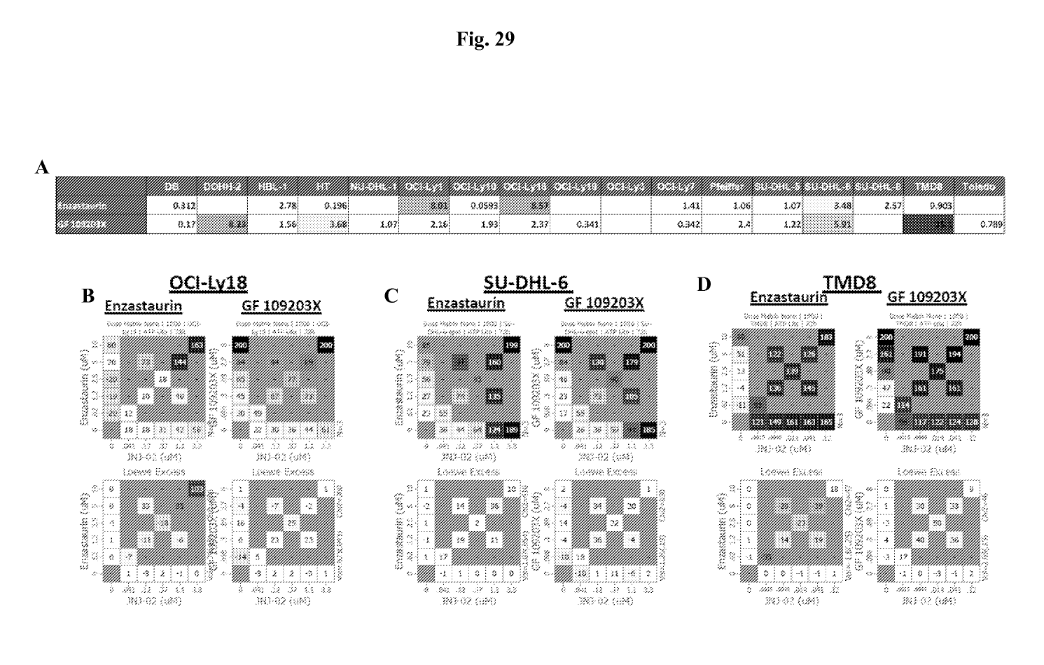

Combination effects of ibrutinib with PKC Perturbagens, Enzastarin and GF109203X, are shown in FIG. 29.

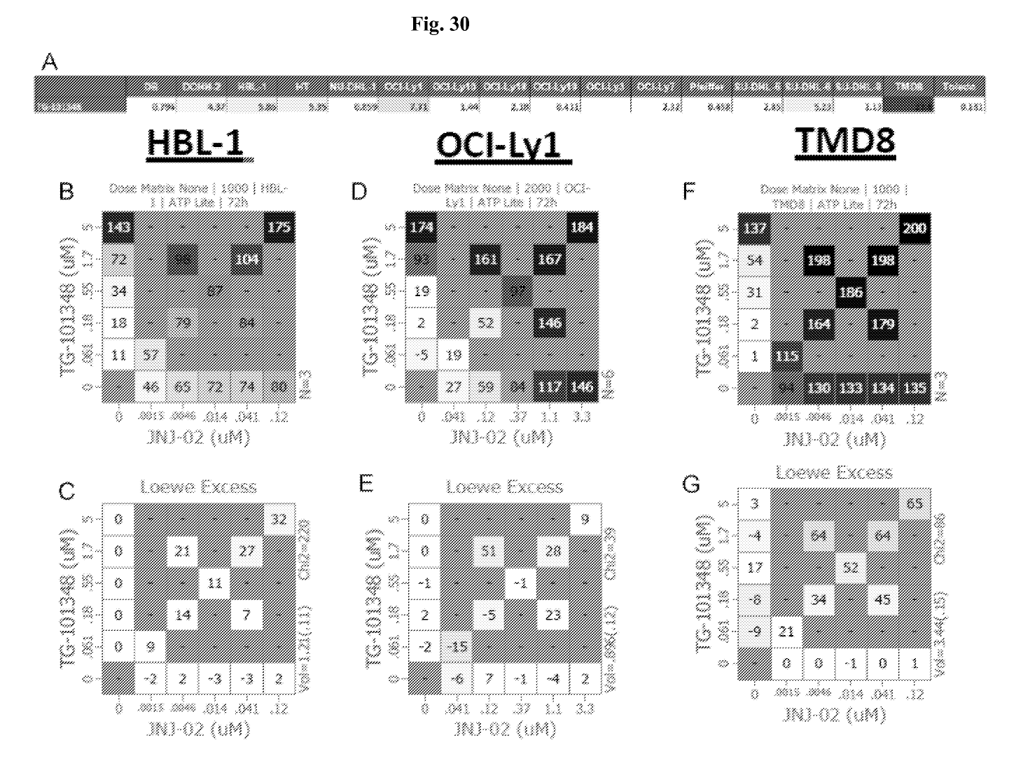

Combination effects of ibrutinib with the JAK Inhibitor TG-101348 are shown in FIG. 30.

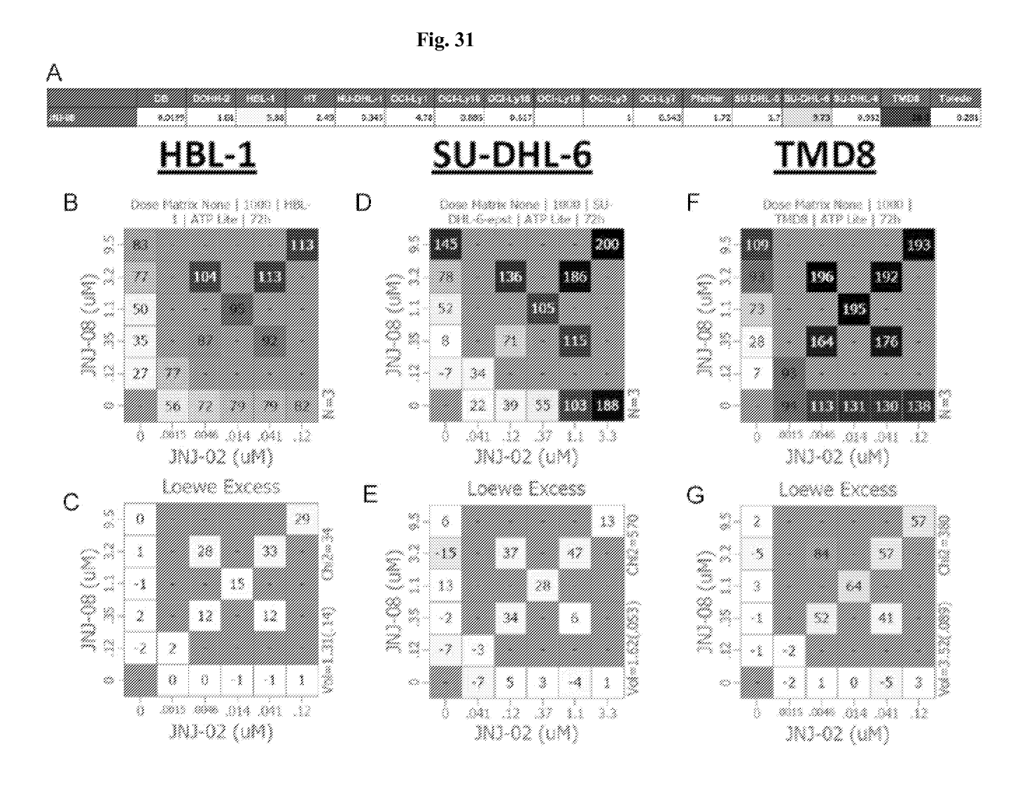

Combination effects of ibrutinib with Cdk4/6 inhibitor JNJ-08 are shown in FIG. 31.

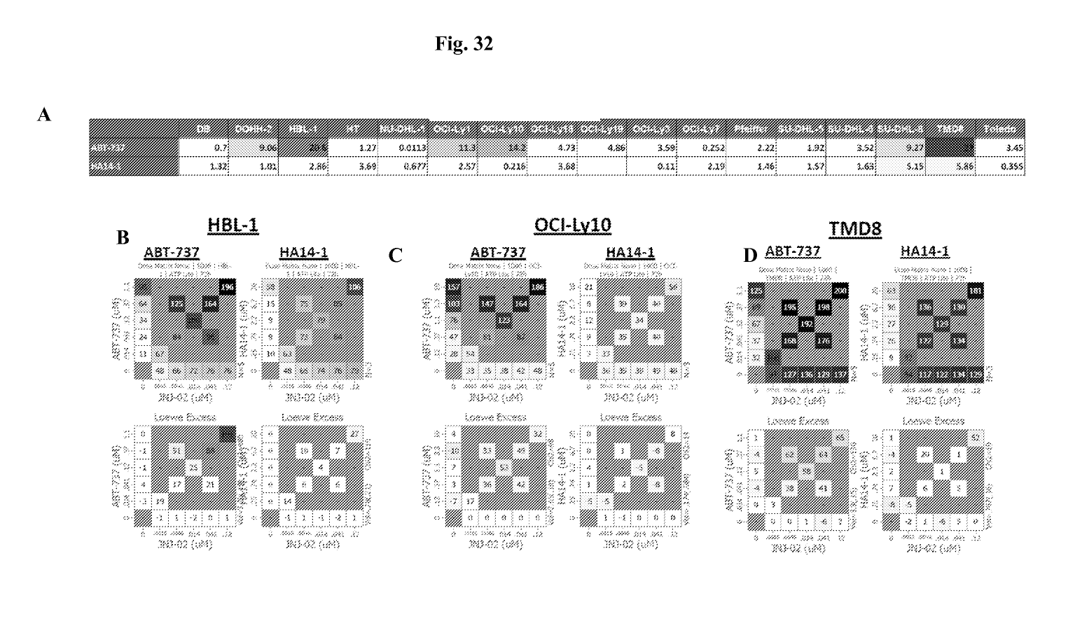

Combination effects of ibrutinib with BCL2 Inhibitors, ABT-737 and HA14-1, are shown in FIG. 32.

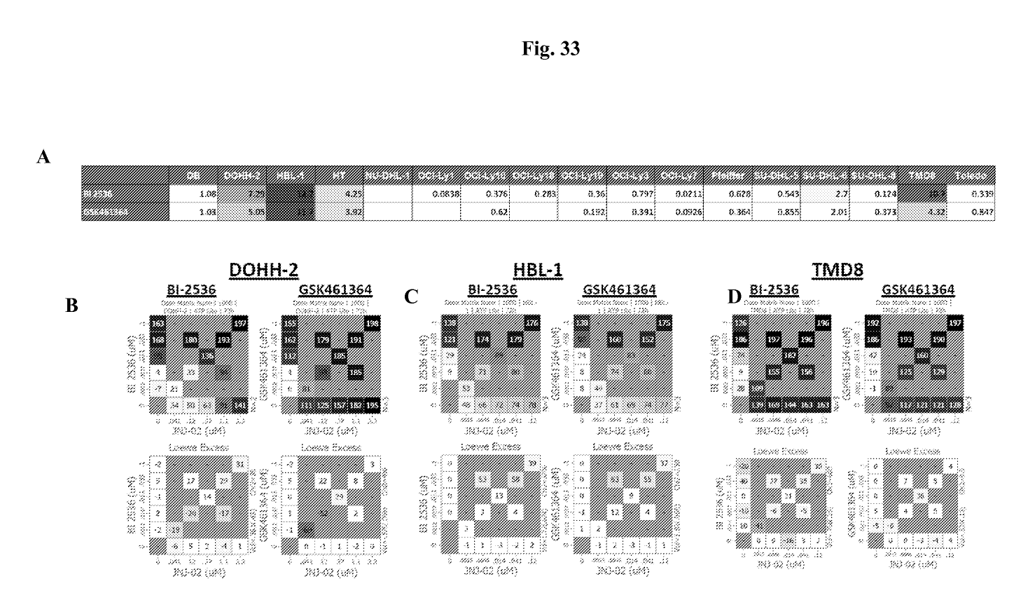

Combination effects of ibrutinib with PLK1 Inhibitors, BI-2536 and GSK461364, are shown in FIG. 33.

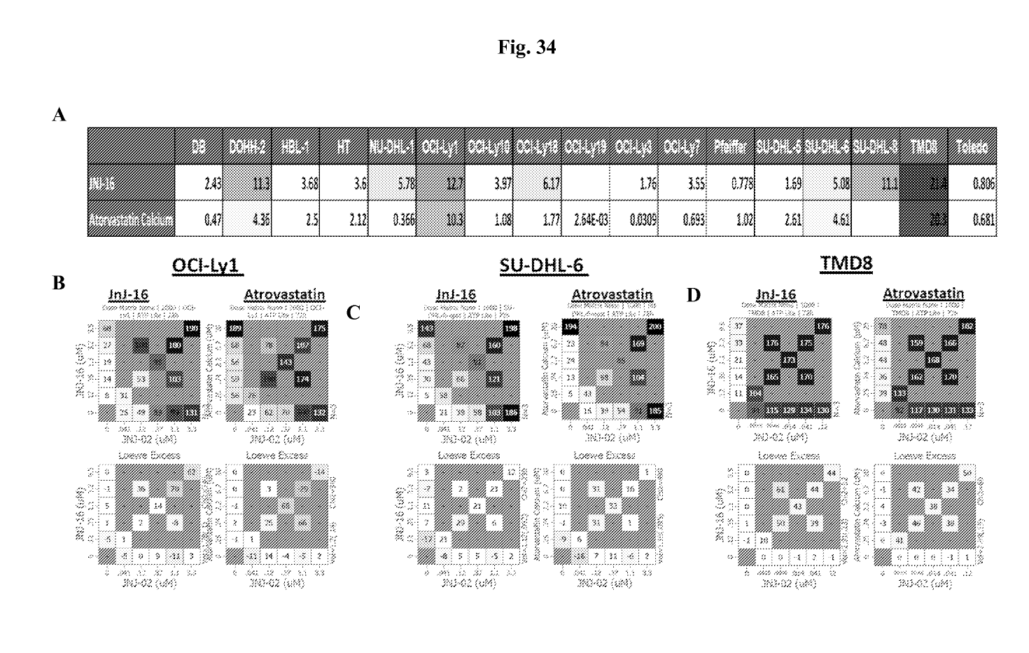

Combination effects of ibrutinib with the GLS inhibitors JNJ-16 and Atrovastatin are shown in FIG. 34.

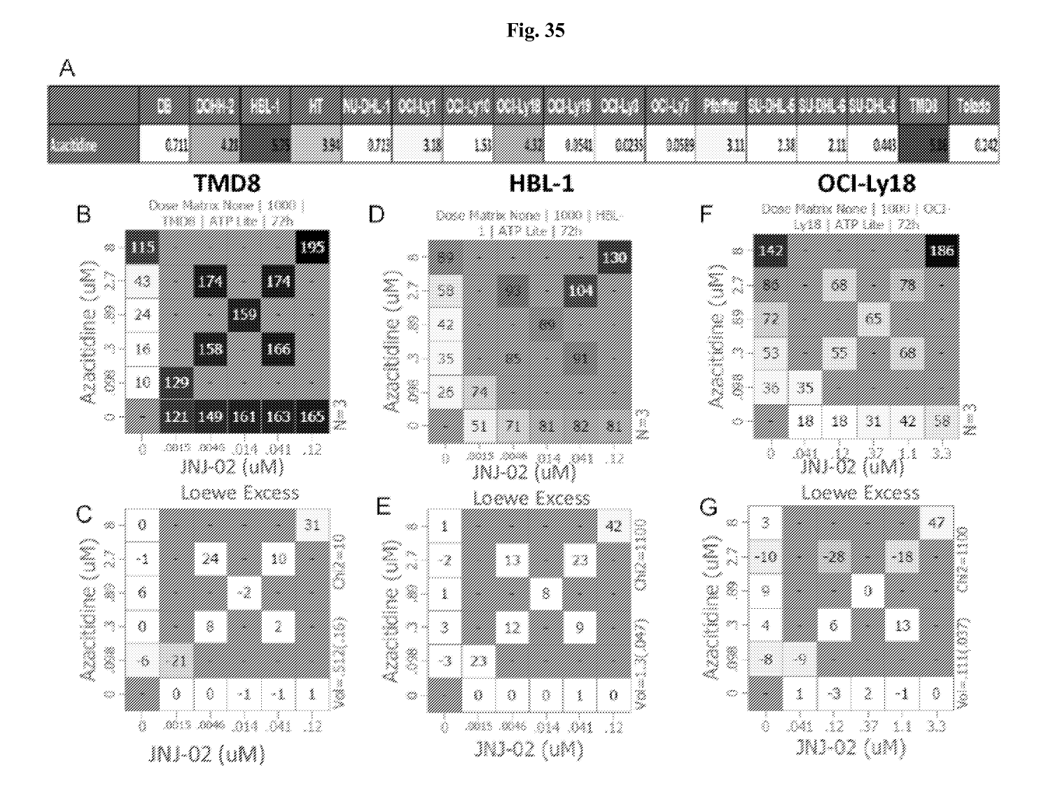

Combination effects of ibrutinib with the DNA Methyltransferase inhibitor Azacitidine are shown in FIG. 35.

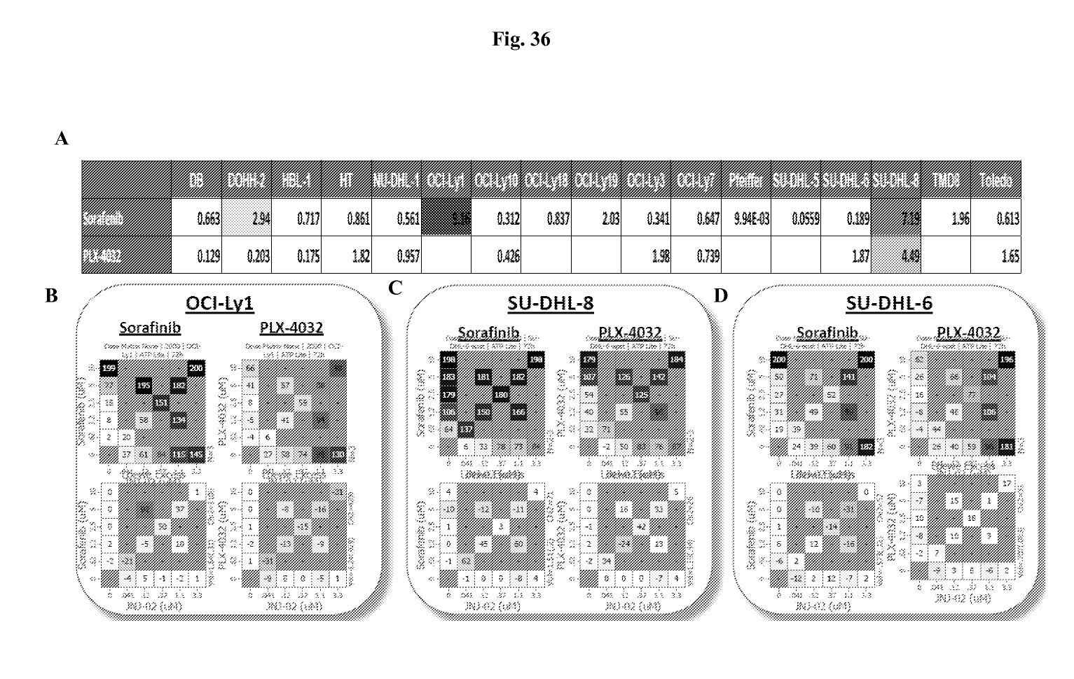

Combination effects of ibrutinib with the Ras/MAPK Pathway Inhibitors, Sorafinib and PLX-4032, are shown in FIG. 36.

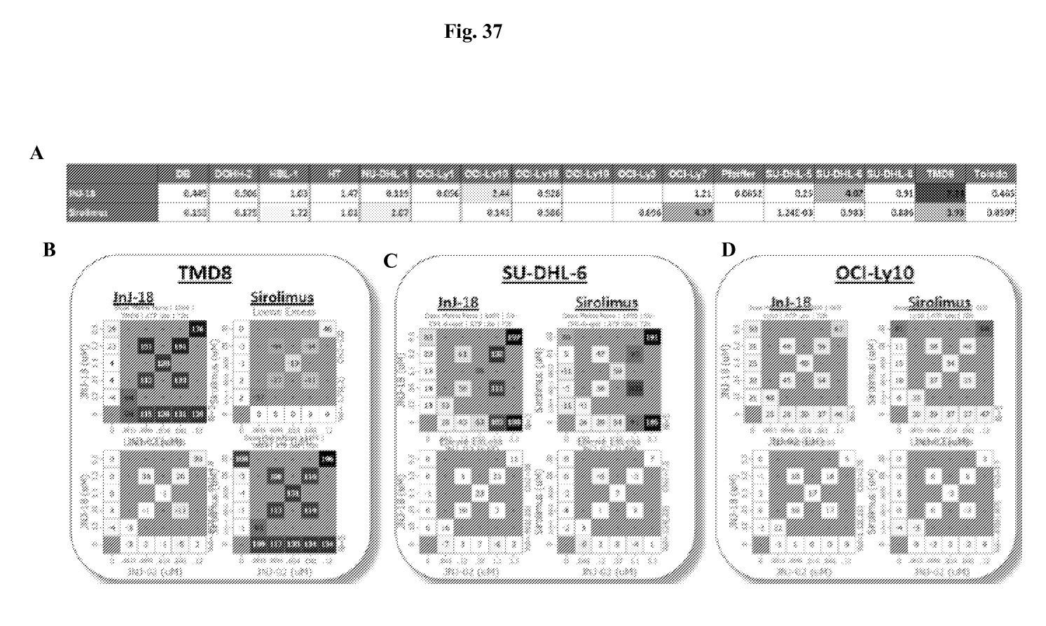

Combination effects of ibrutinib with the AKT/mTOR Pathway Inhibitors, JNJ-18 and Sirolimus, are shown in FIG. 37.

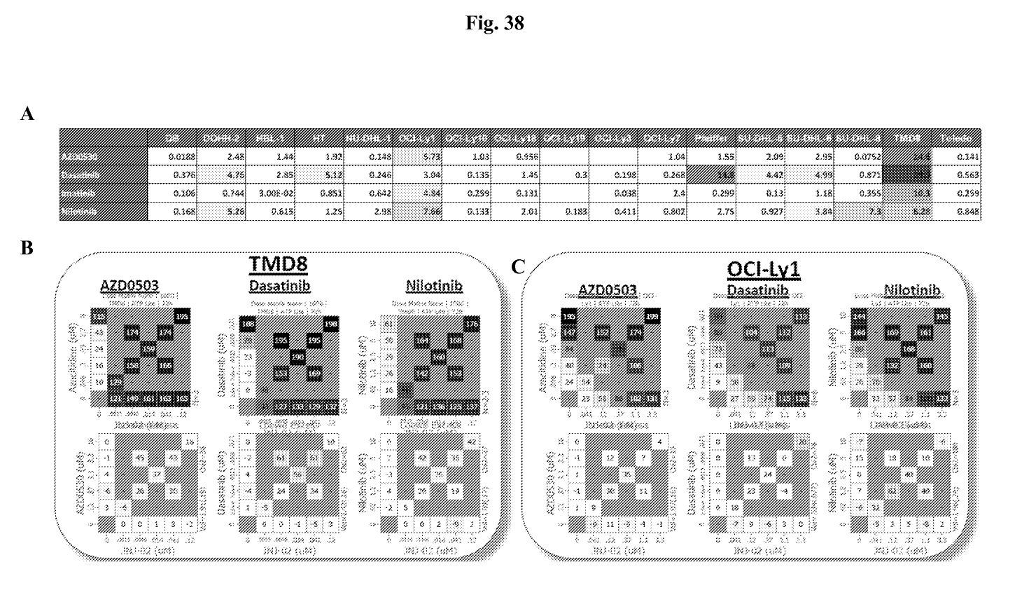

Combination effects of ibrutinib with Tyrosine Kinase Receptor Inhibitors, AZD0530, Dasatinib, Imatinib, and Nilotinib are shown in FIG. 38.

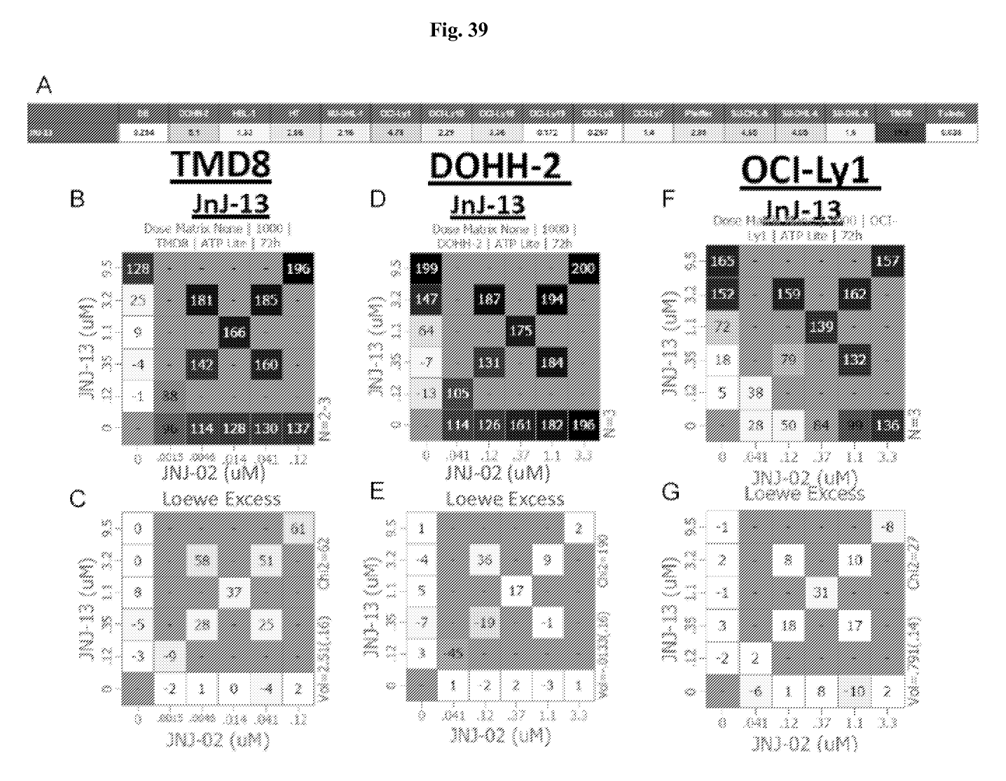

Combination effects of ibrutinib with the FGFR1 tyrosine kinase inhibitor JNJ-13 are shown in FIG. 39.

The examples and embodiments described herein are illustrative and various modifications or changes suggested to persons skilled in the art are to be included within this disclosure. As will be appreciated by those skilled in the art, the specific components listed in the above examples may be replaced with other functionally equivalent components, e.g., diluents, binders, lubricants, fillers, and the like.

What is claimed is:

1. A method for treating a B-cell proliferative disorder comprising administering to a subject in need thereof a therapeutically effective amount of a combination comprising:
a. Ibrutinib; and
b. an anticancer agent, wherein the anticancer agent inhibits Bcl-2; Janus kinase 2 (JAK2); Anaplastic lymphoma kinase (ALK); or heat shock protein 90 (Hsp90),
wherein the combination provides a synergistic therapeutic effect compared to administration of ibrutinib or the anticancer agent alone.

2. The method of claim 2, wherein the anticancer agent inhibits Bcl-2.

3. The method of claim 3, wherein the anticancer agent that inhibits Bcl-2 is selected from ABT-737, ABT-199 and HA14-1.

4. The method of claim 1, wherein the anticancer agent inhibits JAK2.

5. The method of claim 4, wherein the anticancer agent that inhibits JAK2 is TG-101348.

6. The method of claim 1, wherein the anticancer agent inhibits ALK.

7. The method of claim 6, wherein the anticancer agent that inhibits ALK is NVP-TAE684.

8. The method of claim 1, wherein the anticancer agent inhibits Hsp90.

9. The method of claim 8, wherein the anticancer agent that inhibits Hsp 90 is 17-DMAG.

10. A method for treating a B-cell proliferative disorder comprising administering to a subject in need thereof a therapeutically effective amount of a combination comprising:
a. Ibrutinib; and
b. an anticancer agent, wherein the anticancer agent is a glucocorticoid, a vinca alkaloid, an anti-metabolite, a DNA damaging agent, lenalidomide, rituximab, or a PKC perturbagen,
wherein the combination provides a synergistic therapeutic effect compared to administration of ibrutinib or the anticancer agent alone.

11. The method of claim 10, wherein the anticancer agent is a glucocorticoid.

12. The method of claim 10, wherein the anticancer agent is a vinca alkaloid.

13. The method of claim 10, wherein the anticancer agent is an anti-metabolite.

14. The method of claim 10, wherein the anticancer agent is a DNA damaging agent.

15. The method of claim 10, wherein the anticancer agent is a PKC perturbagen.

16. The method of claim 15, wherein the PKC perturbagen is selected from enzastarin and GF109203X.

17. A method for treating a B-cell proliferative disorder comprising administering to a subject in need thereof a therapeutically effective amount of a combination comprising:
a. Ibrutinib; and
b. an anticancer agent, wherein the anticancer agent inhibits a B-cell receptor pathway kinase selected from among Lyn/Fyn, Syk, PI3K, PKCβ, and IKK,
wherein the combination provides a synergistic therapeutic effect compared to administration of ibrutinib or the anticancer agent alone.

18. The method of claim 17, wherein the anticancer agent inhibits a B-cell receptor pathway kinase selected from among Lyn/Fyn, Syk, PI3K, PKCβ, and IKK.

19. The method of claim 18, wherein the anticancer agent inhibits Lyn/Fyn.

20. The method of claim 18, wherein the anticancer agent inhibits Syk.

21. The method of claim 18, wherein the anticancer agent inhibits PKCβ.

22. The method of claim 18, wherein the anticancer agent inhibits IKK.

23. The method of claim 18, wherein the anticancer agent inhibits PI3K.

24. The method of claim 23, wherein the anticancer agent that inhibits PI3K is selected from IPI-145, BKM120, BEZ235, GDC-0941, AMG319, CAL-101 and A66.

25. A method for treating a B-cell proliferative disorder comprising administering to a subject in need thereof a therapeutically effective amount of a combination comprising:
a. a therapeutically effective amount of Ibrutinib; and
b. an anticancer agent, wherein the anticancer agent inhibits the 20s proteasome, IRF-4, IRAK4, EZH2, CXCR4, CXCR5, GLS, cyclin dependent kinase 4/6 (CDK4/6), topoisomerase II, PLK; DNA methyltransferase, the Ras/MAPK pathway, or FGFR1 tyrosine kinase,
wherein the combination provides a synergistic therapeutic effect compared to administration of ibrutinib or the anticancer agent alone.

26. The method of any of claim 1, 10 or 25, wherein the B-cell proliferative disorder is diffuse large B-cell lymphoma (DLBCL), chronic lymphocytic leukemia (CLL), small lymphocytic lymphoma (SLL), high risk CLL, or a non-CLL/SLL lymphoma, follicular lymphoma, mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma, acute or chronic myelogenous (or myeloid) leukemia, myelodysplastic syndrome, or acute lymphoblastic leukemia.

27. The method of claim 26, wherein the B-cell proliferative disorder is DLBCL.

28. The method of claim 27, wherein the DLBCL is “activated B-cell” (ABC) DLBCL.

29. The method of claim 27, wherein the DLBCL is “germinal center B-cell like” (GCB) DLBCL.

30. The method of any of claim 1, 10 or 25, wherein Ibrutinib is administered in a therapeutically-effective amount.

31. The method of claim 30, wherein the therapeutically-effective amount of Ibrutinib is between about 10 mg to about 100 mg, 100 mg and about 200 mg, or about 200 to about 300 mg, or about 300 to about 500 mg, or about 500 to about 840 mg.

32. The method of claim 31, wherein the therapeutically-effective amount of Ibrutinib is about 140 mg.

33. The method of any of claim 1, 10 or 25, wherein Ibrutinib and the anticancer agent are in a combined dosage form.

34. The method of any of claim 1, 10 or 25, wherein Ibrutinib and the anticancer agent are in separate dosage forms.

35. The method of any of claim 1, 10 or 25, wherein Ibrutinib and the anticancer agent are administered simultaneously, essentially simultaneously or within the same treatment protocol.

36. The method of any of claim 1, 10 or 25, wherein Ibrutinib and the anticancer agent are administered sequentially.

37. The method of any of claim 1, 10 or 25, wherein the anticancer agent is administered in an amount between about 5 mg to about 1000 mg.

38. The method of any of claim 1, 10 or 25, wherein the ratio of Ibrutinib to the anticancer agent is about 9:1, about 4:1, about 7:3, about 3:2, about 1:1, about 2:3, about 3:7, about 1:4, or about 1:9.

39. A pharmaceutical composition comprising:
a. a therapeutically effective amount of Ibrutinib; and
b. an anticancer agent, wherein the anticancer agent inhibits Bcl-2, Janus kinase 2 (JAK2), Anaplastic lymphoma kinase (ALK), or heat shock protein 90 (Hsp90); or the anticancer agent is a glucocorticoid, a vinca alkaloid, an anti-metabolite, a DNA damaging agent, lenalidomide, rituximab, or a PKC perturbagen; or the anticancer agent inhibits a B-cell receptor pathway kinase selected from among Lyn/Fyn, Syk, PI3K, PKCβ, and IKK; or the anticancer agent inhibits the 20s proteasome, IRF-4, IRAK4, EZH2, CXCR4, CXCR5, GLS, cyclin dependent kinase 4/6 (CDK4/6), topoisomerase II, PLK; DNA methyltransferase, the Ras/MAPK pathway, or FGFR1 tyrosine kinase; or the anticancer agent is selected from AZD0503, dasatinib and nilotinib, and JNJ-20;
wherein the combination provides a synergistic therapeutic effect compared to administration of ibrutinib or the anticancer agent alone.

40. The pharmaceutical composition of claim 39, wherein the composition further comprises a pharmaceutically acceptable carrier or an adjuvant.
